SEQUENCE LISTING (34 sequences)

SEQ ID NO: 1 - protein, 11 aa, Artificial
  Consensus motif
LGVNYLHLMPL

SEQ ID NO: 2 - protein, 6 aa, Artificial
  Consensus motif (2)
DGGYAV

SEQ ID NO: 3 - protein, 6 aa, Artificial
  Consensus motif
DFVFNH

SEQ ID NO: 4 - protein, 13 aa, Artificial
  Consensus motif
LREIFPDTAPGNF

SEQ ID NO: 5 - protein, 9 aa, Artificial
  Consensus motif
FNSYQWDLN

SEQ ID NO: 6 - protein, 12 aa, Artificial
  Consensus motif (6)
ILRLDAVAFLWK

SEQ ID NO: 7 - protein, 4 aa, Artificial
  Consensus motif
EAIV

SEQ ID NO: 8 - protein, 8 aa, Artificial
  Consensus motif
YVRCHDDI

SEQ ID NO: 9 - protein, 11 aa, Artificial
  Consensus motif (9)
RISGTLASLAG

SEQ ID NO: 10 - protein, 10 aa, Artificial
  Consensus motif (9)
GIPLIYLGDE

SEQ ID NO: 11 - protein, 6 aa, Artificial
  Consensus motif
RWVHRP

SEQ ID NO: 12 - protein, 96 aa, Artificial
  Consensus sequence
LGVNYLHLMPLDGGYAVDFVFNHLREIFPDTAPGNFFNSYQWDLNILRLDAVAFLWKEAIVYVRCHDDIRISGTLASLAGGIPLIYLGDERWVHRP

SEQ ID NO: 13 - protein, 628 aa, Neisseria polysaccharea
SPNSQYLKTRILDIYTPEQRAGIEKSEDWRQFSRRMDTHFPKLMNELDSVYGNNEALLPMLEMLLAQAWQSYSQRNSSLKDIDIARENNPDWILSNKQVGGVCYVDLFAGDLKGLKDKIPYFQELGLTYLHLMPLFKCPEGKSDGGYAVSSYRDVNPALGTIGDLREVIAALHEAGISAVVDFIFNHTSNEHEWAQRCAAGDPLFDNFYYIFPDRRMPDQYDRTLREIFPDQHPGGFSQLEDGRWVWTTFNSFQWDLNYSNPWVFRAMAGEMLFLANLGVDILRMDAVAFIWKQMGTSCENLPQAHALIRAFNAVMRIAAPAVFFKSEAIVHPDQVVQYIGQDECQIGYNPLQMALLWNTLATREVNLLHQALTYRHNLPEHTAWVNYVRSHDDIGWTFADEDAAYLGISGYDHRQFLNRFFVNRFDGSFARGVPFQYNPSTGDCRVSGTAAALVGLAQDDPHAVDRIKLLYSIALSTGGLPLIYLGDEVGTLNDDDWSQDSNKSDDSRWAHRPRYNEALYAQRNDPSTAAGQIYQDLRHMIAVRQSNPRFDGGRLVTFNTNNKHIIGYIRNNALLAFGNFSEYPQTVTAHTLQAMPFKAHDLIGGKTVSLNQDLTLQPYQVMWLEIA

SEQ ID NO: 14 - DNA, 29 bp, Artificial
  primer
accctgcgcgaannnttccccgaccagca

SEQ ID NO: 15 - DNA, 29 bp, Artificial
  primer
tgctggtcggggaannnttcgcgcagggt

SEQ ID NO: 16 - DNA, 33 bp, Artificial
  primer
tatggatgcggttnnntttatttggaaacaaat

SEQ ID NO: 17 - DNA, 33 bp, Artificial
  primer
atttgtttccaaataaannnaaccgcatccata

SEQ ID NO: 18 - DNA, 33 bp, Artificial
  primer
tatggatgcggttgccnnnatttggaaacaaat

SEQ ID NO: 19 - DNA, 33 bp, Artificial
  primer
atttgtttccaaatnnnggcaaccgcatccata

SEQ ID NO: 20 - DNA, 34 bp, Artificial
  primer
tcaaatccgaagccnnngtccaccccgaccaagt

SEQ ID NO: 21 - DNA, 34 bp, Artificial
  primer
acttggtcggggtggacgatnnnttcggatttga

SEQ ID NO: 22 - DNA, 34 bp, Artificial
  primer
tcaaatccgaagccatcnnncaccccgaccaagt

SEQ ID NO: 23 - DNA, 34 bp, Artificial
  primer
acttggtcggggtgnnngatggcttcggatttga

SEQ ID NO: 24 - DNA, 32 bp, Artificial
  primer
tccgcagccacgacnnnatcggctggacgttt

SEQ ID NO: 25 - DNA, 32 bp, Artificial
  primer
aaacgtccagccgatnnngtcgtggctgcgga

SEQ ID NO: 26 - DNA, 27 bp, Artificial
  primer
acaggcgactgcnnngtcagtggtaca

SEQ ID NO: 27 - DNA, 27 bp, Artificial
  primer
tgtaccactgacnnngcagtcgcctgt

SEQ ID NO: 28 - DNA, 19 bp, Artificial
  primer
ccaacgaacacgaatgggc

SEQ ID NO: 29 - DNA, 34 bp, Artificial
  primer
ctgcgcgaannsnnscccgaccagcacccgggcg

SEQ ID NO: 30 - DNA, 34 bp, Artificial
  primer
ctggtcgggwnnwnnttcgcgcagggtgcggtcg

SEQ ID NO: 31 - DNA, 40 bp, Artificial
  primer
gcggttnnsnnwatttggaaacaaatggggacaagctgcg

SEQ ID NO: 32 - DNA, 42 bp, Artificial
  primer
ccaaatsnnwnnaaccgcatccatacgcaggatgtcaacgcc

SEQ ID NO: 33 - DNA, 34 bp, Artificial
  primer
tccgaagccnnsnnscaccccgaccaagtcgtcc

SEQ ID NO: 34 - DNA, 36 bp, Artificial
  primer
ggggtgwnnwnnggcttcggatttgaagaacacggc

(19) United States
(12) Patent Application Publication
(10) Pub. No.: US 2011/0086767 A1
(43) Pub. Date: Apr. 14, 2011

(54) MUTANTS OF GLYCOSIDE HYDROLASES AND USES THEREOF FOR SYNTHESIZING COMPLEX OLIGOSACCHARIDES AND DISACCHARIDE INTERMEDIATES

(75) Inventors: Laurence Mulard, Le Kremlin Bicetre (FR); Isabelle Andre, Toulouse (FR); Elise Champion, Cambridge, MA (US); Claire Moulis, Garidech (FR); Sandrine Morel, Auzeville (FR); Pierre Monsan, Mondonville (FR); Magali Remaud-Simeon, Ramonville (FR); Karine Descroix, Saint-Colomban (FR)

(73) Assignees: Institut Pasteur; Centre National De La Recherche Scientifique; Institut National Des Sciences Appliquees De Toulo use

(21) Appl. No.: 12/922,108

(22) PCT Filed: Mar. 12, 2009
(86) PCT No.: PCT/IB09/05325

§ 371 Date: Dec. 13, 2010

(30) Foreign Application Priority Data
Mar. 12, 2008 (EP) 08290237.0

(51) Int. Cl. C40B 30/02; C12P 19/12; C12N 9/24; C07H 21/04; C12N 15/63; C07H 15/04

(52) U.S. Cl. 506/8; 435/100; 435/200; 536/23.2; 435/320.1; 536/17.2

(57) ABSTRACT

Method for preparing the disaccharide α-D-glucopyranosyl-(1→4)-2-N-acetyl-2-deoxy-α-D-glucopyranoside, comprising the step of using a mutant of a wild type glycoside hydrolase.

The present invention relates to mutants of glycoside hydrolases and uses thereof in chemo-enzymatic synthesis of complex oligosaccharides, in particular fragments of S. flexneri 1a and 1b O-antigen.

Carbohydrates displayed at the surface of cells and pathogens are involved in a wide range of biological processes, among which several intercellular recognition events, as well as host-pathogen interactions possibly resulting in microbial or viral infections. The understanding of the molecular events involved in carbohydrate-mediated interactions has long been impaired by the difficult access to relevant oligosaccharides and glycoconjugates in pure form and sufficient amounts. The exquisite diversity of possible structures, varying in monosaccharide composition, linkage and branching pattern (ref. 1), is indeed a major roadblock to easy availability. In recent years however, important developments in the preparation of carbohydrate derivatives, based on (i) multistep chemical synthesis, (ii) enzymatic strategies using recombinant glycosyltransferases (i.e., enzymes catalyzing the transfer of a monosaccharide residue from an activated sugar phosphate to an acceptor molecule; EC 2.4) with in situ regeneration of sugar nucleotides, (iii) combinations thereof, or (iv) biosynthesis using metabolically engineered cell-factories (ref. 2), have opened the way to significant progress in the fields of glycobiology and glycotherapeutics (ref. 3-5). A number of efficient and elegant synthetic methods such as one-pot oligosaccharide synthesis (ref. 6) or automated synthetic tools (ref 7) have been developed to provide more straightforward access to structurally-defined carbohydrates. The use of lightly protected precursors and the regioselective one-pot protection of monosaccharides were recently emphasized (ref 8).

Nevertheless, despite accomplished advancements, chemical approaches towards specific usable microbial oligosaccharides still need considerable effort. They remain, for the most part, highly dependent on the design of appropriate combinations of multiple protection, deprotection, and efficient glycosylation steps, which often involve numerous tedious chromatographic separations (ref 9). Avoiding the need for protecting groups, organisms engineered to express several glycosyltransferase genes have been used to produce a nice range of biologically active complex carbohydrates, but they remain to date limited to the synthesis of short oligosaccharides which can passively cross the cell membrane (ref. 2, 10).

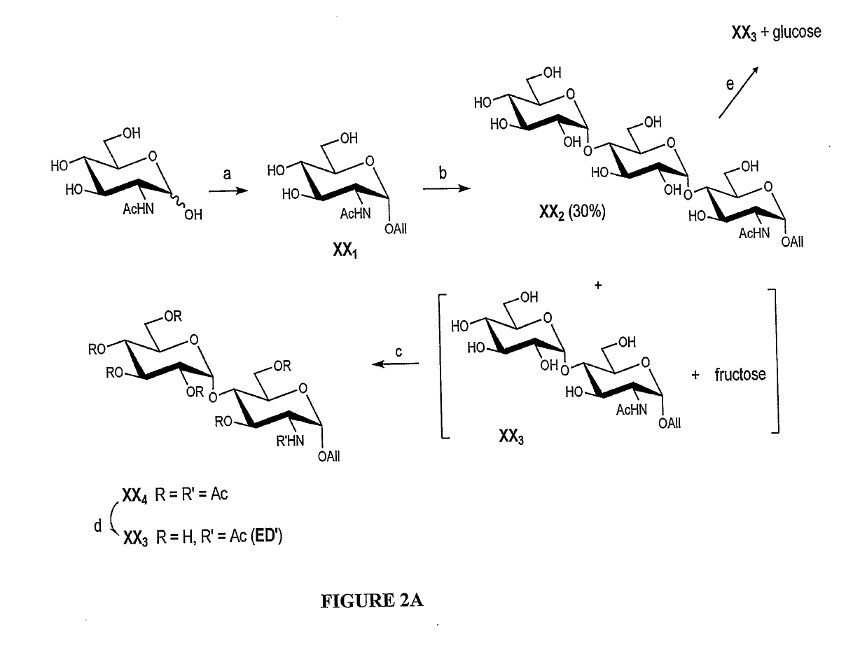

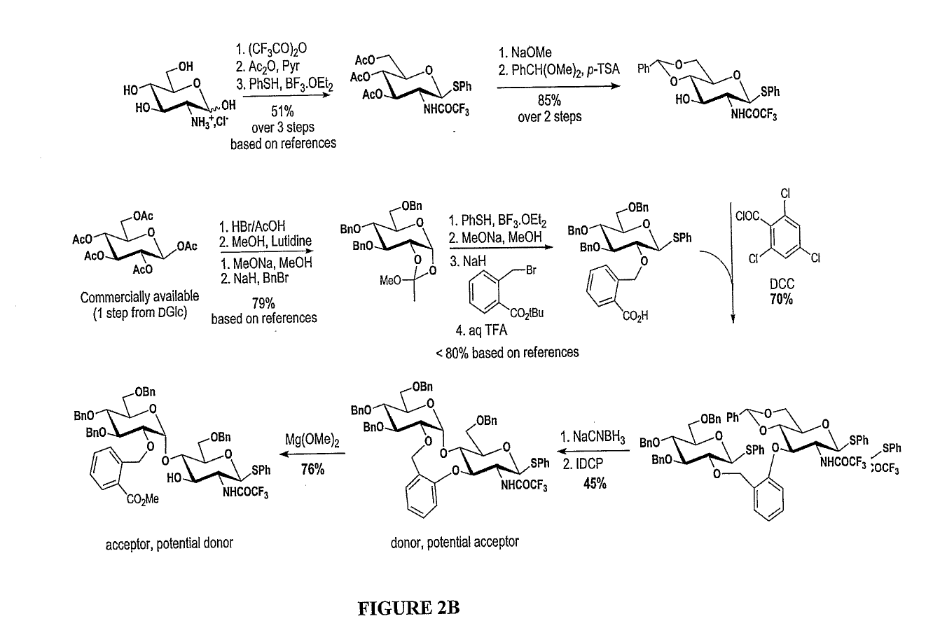

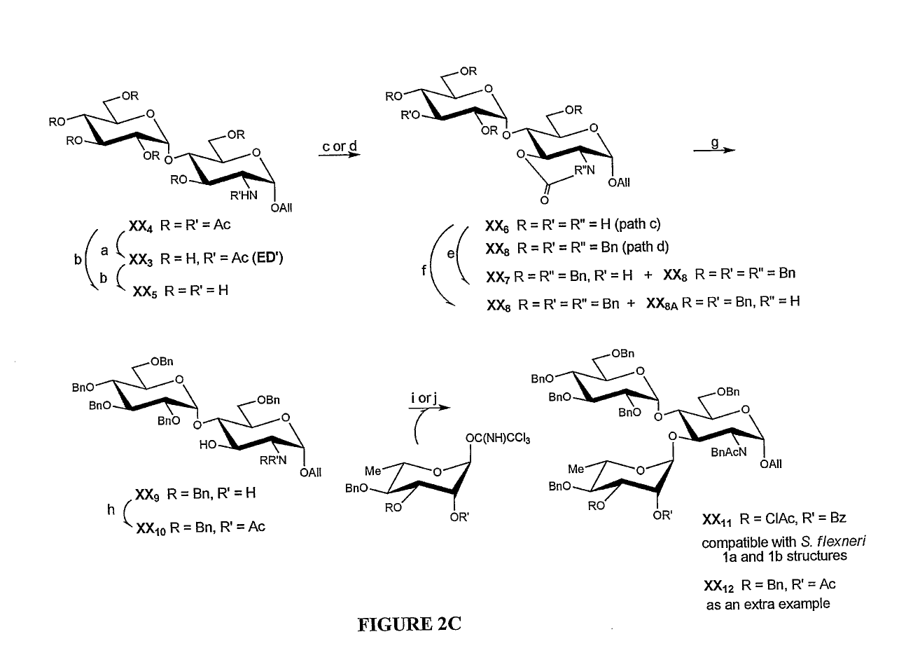

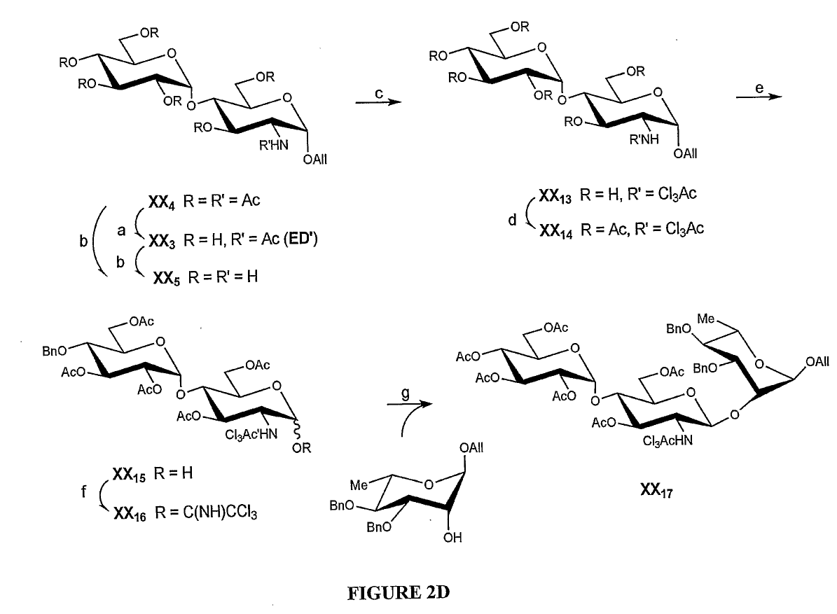

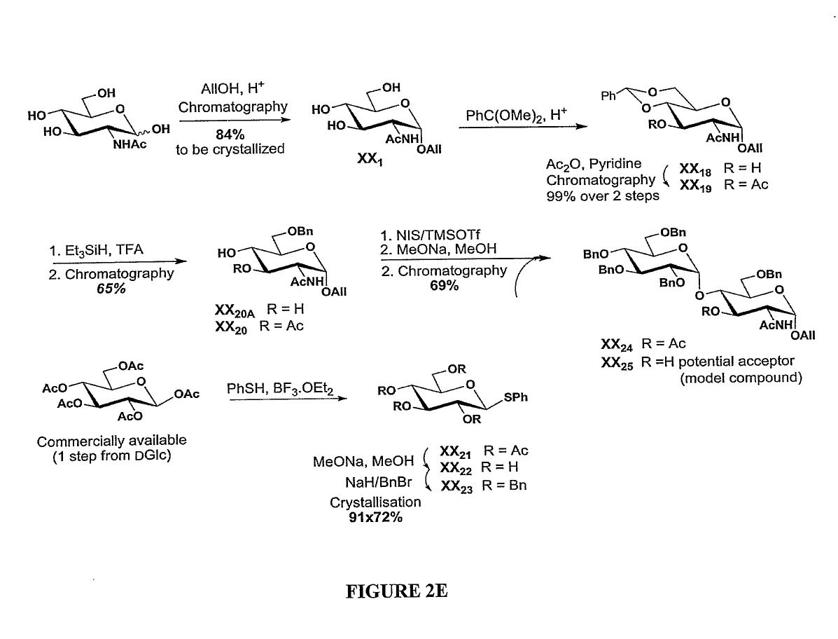

Following the early success of polysaccharide vaccines in the second half of the 20^{th} century, polysaccharide-protein conjugate vaccines were seen as a major progress in antibacterial vaccination (ref. 11, 12). Indeed, made from bacterial polysaccharides purified from pathogen cell cultures, eventually shortened following partial-chemical hydrolysis or enzymatic depolymerisation of the native antigen, and subsequently covalently coupled to a protein carrier, these second generation carbohydrate vaccines are suitable for use in human (ref. 12). Potential extrapolations are numerous since for a large number of pathogens, surface carbohydrates behave as key “protective antigens”. Interestingly, this long known property of a range of bacterial polysaccharides was extended in recent years to other microbial carbohydrates of fungal (ref. 13) and parasitic origin (ref. 14). Besides, the disclosure that cancer cells could among other features be differentiated from healthy ones by the presence of surface glycoconjugates, often termed tumor-associated carbohydrate antigens, contributed to additional interest in carbohydrate antigens (ref. 15). Overall, interest in synthetic carbohydrate-based vaccines has emerged as one amongst the many exploding fields of carbohydrate medical applications (ref. 15, 16). The development of synthetic microbial carbohydrate-protein conjugates, thus termed third generation carbohydrate vaccines, was proposed as an alternative to conventional polysaccharide vaccines, compatible with the increasingly demanding requirements in terms of safety, efficiency, product definition, and needs for multivalent vaccines (ref. 17). Most interestingly, use of the natural antigen, and consequently risks associated to materials of biological origin, are avoided. However, chemical synthesis of carbohydrate-protein conjugates, more precisely of appropriate carbohydrate haptens can be seen as a drawback. By way of example, FIG. 2B shows the first steps of a known chemical synthetic route to S. flexneri 1a O-antigen (ref. 66). In order to control the 1,2-cis glycosidic linkage involved in the key disaccharide motif α-D-glucopyranosyl-(1→4)-β-D-2-N-acetyl-2-deoxy-glucopyranosyl, the synthesis relies on the recently developed method of intramolecular glycosylation through prearranged unsymmetrically tethered glycosides. This attractive strategy involves a thioethyl glucopyranoside bearing an hydroxyl group at position 2 differentiated from the others in order to allow introduction of the tether, next linked at position 3 of the glucosamine acceptor. Subsequent deblocking at position 4 of the glucosamine residue provided the donor/acceptor bis(glycoside). Intramolecular glycosylation gave the (1→4)-α-D-glucosidic linkage only, providing a suitable disaccharide donor in 45% yield over two steps. Next, transesterification at position 3 of the glucosamine residue provided a disaccharide acceptor in 34% yield over three steps. As expected, the glycosylation step was both highly stereoselective and high yielding (72%, α-anomer only). A limitation of the strategy, however, is the 8 step-synthesis of the required tethered donor from glucose, added to the 5 steps synthesis of the acceptor. More recently, an alternative was proposed on a model disaccharide (K. Descroix & L. Mulard, unpublished). In this case (FIG. 2E), the construction of the α-D-glucopyranosyl-(1→2)-α-D-2-N-acetyl-2-deoxy-glucopyranosyl motif involves a more conventional glycosylation step between the phenyl tetra-O-benzyl-1-thio-β-D-glucopyranoside donor (XX₂₁) and allyl 2-acetamido-3-O-acetyl-6-O-benzyl-2-deoxy-α-D-glucopyranoside (XX₂₀) as acceptor. Interestingly, the disaccharide acceptor XX₂₅, bearing the required 1,2-cis stereochemistry was obtained in 69% yield over two steps indicating a good stereoselectivity of the glucosylation step despite the absence of any participating group at position 2 of the donor. Nevertheless, preparation of disaccharide XX₂₅ from the free monosaccharide precursors required a total of 10 synthetic steps combined to 3 purifications.

The use of enzymes as catalysts has then emerged as a practical alternative to a number of limitations encountered in chemical synthesis (ref. 18, 19). Leloir-type glycosyltransferases and transglycosidases constitute the two major classes of enzymes that can be used for the synthesis of glycosidic linkages. Both are enzymes transferring a glycosyl group from a donor to an acceptor. Glycosyltransferases require nucleotide sugar as donor substrate whereas transglycosidases usually employ mono- and/or oligosaccharides as donor substrates.

The term “donor” refers to a molecule that provides a glycosyl moiety which will be transferred to an acceptor molecule.

The term “acceptor” refers to a molecule that will receive the glycosyl moiety through the formation of a chemical bond, preferentially C—O-linkage.

However, despite the increasing number of available transglycosidases and glycosyltransferases (Leloir type), the lack of appropriate enzymatic tools with requisite substrate specificity has prevented extensive exploration of the chemo-enzymatic strategies when dealing with complex bacterial carbohydrate antigens. The use of multiple overexpressed native glycosyltransferases was shown to be highly rewarding for the synthesis of the upstream pentasaccharide terminus of the Neisseria meningitidis lipo-oligosaccharide (ref. 20), but examples of enzymatic synthesis of complex carbohydrate remain scarce (ref. 21). Indeed, certain membrane Leloir-type glycosyltransferases are not easily available. Their nucleotide activated sugar substrates are expensive. They may be generated in situ, but the process necessitates additional enzymes (ref. 21).

Replacement of glycosyltransferases by transglycosidases has been proposed to proceed from different types of glycosyl donors, and to be compatible with a larger variety of acceptors (ref. 22). Interestingly, modified donors were occasionally used successfully (ref. 23). Nonetheless, the availability of these enzymes is often critical, especially when considering the appropriate regio- and stereospecificity required for a given target (ref. 22, 24 and 25).

Protein engineering based on rational, semi-rational or fully combinatorial approaches (directed evolution) has also proven to be extremely useful to generate catalysts with improved natural properties but also to create new substrate specificities (ref. 26, 27). In the field of carbohydrate-enzymes, glycosyltransferase substrate specificity has been successfully modified by site-directed mutagenesis assisted by computational modelling or directed evolution for the synthesis of biologically relevant carbohydrate structure (ref. 27). Promiscuous β-glycosidases showing altered and new specificities towards the donor or the acceptor sugar have been generated (ref. 28, 29, 31). Engineering of new glycosynthases (i.e., enzymes catalyzing the condensation of sugar residues for synthesizing a glycoside) from β-glycosidases (classified into EC 3.2.1) also emerged as a powerful way to generate modified transferases, even if they use only fluoride donors (ref 32). In addition, this methodology has never been shown to be successful for α-retaining enzyme. However, one case of active glycosynthase derived from invertase enzymes has been described up to now (ref 33).

Interestingly, the use of intermediates issued from enzymatic glycosylation in the subsequent generation of glycoconjugates of higher complexity has also been reported (ref 36-38). In all cases, the building blocks, conceived by action of native glycosyltransferases, were converted to donors and used as such, following peracylation.

The term “building block” refers to a suitably protected carbohydrate intermediate occurring in the chemical pathway of synthesis of complex oligosaccharides, e.g., said carbohydrate can be a disaccharide.

The term “intermediate” refers to a compound, protected or not, issued from an enzymatic and/or synthetic step, and involved in the multi-step synthesis of a specific target, e.g., said compound can be a disaccharide.

The design of an appropriate enzymatic glycosylation tool that would allow an optimal combination of the chemical and enzymatic steps involved in the synthesis of complex oligosaccharides has never been attempted although it would be of major interest to develop new synthetic pathways.

The Inventors have thus investigated the applicability of enzymatic glycosylation for the synthesis of building blocks compatible with chemical chain extension both at the reducing and non-reducing ends, which is compatible with subsequent conversion into donors as well as into acceptors. In the course of their investigation, the Inventors have surprisingly demonstrated the applicability of engineered amylosucrases (AS)—which are glycoside hydrolases—to the synthesis of disaccharide intermediates to synthetic oligosaccharide fragments of Shigella flexneri 1a and/or 1b lipopolysaccharide by in vitro chemo-enzymatic synthetic methodologies, implicating an enzymatic step at an early stage in the synthesis.

Amylosucrases (EC 2.4.1.4) as well as sucrose hydrolases (EC 3.2.1.-) belong to the family 13 of the glycoside hydrolases (GH13), and more particularly the subfamily 4 (GH13.4) as defined per the CAZY nomenclature (ref. 62-65). Amylosucrases and sucrose hydrolases operate on the same substrate (sucrose) with the same molecular mechanism (ref. 67). The difference between the amylosucrases and the sucrose hydrolases resides mostly in their transglycosylation abilities (ref. 65).

The structure of amylosucrase from Neisseria polysaccharea is the only known structure of enzymes from family GH13.4 (ref. 68). The single polypeptide chain (628 amino acid residues) of amylosucrase from Neisseria polysaccharea is folded into a tertiary structure consisting of five domains named N (residues 1-90), A (residues 98-184; 261-395; 461-550), B (residues 185-260), B′ (residues 395-460) and C (residues 555-628). Domains A, B and C are common domains found in family GH13. Domains N and B′ are specific to family GH13.4. Domain N is the N-terminal domain composed of 6 α-helices. Domain A is made up of eight alternating β-sheets (β1-β8) and α-helices (α1-α8) building up the catalytic core: the (β/α)₈ barrel common to family GH13. It contains also eight loops connecting helices to strands (labeled loop1 to loop8). Domain B, or loop 3, is an extension of domain A, containing two short antiparallel β-sheets flanked by two α-helices. Domain B′, or loop 7, is another extension of domain A, composed of two α-helices followed by a β-sheet and another short α-helice. Domain C is an eight-stranded β-sandwich found C-terminal to the (β/α)₈ barrel.

Unexpectedly, the Inventors have now found eleven consensus amino acid sequences to characterize glycoside hydrolases: eight consensus motifs defined hereafter (SEQ ID NO: 1; SEQ ID NO: 2; SEQ ID NO: 3; SEQ ID NO: 6; SEQ ID NO: 7; SEQ ID NO: 8; SEQ ID NO: 10; SEQ ID NO: 11) are localised in said β-sheets (6) or said loops (2) constituting domain A, two consensus motifs (SEQ ID NO: 4; SEQ ID NO: 5) are found in said domain B and one consensus motif (SEQ ID NO: 9) is found in said domain B′.

Shigella is the causal agent of shigellosis, or bacillary dysentery. In developing countries, it induces about 1 million deaths per year, most of which involve children under five years of age (ref. 39). In countries where disease is endemic, a number of S. flexneri serotypes and to a lesser extent S. sonnei are isolated, emphasizing the need for a multivalent vaccine. Noteworthy, despite numerous clinical trials (ref. 40), no vaccine is available so far. Epidemiological as well as experimental data point to the polysaccharide part, or O-antigen (O—Ag), of the bacterial lipopolysaccharide as an important virulence factor (ref. 41) and the major target of protective humoral response against reinfection (ref. 42). S. flexneri is divided into at least 14 serotypes based on known O—Ag structures. Interestingly, protein-conjugates of short synthetic oligosaccharides mimicking S. flexneri 2a O—Ag induced in mice a potent anti-O—Ag humoral immune response, which was shown to be protective against homologous challenge (ref. 43). The diversity, associated to a close resemblance in composition, of the known S. flexneri O—Ag repeating units was found of utmost interest to challenge the investigation. Indeed, except for serotype 6, all S. flexneri O—Ag repeating units share a linear tetrasaccharide backbone (ref. 41). Diversity resides in the branching pattern, which involves O-acetyl and/or α-D-glucopyranosyl decorations (ref. 41, 44). Interestingly, at least 4 different patterns of α-D-glucosylation, have been characterized for this family of bacterial polysaccharides. N-acetyl-D-glucopyranosamine residue can be implicated as branching acceptor. By way of example, serotypes 1a and 1b of S. flexneri share the α-D-glucopyranosyl-(1→4)-N-acetyl-β-D-glucopyranosaminyl (ED) branching pattern.

Within the framework of research that has led to the present invention, the Inventors have demonstrated the chemo-enzymatic synthesis of disaccharide building blocks to S. flexneri 1a and 1b serotype-specific oligosaccharides by selecting a 2-acetamido-2-deoxy-D-glucopyranoside residue as substrate acceptor, and using as enzyme a recombinant amylosucrase (an α-retaining transglucosidase from family 13 of glycoside-hydrolases that uses sucrose as glucosyl donor, (ref 45, 46)), at an earlier stage of a multi-step synthesis. New amylosucrase specificities were then surprisingly generated to glucosylate efficiently and regiospecifically allyl 2-acetamido-2-deoxy-α-D-glucopyranoside to provide building blocks compatible with chemical chain elongation as exemplified (FIGS. 2A, 2C, and 2D, Examples 2, 3, and 4).

Here-under are examples of repeating units and/or cores of bacterial surface polysaccharides containing the disaccharide motives synthesized by glucansucrases (ref 39, 41):

| Target disaccharide | Organism | Structure |
|---|---|---|
| α-D-Glcp-(1→4)-D- | E. coli O18B1 | →6)-α-D-Glcp- |
| GlcpNAc | | (1→4)-[β-D-Glcp-(1→3)]- |
| | | α-D-Galp(1→3)-[α- |
| | | D-Glcp-(1→4)]-α-D- |
| | | GlcpNAc-(1→2)-α-L-Rhap-(1→ |
| | H. pantelleriensis | →6)-α-D-Glcp-(1→4)- |
| | | α-D-GlcpNAc-(1→ |
| | | as core fragment |

Accordingly, the present invention provides a method for preparing a building block corresponding to the disaccharide α-D-glucopyranosyl-(1→4)-2-N-acetyl-2-deoxy-α-D-glucopyranoside of formula (I):

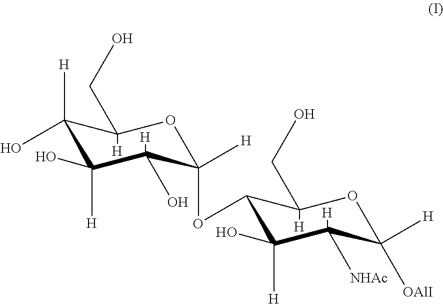

said method being characterized in that it comprises the step of using a mutant of a wild-type glycoside hydrolase, wherein said wild type glycoside hydrolase has 450 to 850 amino acids, preferably 580 to 735 amino acids, and comprises, preferably from the N- to C-terminus, eleven motifs defined by the following consensus motifs:

(1) the amino acid sequence LGVNYLHLMPL (SEQ ID NO: 1), which is located in the β-strand 2 of said wild type glycoside hydrolase;

(2) the amino acid sequence DGGYAV (SEQ ID NO: 2), which is located in the loop 2 of the (β/α)₈-barrel of said wild type glycoside hydrolase;

(3) the amino acid sequence DFVFNH (SEQ ID NO: 3) which is located in the β-strand 3 of said wild type glycoside hydrolase;

(4) the amino acid sequence LREIFPDTAPGNF (SEQ ID NO: 4), which is located in the domain B of said wild type glycoside hydrolase;

(5) the amino acid sequence FNSYQWDLN (SEQ ID NO: 5), which is located in the C-terminal part of the domain B of said wild type glycoside hydrolase;

(6) the amino acid sequence ILRLDAVAFLWK (SEQ ID NO: 6), which is located in the β-strand 4 of said wild type glycoside hydrolase;

(7) the amino acid sequence EAIV (SEQ ID NO: 7), which is located in the β-strand 5 of said wild type glycoside hydrolase;

(8) the amino acid sequence YVRCHDDI (SEQ ID NO: 8), which is located in the β-strand 7 of said wild type glycoside hydrolase;

(9) the amino acid sequence RISGTLASLAG (SEQ ID NO: 9), which is located in the domain B′ of said wild type glycoside hydrolase;

(10) the amino acid sequence GIPLIYLGDE (SEQ ID NO: 10), which is located in the β-strand 8 of said wild type glycoside hydrolase;

(11) the amino acid sequence RWVHRP (SEQ ID NO: 11), which is located in the loop 8 of the (β/α)₈-barrel,

and the sequence formed by said eleven motifs joined end-to-end from motif (1) to motif (11) of said wild type glycoside hydrolase has at least 65%, preferably at least 70%, and by order of increasing preference, at least 75%, 80%, 85%, 90%, 95%, 95%, 97%, 98%, and 99%, or 100% sequence identity or at least 80%, preferably at least 85%, and by order of increasing preference, at least 90%, 95%, 95%, 97%, 98%, and 99%, or 100% sequence similarity with the amino acid sequence SEQ ID NO: 12, which is formed by the concatenation of the eleven consensus motifs ordered from (1) to (11);

wherein said mutant has one or two mutation(s) consisting of, when said mutant has only one mutation:
the substitution of the amino acid residue at position 4 in said motif (4) with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V) with the provisio that said wild type glycoside hydrolase does not contain a valine at this position, tryptophan (W) and tyrosine (Y), or
the substitution of the amino acid residue at position 5 in said motif (4) with any amino acid selected from the group consisting of leucine (L), methionine (M) and valine (V), or
the substitution of the amino acid residue at position 8 in said motif (6) with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the amino acid residue at position 9 in said motif (6) with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), or
the substitution of the amino acid residue at position 4 in said motif (7) with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the amino acid residue at position 7 in said motif (8) with any amino acid selected from the group consisting of alanine (A) and valine (V), or
the substitution of the amino acid residue at position 1 in said motif (9) with any amino acid selected from the group consisting of alanine (A), cysteine (C), phenylalanine (F), glycine (G) with the provisio that said wild type glycoside hydrolase does not contain a glycine at this position, lysine (K), asparagine (N), glutamine (Q), serine (S) with the provisio that said wild type glycoside hydrolase does not contain a serine at this position, threonine (T) and tryptophan (W), or when said mutant has two mutations:
the substitution of the amino acid residue at position 4 in said motif (4) with an alanine (A) and the substitution of the amino acid residue at position 9 in said motif (6) with a histidine (H), or
the substitution of the amino acid residue at position 4 in said motif (4) with a cysteine (C) and the substitution of the amino acid residue at position 5 in said motif (4) with a leucine (L), or
the substitution of the amino acid residue at position 4 in said motif (4) with a lysine (K) and the substitution of the amino acid residue at position 9 in said motif (6) with any amino acid selected from the group consisting of leucine (L) and tryptophan (W), or
the substitution of the amino acid residues at positions 4 and 5 in said motif (4) respectively with a leucine (L), or
the substitution of the amino acid residues at positions 4 and 5 in said motif (4) respectively with a methionine (M), or
the substitution of the amino acid residue at position 4 in said motif (4) with a proline (P) and the substitution of the amino acid residue at position 9 in said motif (6) with a cysteine (C), or
the substitution of the amino acid residue at position 4 in said motif (4) with a threonine (T) and the substitution of the amino acid residue at position 9 in said motif (6) with any amino acid selected from the group consisting of histidine (H) and lysine (K), or
the substitution of the amino acid residue at position 4 in said motif (4) with a valine (V) and the substitution of the amino acid residue at position 5 in said motif (4) with any amino acid selected from the group consisting of leucine (L) and methionine (M), or
the substitution of the amino acid residue at position 4 in said motif (4) with a valine (V) and the substitution of the amino acid residue at position 9 in said motif (6) with any amino acid selected from the group consisting of histidine (H), lysine (K), arginine (R), and valine (V), or
the substitution of the amino acid residue at position 8 in said motif (6) with a histidine (H) and the substitution of the amino acid residue at position 9 in said motif (6) with a serine (S), or
the substitution of the amino acid residue at position 8 in said motif (6) with a proline (P) and the substitution of the amino acid residue at position 9 in said motif (6) with any amino acid selected from the group consisting of cysteine (C), isoleucine (I) and leucine (L), these mutants being preferred, or
the substitution of the amino acid residue at position 8 in said motif (6) with a threonine (T) and the substitution of the amino acid residue at position 9 in said motif (6) with a histidine (H).

According to a preferred embodiment of said mutant of a wild type glycoside hydrolase having only one mutation, the amino acid residue at position 9 in said motif (6) is substituted with any amino acid selected from the group consisting of cysteine (C), aspartic acid (D), isoleucine (I), lysine (K) and glutamine (Q), and more preferably with any amino acid selected from the group consisting of aspartic acid (D) and lysine (K).

A “wild type glycoside hydrolase” refers to an amylosucrase (EC 2.4.1.4) or a sucrose hydrolase (EC 3.2.1.-), preferably an amylosucrase. A wild type glycoside hydrolase belongs to the family 13, subfamily 4, of the glycoside hydrolases (GH13.4) as defined per the CAZY nomenclature (ref. 66-69, http://www.cazy.org).

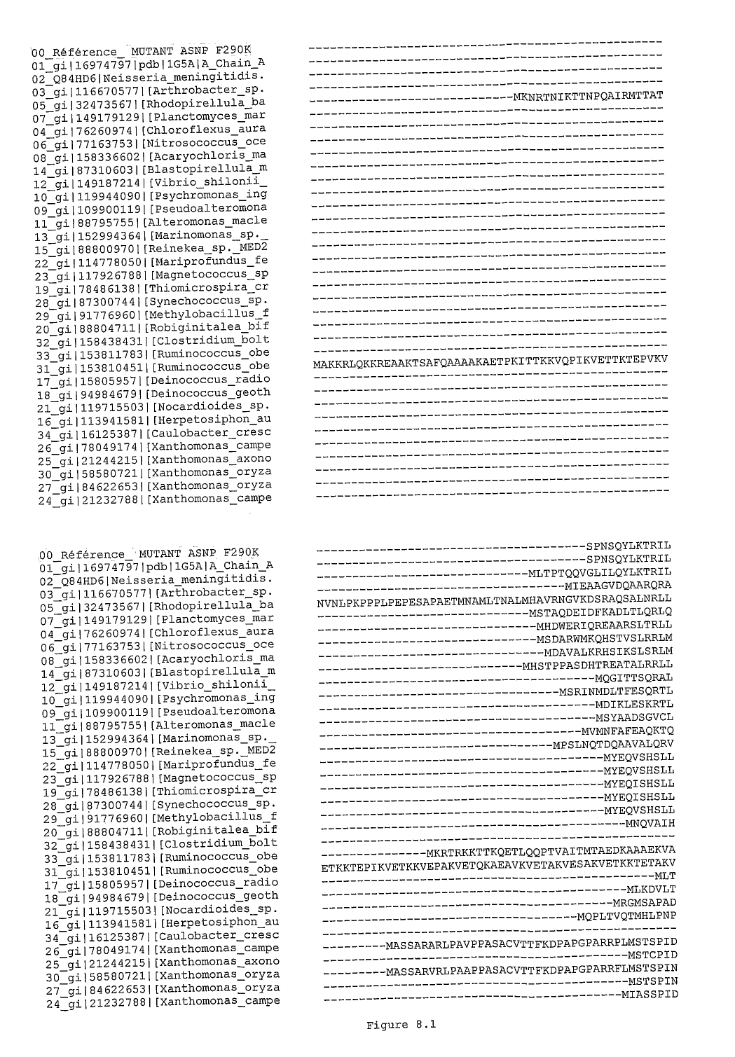

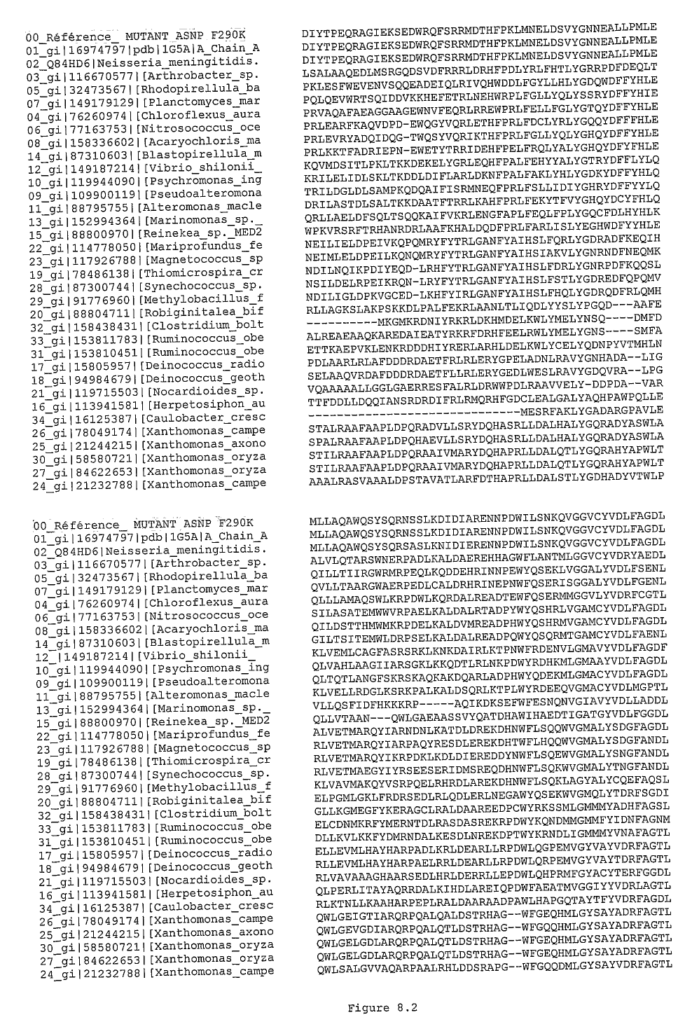

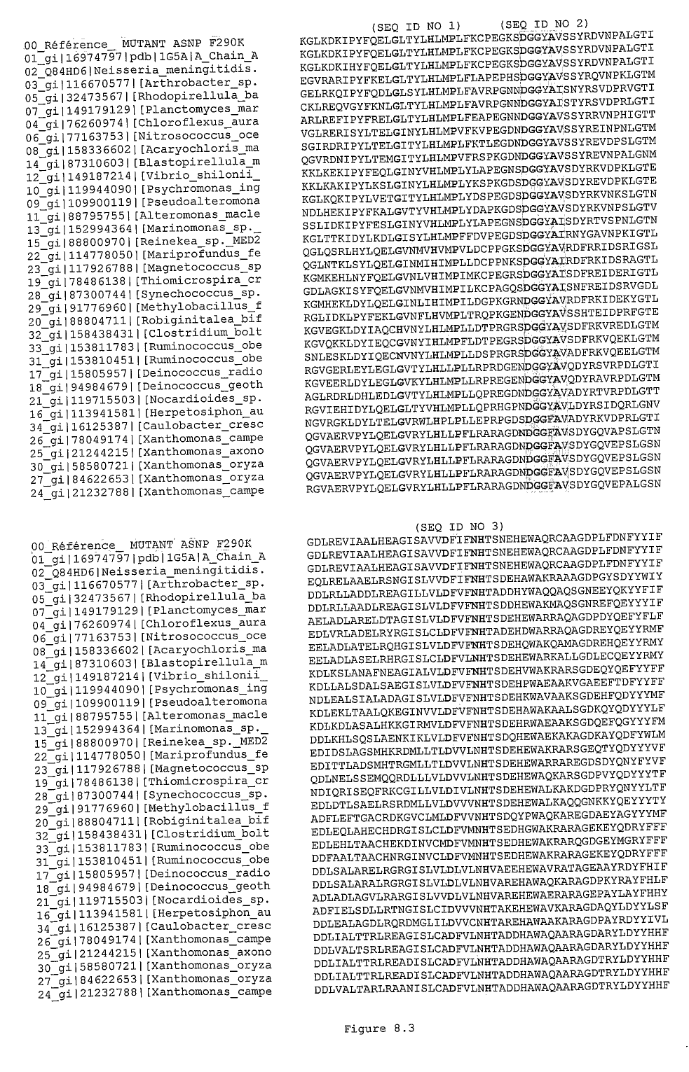

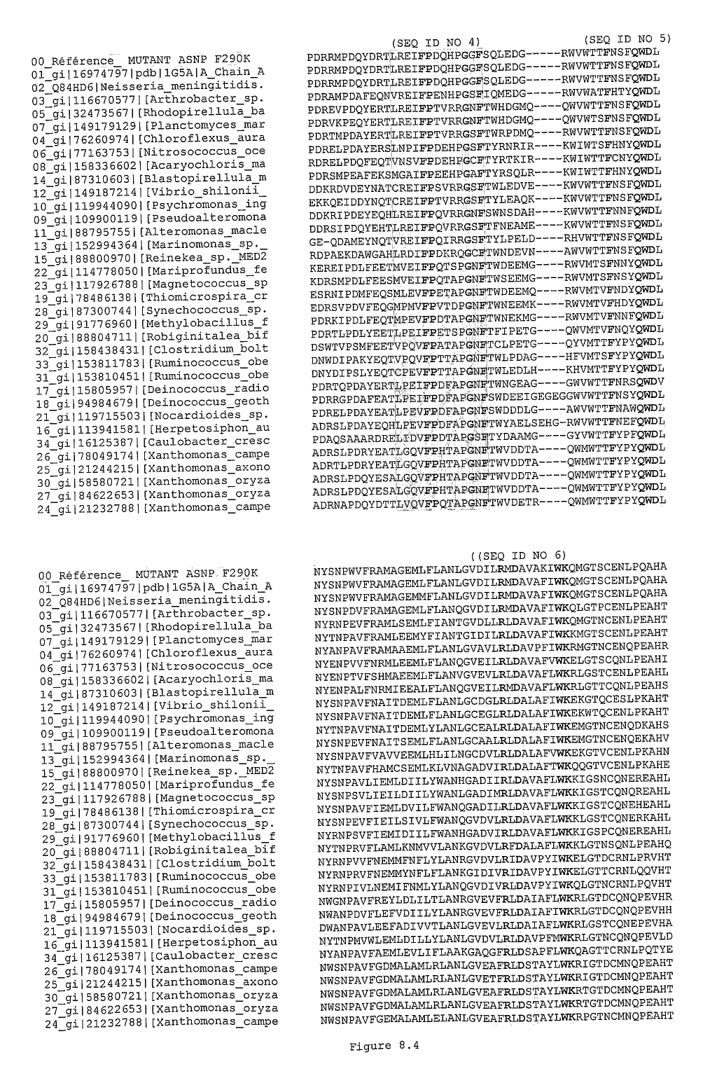

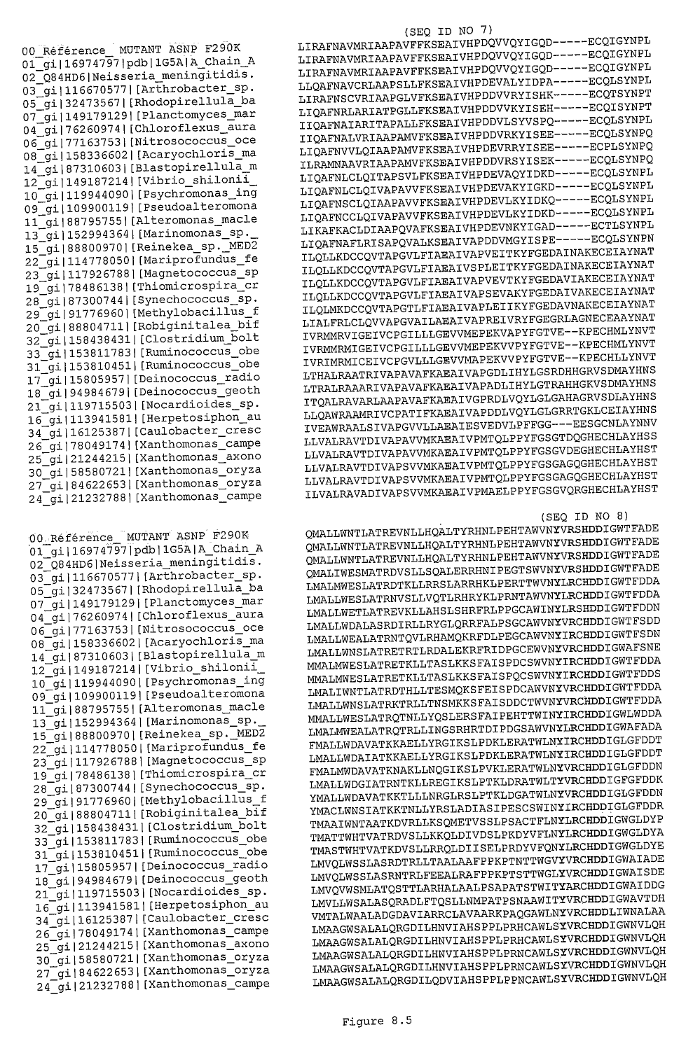

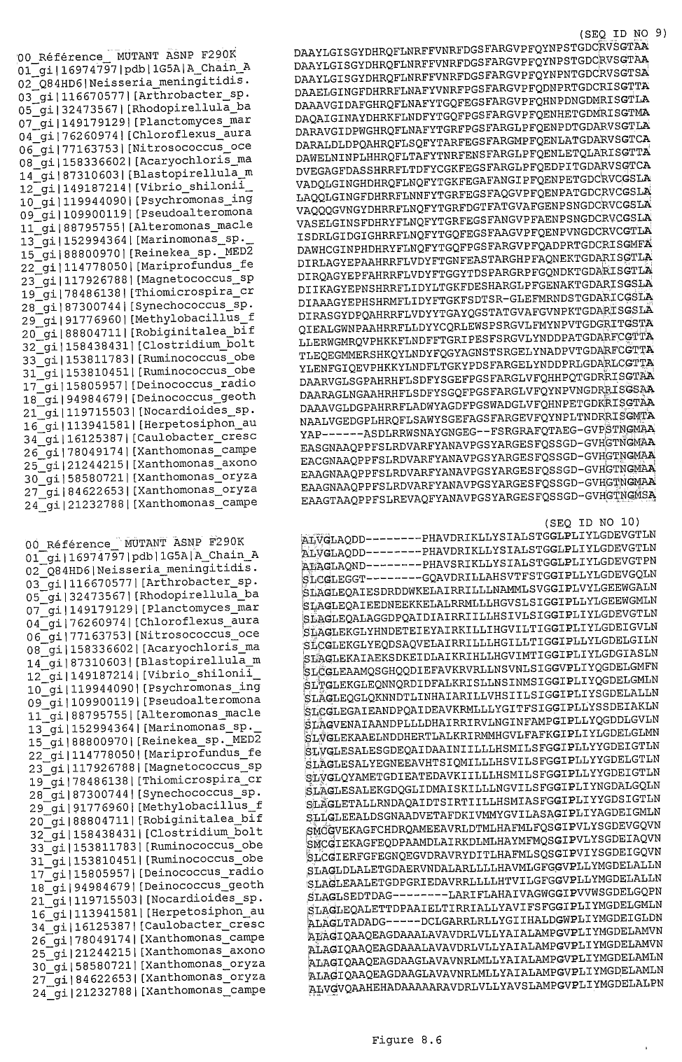

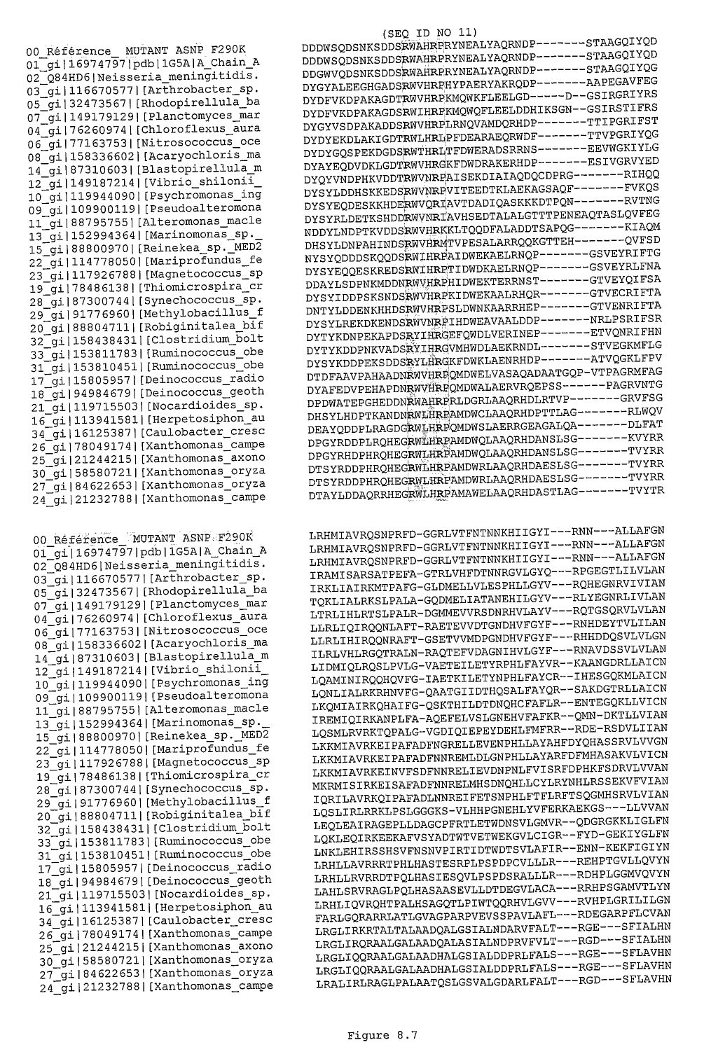

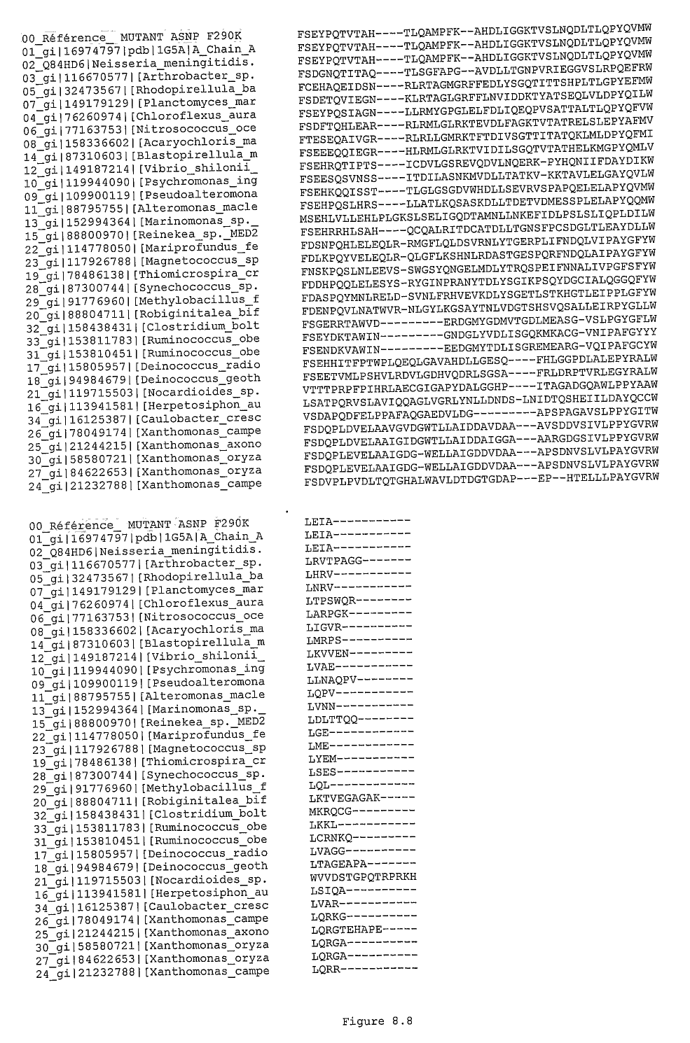

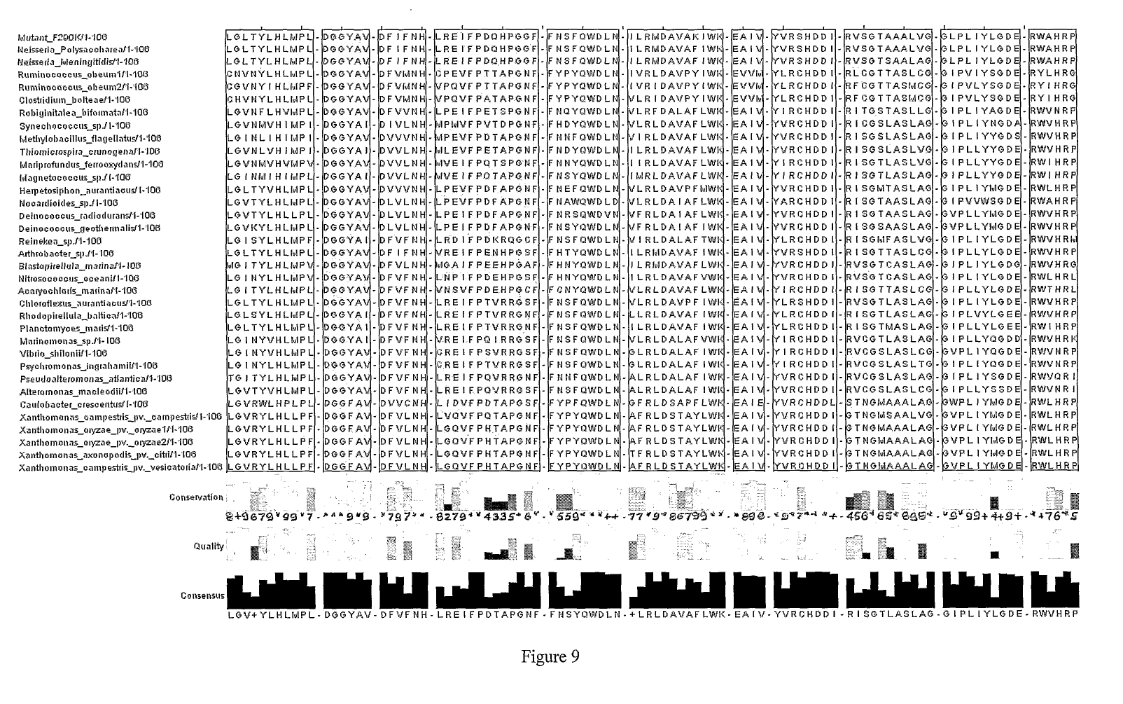

The eleven consensus motifs of the wild type glycoside hydrolases have been found by the Inventors by aligning 34 wild type glycoside hydrolases as shown in FIGS. 8 and 9.

By way of example, the glycoside hydrolase 1G5A (gi|16974797, SEQ ID NO: 13) comprises, from the N- to C-terminus, the eleven following motifs: (1) 125-134, (2) 144-149, (3) 182-187, (4) 225-237, (5) 250-258, (6) 282-293, (7) 328-331, (8) 388-395, (9) 446-456, (10) 480-489 and (11) 509-514 of SEQ ID NO: 13. These eleven motifs joined end-to-end form motif (1) to motif (11) form an amino acid sequence which has 83% sequence identity and 92% sequence similarity with the sequence SEQ ID NO: 12.

In order to identify the eleven motifs from a wild type glycoside hydrolase, one of skilled in the art can align the amino acid sequence of this wild type glycoside hydrolase against the amino acid sequence of the glycoside hydrolase 1G5A (SEQ ID NO: 13) for example, and therefore identify the eleven motifs thereof matching the eleven consensus motifs as described above.

Unless otherwise specified, sequence alignments are performed using the well-known MUSCLE program under default parameters (http://phylogenomics.berkeley.edu/cgi-bin/muscle/input_muscle.py). Jalview software can be used for visualizing the alignment and generating the eleven motifs joined end-to-end. The sequence identity and similarity values provided herein are calculated using the Vector NTI AlignX program (V9.1.0, Invitrogen, USA) on a comparison window including the whole set of eleven consensus motifs ordered from (1) to (11) as defined above.

In a particular embodiment of said wild type glycoside hydrolase, it is an amylosucrase selected from the group consisting of the proteins available in the GENBANK database under the following accession number: gi|16974797 (named 1G5A and also reproduced herein as SEQ ID NO: 13), gi|99031739 (named 1ZS2), gi|27574003 (1MVY), gi|27574004 (named 1MW0), gi|47169012 (named 1S46), gi|16974938 (named 1JGI), gi|27574006 (named 1MW2), gi|27574007 (named 1MW3), gi|27574005 (named 1MW1), gi|16974937 (named 1JG9), gi|27728142 (named Q84HD6), gi|116670577, gi|32473567, gi|149179129, gi|76260974, gi|77163753, gi|158336602, gi|87310603, gi|149187214, gi|119944090, gi|109900119, gi|88795755, gi|152994364, gi|88800970, gi|114778050, gi|117926788, gi|78486138, gi|87300744, gi|91776960, gi|88804711, gi|158438431, gi|153811783, gi|153810451, gi|15805957, gi|94984679, gi|119715503, gi|113941581, gi|16125387, gi|119477809 and gi|89092061.

In a more preferred embodiment of said wild type glycoside hydrolase, it is an amylosucrase from Neisseria polysaccharea, and is preferably selected from the group consisting of 1G5A, 1ZS2, 1MVY, 1MW0, 1S46, 1JGI, 1MW2, 1MW3, 1MW1 and 1JG9 proteins.

In another preferred embodiment of said wild type glycoside hydrolase, it is a sucrose hydrolase from Xanthomonas, and is preferably selected from the group consisting of the proteins available in the GENBANK database under the following accession number: gi|78049174, gi|21244215, gi|58580721, gi|84622653, gi|21232788.

The Table I below shows the sequence identity and similarity percent of the eleven motifs joined end-to-end for each of 34 glycoside hydrolases as described above with the sequence SEQ ID NO: 12.

TABLE I

| GI number in the | | | % |
|---|---|---|---|
| GenBank Database | Organisms | % identity | similarity |
| gi\|16974797 | Neisseria polysaccharea | 83 | 92 |
| gi\|27728142 | Neisseria meningitidis | 84 | 92 |
| gi\|116670577 | Arthrobacter sp. FB24 | 83 | 90 |
| gi\|76260974 | Chloroflexus aurantiacus J-10-fl | 85 | 93 |
| gi\|32473567 | Rhodopirellula baltica SH 1 | 86 | 91 |
| gi\|77163753 | Nitrosococcus oceani ATCC 19707 | 85 | 92 |
| gi\|149179129 | Planctomyces maris DSM 8797 | 85 | 91 |
| gi\|158336602 | Acaryochloris marina MBIC11017 | 81 | 93 |
| gi\|109900119 | Pseudoalteromonas atlantica T6c | 81 | 93 |
| gi\|119944090 | Psychromonas ingrahamii 37 | 81 | 91 |
| gi\|88795755 | Alteromonas macleodii ‘Deep ecotype’ | 79 | 90 |
| gi\|149187214 | Vibrio shilonii AK1 | 79 | 88 |
| gi\|152994364 | Marinomonas sp. MWYL1 | 79 | 86 |
| gi\|87310603 | Blastopirellula marina DSM 3645 | 80 | 91 |
| gi\|88800970 | Reinekea sp. MED297 | 80 | 91 |
| gi\|113941581 | Herpetosiphon aurantiacus ATCC 23779 | 82 | 92 |
| gi\|15805957 | Deinococcus radiodurans R1 | 83 | 93 |
| gi\|94984679 | Deinococcus geothermalis DSM 11300 | 85 | 93 |
| gi\|78486138 | Thiomicrospira crunogena XCL-2 | 83 | 91 |
| gi\|88804711 | Robiginitalea biformata HTCC2501 | 82 | 90 |
| gi\|119715503 | Nocardioides sp. JS614 | 81 | 91 |
| gi\|114778050 | Mariprofundus ferrooxydans PV-1 | 82 | 91 |
| gi\|117926788 | Magnetococcus sp. MC-1 | 84 | 91 |
| gi\|21232788 | Xanthomonas campestris pv. campestris | 73 | 88 |
| | str. | | |
| gi\|21244215 | Xanthomonas axonopodis pv. citri str. 306 | 74 | 88 |
| gi\|78049174 | Xanthomonas campestris pv. vesicatoria | 74 | 88 |
| | str. 85-10 | | |
| gi\|84622653 | Xanthomonas oryzae pv. oryzae MAFF | 74 | 88 |
| | 311018 | | |
| gi\|87300744 | Synechococcus sp. WH 5701 | 79 | 91 |
| gi\|91776960 | Methylobacillus flagellatus KT | 82 | 88 |
| gi\|58580721 | Xanthomonas oryzae pv. oryzae | 74 | 88 |
| | KACC10331 | | |
| gi\|153810451 | Ruminococcus obeum ATCC 29174 | 73 | 88 |
| gi\|158438431 | Clostridium bolteae ATCC BAA-613 | 68 | 84 |
| gi\|153811783 | Ruminococcus obeum ATCC 29174 | 67 | 83 |
| gi\|16125387 | Caulobacter crescentus CB15 | 68 | 86 |

According to a preferred embodiment of said wild type glycoside hydrolase, it contains an isoleucine (I) or valine (V) residue at position 4 in said motif (4), preferably an isoleucine.

According to another preferred embodiment of said wild type glycoside hydrolase, it contains a phenylalanine (F) residue at position 5 in said motif (4).

According to another preferred embodiment of said wild type glycoside hydrolase, it contains an alanine (A) or proline (P) residue at position 8 in said motif (6), preferably an alanine.

According to another preferred embodiment of said wild type glycoside hydrolase, it contains a phenylalanine (F) or a tyrosine (Y) residue at position 9 in said motif (6), preferably a phenylalanine.

According to another preferred embodiment of the said wild type glycoside hydrolase, it contains a valine (V), a methionine (M) or a glutamic acid (E) residue at position 4 in said motif (7), preferably a valine.

According to another preferred embodiment of said wild-type glycoside hydrolase, it contains an aspartic acid (D) residue at position 7 in said motif (8).

According to another preferred embodiment of said wild-type glycoside hydrolase, it contains a glycine (G), an arginine (R) or a serine (S) residue at position 1 in said motif (9), preferably a glycine.

In a preferred embodiment of said mutant of a glycoside hydrolase, it is a mutant of the 1G5A amylosucrase from Neisseria polysaccharea having the amino acid sequence SEQ ID NO: 13, wherein said mutant, has in reference to SEQ ID NO: 13, a mutation consisting of,

when said mutant has only one mutation:
the substitution of the isoleucine (I) residue at position 228 (I228), corresponding to position 4 in said motif (4), with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V) with the provisio that said wild type glycoside hydrolase does not contain a valine at this position, tryptophan (W) and tyrosine (Y), or
the substitution of the phenylalanine (F) at position 229 (F229) corresponding to position 5 in said motif (4), with any amino acid selected from the group consisting of leucine (L), methionine (M) and valine (V), or
the substitution of the alanine (A) residue at position 289 (A289), corresponding to position 8 in said motif (6), with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the phenylalanine (F) residue at position 290 (F290), corresponding to position 9 in said motif (6), with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), preferably with any amino acid selected from the group consisting of cysteine (C), aspartic acid (D), isoleucine (I), lysine (K) and glutamine (Q), and more preferably with any amino acid selected from the group consisting of aspartic acid (D) and lysine (K), or
the substitution of the valine (V) residue at position 331 (V331), corresponding to position 4 in said motif (7), with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the aspartic acid (D) residue at position 394 (D394), corresponding to position 7 in said motif (8), with any amino acid selected from the group consisting of alanine (A) and valine (V), or
the substitution of the arginine (R) residue at position 446 (R446), corresponding to position 1 in said motif (9), with any amino acid selected from the group consisting of cysteine (C), phenylalanine (F), glycine (G), lysine (K), asparagine (N), glutamine (Q), serine (S), threonine (T) and tryptophan (W), or when said mutant has two mutations:
the substitution of the isoleucine (I) residue at position 228 (I228), corresponding to position 4 in said motif (4), with an alanine (A) and the substitution of the phenylalanine (F) residue at position 290 (F290), corresponding to position 9 in said motif (6), with a histidine (H), or
the substitution of the isoleucine (I) residue at position 228 (I228), corresponding position 4 in said motif (4), with a cysteine (C) and the substitution of the phenylalanine (F) residue at position 229 (F229), corresponding to position 5 in said motif (4), with a leucine (L), or
the substitution of the isoleucine (I) residue at position 228 (I228), corresponding position 4 in said motif (4), with a lysine (K) and the substitution of the phenylalanine (F) residue at position 290 (F290), corresponding to position 9 in said motif (6), with any amino acid selected from the group consisting of leucine (L) and tryptophan (W), or
the substitution of the isoleucine (I) residue at position 228 (I228), corresponding to position 4 in said motif (4), with a leucine (L) and the substitution of the phenylalanine (F) residue at position 229 (F229), corresponding to position 5 in said motif (4) with a leucine (L), or
the substitution of the isoleucine (I) residue at position 228 (I228), corresponding to position 4 in said motif (4), with a methionine (M) and the substitution of the phenylalanine (F) residue at position 229 (F229), corresponding to position 5 in said motif (4), with a methionine (M), or
the substitution of the isoleucine (I) residue at position 228 (I228), corresponding to position 4 in said motif (4), with proline (P) and the substitution of the phenylalanine (F) residue at position 290 (F290), corresponding to position 9 in said motif (6), with a cysteine (C), or
the substitution of the isoleucine (I) residue at position 228 (I228), corresponding to position 4 in said motif (4), with a threonine (T) and the substitution of the phenylalanine (F) residue at position 290 (F290), corresponding to position 9 in said motif (6), with any amino acid selected from the group consisting of histidine (H) and lysine (K), or
the substitution of the isoleucine (I) residue at position 228 (I228), corresponding to position 4 in said motif (4), with a valine (V) and the substitution of the phenylalanine (F) residue at position 229 (F229), corresponding to position 5 in said motif (4), with any amino acid selected from the group consisting of leucine (L) and methionine (M), or
the substitution of the isoleucine (I) residue at position 228 (I228), corresponding to position 4 in said motif (4), with a valine (V) and the substitution of the phenylalanine (F) residue at position 290 (F290), corresponding to position 9 in said motif (6), with any amino acid selected from the group consisting of histidine (H), lysine (K), arginine (R), and valine (V), or
the substitution of the alanine (A) residue at position 289 (A289), corresponding to position 8 in said motif (6), with a histidine (H) and the substitution of the phenylalanine (F) residue at position 290 (F290), corresponding to position 9 in said motif (6), with a serine (S), or
the substitution of the alanine (A) residue at position 289 (A289), corresponding to position 8 in said motif (6), with a proline (P) and the substitution of the phenylalanine (F) residue at position 290 (F290), corresponding to position 9 in said motif (6), with any amino acid selected from the group consisting of cysteine (C), isoleucine (I) and leucine (L), or
the substitution of the alanine (A) residue at position 289 (A289), corresponding to position 8 in said motif (6), with a threonine (T) and the substitution of the phenylalanine (F) residue at position 290 (F290), corresponding to position 9 in said motif (6), with a histidine (H).Unexpectedly, the mutants of a glycoside hydrolase according to the present invention present a specific activity toward D-GlcpNHTCA (NHTCA is N-trichloroacetyl) or/and substantially improve the glucosylation rate of D-GlcpNAc and α-D-GlcpNAc-OAll. The use of an appropriate combination of a mutant of a glycoside hydrolase of the present invention with a donor and an acceptor as defined in the present invention at an earlier stage of a multi-step synthesis leads to the synthesis of complex oligosaccharides, such as S. flexneri 1a and 1b O-antigens.

Especially, the invention is directed to a method for preparing the building block corresponding to a disaccharide α-D-glucopyranosyl-(1→44)-2-amino-2-deoxy-α-D-glucopyranoside of formula and/or α-D-glucopyranosyl-(1→4)-2-N-acyl-2-deoxy-α-D-glucopyranoside of formula (Ia):

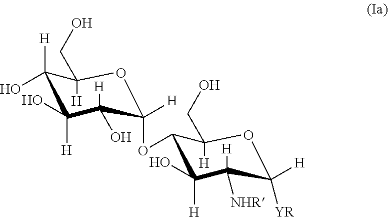

advantageously the disaccharide allyl α-D-glucopyranosyl-(1→4)-2-N-acetyl-2-deoxy-α-D-glucopyranoside of formula (I):

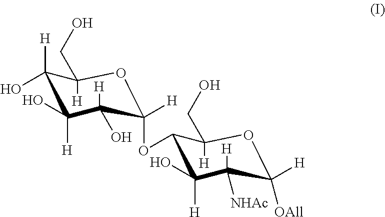

said method being characterized in that it comprises the step of reacting a mutant of a glycoside hydrolase as above disclosed, with the acceptor of formula (II), preferably of formula (IIa):

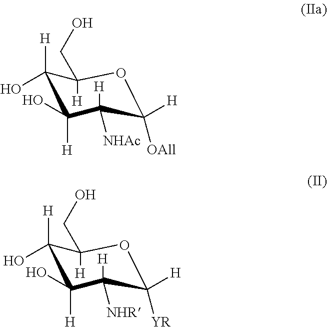

wherein Y is selected from —O— and —S— and R is selected from the group consisting of: C₁-C₆ alkyl, C₁-C₆ alkenyl, aryl, allyl, —CO-alkyl (C₁-C₆), —CO-alkenyl (C₁-C₆), —CO-aryl,

R′ designates a group selected from: acetyl, trichloroacetyl (TCA), trifluoroacetyl (TFA),

wherein aryl designates an aromatic group like phenyl, benzyl, possibly substituted by one or several of the following groups: C₁-C₄ alkyl, —NO₂, a halogen atom, —O-alkyl (C₁-C₆),

with a donor of formula (IIIa):

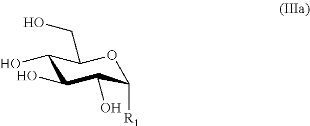

wherein R₁ represents a group selected from:

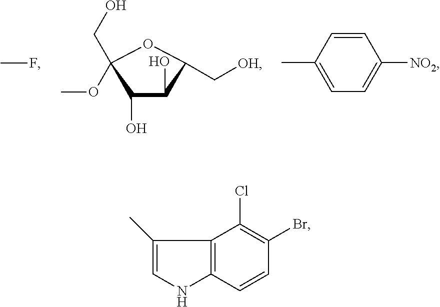

and preferably with the donor of formula (III), sucrose:

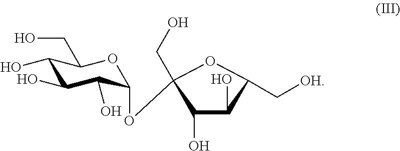

Another object of the invention is a method for the preparation of the building block corresponding to the disaccharide of formula (XX_{3B}) in which R₂ represents a group selected from H, Bn, Ac and AcBn and R₃ represents a group selected from H and Ac.

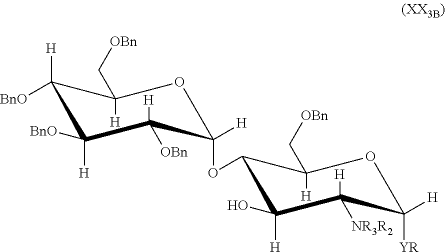

and preferably corresponding to the disaccharide of formula (XX_{3A})

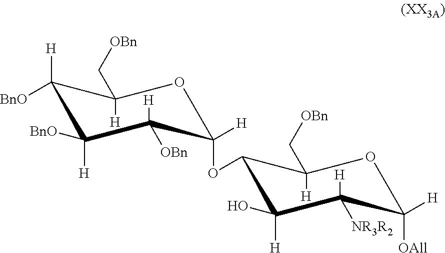

comprising at least one step wherein the acceptor of formula (IIa), advantageously (II), is reacted with a mutant of a glycoside hydrolase as above disclosed to give the disaccharide of formula (Ia)

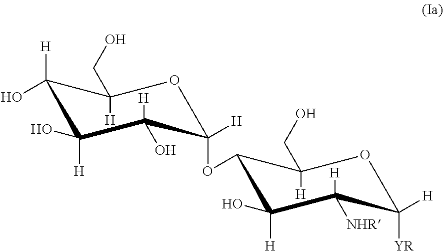

advantageously of formula (I)

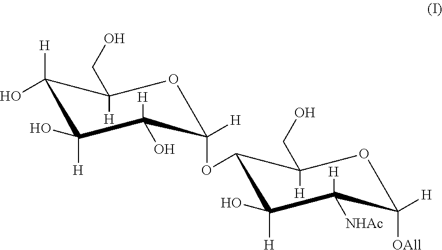

Some of the molecules obtained by the method of the invention are new and as such are another object of the invention. Their list is given here-under:
Allyl α-D-glucopyranosyl-(1→4)-α-D-glucopyranosyl-(1→4)-2-acetamido-2-deoxy-α-d-glucopyranoside (XX₂),
Allyl α-D-glucopyranosyl-(1→4)-2-acetamido-2-deoxy-α-D-glucopyranoside (XX₃),
Allyl 2,3,4,6-tetra-O-acetyl-α-D-glucopyranosyl-(1→4)-2-acetamido-3,6-di-O-acetyl-2-deoxy-α-D-glucopyranoside (XX₄),
Allyl α-D-glucopyranosyl-(1→4)-2-amino-2-deoxy-α-D-glucopyranoside (XX₅),
Allyl α-D-glucopyranosyl-(1→4)-2-amino-2-N,3-O-carbonyl-2-deoxy-α-D-glucopyranoside (XX₆),
Allyl 2,4,6-tri-O-benzyl-α-D-glucopyranosyl-(1→4)-6-O-benzyl-2-benzylamino-2-N,3-O-carbonyl-2-deoxy-α-D-glucopyranoside (XX₇),
Allyl 2,3,4,6-tetra-O-benzyl-α-D-glucopyranosyl-(1→4)-6-O-benzyl-2-benzylamino-2-N,3-O-carbonyl-2-deoxy-α-D-glucopyranoside (XX₈),
Allyl 2,3,4,6-tetra-O-benzyl-α-D-glucopyranosyl-(1→4)-6-O-benzyl-2-amino-2-N,3-O-carbonyl-2-deoxy-α-D-glucopyranoside (XX_{8A}),
Allyl 2,3,4,6-tetra-O-benzyl-α-D-glucopyranosyl-(1→4)-6-O-benzyl-2-benzylamino-2-deoxy-α-D-glucopyranoside (XX₉),
Allyl 2,3,4,6-tetra-O-benzyl-α-D-glucopyranosyl-(1→4)-6-O-benzyl-2-benzylacetamido-2-deoxy-α-D-glucopyranoside (XX₁₀),
Allyl 2-O-benzoyl-4-O-benzyl-3-O-chloro acetyl-α-L-rhamnopyranosyl-(1→3)-[2,3,4,6-tetra-O-benzyl-α-D-glucopyranosyl-(1→4)]-6-O-benzyl-2-benzylacetamido-2-deoxy-α-D-glucopyranoside (XX₁₁),
Allyl 2-O-acetyl-3,4-di-O-benzyl-α-L-rhamnopyranosyl-(1→3)-[2,3,4,6-tetra-O-benzyl-α-D-glucopyranosyl-(1→4)]-6-O-benzyl-2-benzylacetamido-2-deoxy-α-D-glucopyrano side (XX₁₂),
Allyl α-D-glucopyranosyl-(1→4)-2-deoxy-2-trichloroacetamido-α-D-glucopyrano side (XX₁₃),
Allyl 2,3,4,6-tetra-O-acetyl-α-D-glucopyranosyl-(1→4)-3,6-di-O-acetyl-2-deoxy-2-trichloroacetamido-α-D-glucopyranoside (XX₁₄),
2,3,4,6-tetra-O-acetyl-α-D-glucopyranosyl-(1→4)-3,6-di-O-acetyl-2-deoxy-2-trichloroacetamido-α-D-glucopyranose (XX₁₅),
2,3,4,6-Tetra-O-acetyl-α-D-glucopyranosyl-(1→4)-3,6-di-O-acetyl-2-deoxy-2-trichloroacetamido-α-D-glucopyranosyl trichloroacetimidate (XX₁₆),
Allyl 2,3,4,6-tetra-O-acetyl-α-D-glucopyranosyl-(1→4)-3,6-di-O-acetyl-2-deoxy-2-trichloroacetamido-α-D-glucopyranosyl-(1→2)-3,4-di-O-benzyl-α-L-rhamnopyranoside (XX₁₇),
Allyl 2,3,4,6-tetra-O-benzyl-α-D-glucopyranosyl-(1→4)-2-acetamido-3-O-acetyl-6-O-benzyl-2-deoxy-α-D-glucopyranoside (XX₂₄),
Allyl 2,3,4,6-tetra-O-benzyl-α-D-glucopyranosyl-(1→4)-2-acetamido-6-O-benzyl-2-deoxy-α-D-glucopyranoside (XX₂₅).

Another object of the invention is a mutant of a wild-type glycoside hydrolase, said wild-type glycoside hydrolase being defined as above, and said mutant having one or two mutation(s) consisting of,

when said mutant has only one mutation:
the substitution of the amino acid residue at position 4 in said motif (4) with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V) with the provisio that said wild type glycoside hydrolase does not contain a valine at this position, tryptophan (W) and tyrosine (Y), or
the substitution of the amino acid residue at position 5 in said motif (4) with any amino acid selected from the group consisting of leucine (L), methionine (M) and valine (V), or
the substitution of the amino acid residue at position 8 in said motif (6) with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the amino acid residue at position 9 in said motif (6) with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), preferably selected from the group consisting of cysteine (C), aspartic acid (D), isoleucine (I), lysine (K) and glutamine (Q), and more preferably with any amino acid selected from the group consisting of aspartic acid (D) and lysine (K), or
the substitution of the amino acid residue at position 4 in said motif (7) with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the amino acid residue at position 7 in said motif (8) with a valine (V), or
the substitution of the amino acid residue at position 1 in said motif (9) with any amino acid selected from the group consisting of alanine (A), cysteine (C), phenylalanine (F), glycine (G) with the provisio that said wild type glycoside hydrolase does not contain a glycine at this position, lysine (K), asparagine (N), glutamine (Q), serine (S) with the provisio that said wild type glycoside hydrolase does not contain a serine at this position, threonine (T) and tryptophan (W), or when said mutant has two mutations:
the substitution of the amino acid residue at position 4 in said motif (4) with an alanine (A) and the substitution of the amino acid residue at position 9 in said motif (6) with a histidine (H), or
the substitution of the amino acid residue at position 4 in said motif (4) with a cysteine (C) and the substitution of the amino acid residue at position 5 in said motif (4) with a leucine (L), or
the substitution of the amino acid residue at position 4 in said motif (4) with a lysine (K) and the substitution of the amino acid residue at position 9 in said motif (6) with any amino acid selected from the group consisting of leucine (L) and tryptophan (W), or
the substitution of the amino acid residues at positions 4 and 5 in said motif (4) respectively with a leucine (L), or
the substitution of the amino acid residues at positions 4 and 5 in said motif (4) respectively with a methionine (M), or
the substitution of the amino acid residue at position 4 in said motif (4) with a proline (P) and the substitution of the amino acid residue at position 9 in said motif (6) with a cysteine (C), or
the substitution of the amino acid residue at position 4 in said motif (4) with a threonine (T) and the substitution of the amino acid residue at position 9 in said motif (6) with any amino acid selected from the group consisting of histidine (H) and lysine (K), or
the substitution of the amino acid residue at position 4 in said motif (4) with a valine (V) and the substitution of the amino acid residue at position 5 in said motif (4) with any amino acid selected from the group consisting of leucine (L) and methionine (M), or
the substitution of the amino acid residue at position 4 in said motif (4) with a valine (V) and the substitution of the amino acid residue at position 9 in said motif (6) with any amino acid selected from the group consisting of histidine (H), lysine (K), arginine (R), and valine (V), or
the substitution of the amino acid residue at position 8 in said motif (6) with a histidine (H) and the substitution of the amino acid residue at position 9 in said motif (6) with a serine (S), or
the substitution of the amino acid residue at position 8 in said motif (6) with a proline (P) and the substitution of the amino acid residue at position 9 in said motif (6) with any amino acid selected from the group consisting of cysteine (C), isoleucine (I) and leucine (L), or
the substitution of the amino acid residue at position 8 in said motif (6) with a threonine (T) and the substitution of the amino acid residue at position 9 in said motif (6) with a histidine (H).

Advantageously, the mutants of a glycoside hydrolase according to the present invention having a mutation consisting of:
the substitution of the amino acid residue at position 4 in said motif (4) with any amino acid selected from the group consisting of alanine (A), cysteine (C), glycine (G), histidine (H), asparagine (N), serine (S), threonine (T), tryptophan (W) and tyrosine (Y), or
the substitution of the amino acid residue at position 9 in said motif (6) with any amino acid selected from the group consisting of cysteine (C), aspartic acid (D), glutamic acid (E), isoleucine (I) and valine (V),

present also a specific activity toward L-Rhap, α-L-Rhap-OMe and α-L-Rhap-OAllyl. These mutants also catalyze the glucosylation of α-L-Rhap-OMe to give the disaccharide [α-D-Glcp(1→3)]-α-L-Rhap-OMe.

In a preferred embodiment of said mutant of a wild-type glycoside hydrolase, it is a mutant of the 1G5A amylosucrase from Neisseria polysaccharea having the amino acid sequence SEQ ID NO: 13, wherein said mutant, has in reference to SEQ ID NO: 13, a mutation consisting of when said mutant has only one mutation:
the substitution of the isoleucine (I) residue at position 228 (I228) with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V), tryptophan (W) and tyrosine (Y), or
the substitution of the phenylalanine (F) at position 229 (F229) corresponding to position 5 in said motif (4), with any amino acid selected from the group consisting of leucine (L), methionine (M) and valine (V), or
the substitution of the alanine (A) residue at position 289 (A289) with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the phenylalanine (F) residue at position 290 (F290) with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), or
the substitution of the valine (V) residue at position 331 (V331) with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the aspartic acid (D) residue at position 394 (D394) with a valine (V), or
the substitution of the arginine (R) residue at position 446 (R446) with any amino acid selected from the group consisting of cysteine (C), phenylalanine (F), glycine (G), lysine (K), asparagine (N), glutamine (Q), serine (S), threonine (T) and tryptophan (W),
   when said mutant has two mutations:
the substitution of the isoleucine (I) residue at position 228 (I228), corresponding to position 4 in said motif (4), with an alanine (A) and the substitution of the phenylalanine (F) residue at position 290 (F290), corresponding to position 9 in said motif (6), with a histidine (H), or the substitution of the isoleucine (I) residue at position 228 (I228), corresponding position 4 in said motif (4), with a cysteine (C) and the substitution of the phenylalanine (F) residue at position 229 (F229), corresponding to position 5 in said motif (4), with a leucine (L), or
the substitution of the isoleucine (I) residue at position 228 (I228), corresponding position 4 in said motif (4), with a lysine (K) and the substitution of the phenylalanine (F) residue at position 290 (F290), corresponding to position 9 in said motif (6), with any amino acid selected from the group consisting of leucine (L) and tryptophan (W), or
the substitution of the isoleucine (I) residue at position 228 (I228), corresponding to position 4 in said motif (4), with a leucine (L) and the substitution of the phenylalanine (F) residue at position 229 (F229), corresponding to position 5 in said motif (4) with a leucine (L), or
the substitution of the isoleucine (I) residue at position 228 (I228), corresponding to position 4 in said motif (4), with a methionine (M) and the substitution of the phenylalanine (F) residue at position 229 (F229), corresponding to position 5 in said motif (4), with a methionine (M), or
the substitution of the isoleucine (I) residue at position 228 (I228), corresponding to position 4 in said motif (4), with proline (P) and the substitution of the phenylalanine (F) residue at position 290 (F290), corresponding to position 9 in said motif (6), with a cysteine (C), or
the substitution of the isoleucine (I) residue at position 228 (I228), corresponding to position 4 in said motif (4), with a threonine (T) and the substitution of the phenylalanine (F) residue at position 290 (F290), corresponding to position 9 in said motif (6), with any amino acid selected from the group consisting of histidine (H) and lysine (K), or
the substitution of the isoleucine (I) residue at position 228 (I228), corresponding to position 4 in said motif (4), with a valine (V) and the substitution of the phenylalanine (F) residue at position 229 (F229), corresponding to position 5 in said motif (4), with any amino acid selected from the group consisting of leucine (L) and methionine (M), or
the substitution of the isoleucine (I) residue at position 228 (I228), corresponding to position 4 in said motif (4), with a valine (V) and the substitution of the phenylalanine (F) residue at position 290 (F290), corresponding to position 9 in said motif (6), with any amino acid selected from the group consisting of histidine (H), lysine (K), arginine (R), and valine (V), or
the substitution of the alanine (A) residue at position 289 (A289), corresponding to position 8 in said motif (6), with a histidine (H) and the substitution of the phenylalanine (F) residue at position 290 (F290), corresponding to position 9 in said motif (6), with a serine (S), or
the substitution of the alanine (A) residue at position 289 (A289), corresponding to position 8 in said motif (6), with a proline (P) and the substitution of the phenylalanine (F) residue at position 290 (F290), corresponding to position 9 in said motif (6), with any amino acid selected from the group consisting of cysteine (C), isoleucine (I) and leucine (L); these mutants being preferred because they present a glucosylation rate of D-GlcpNAc of 100%, or
the substitution of the alanine (A) residue at position 289 (A289), corresponding to position 8 in said motif (6), with a threonine (T) and the substitution of the phenylalanine (F) residue at position 290 (F290), corresponding to position 9 in said motif (6), with a histidine (H).

According to a preferred embodiment of said mutant of a glycoside hydrolase, it is a mutant of the 1 G5A amylosucrase from Neisseria polysaccharea which has in reference to SEQ ID NO: 13, a mutation consisting of the substitution of the phenylalanine (F) residue at position 290 (F290) with any amino acid selected from the group consisting of cysteine (C), aspartic acid (D), isoleucine (I), lysine (K) and glutamine (Q), and more preferably with any amino acid selected from the group consisting of aspartic acid (D) and lysine (K). These mutants present a glucosylation rate of D-GlcpNAc of higher than 50%.

The present invention also provides polynucleotides encoding a mutant of a glycoside hydrolase according to the present invention.

Polynucleotides of the invention may be obtained by the well-known methods of recombinant DNA technology and/or of chemical DNA synthesis. These methods also allow introducing the desired mutations in a naturally occurring DNA sequence.

The invention also provides recombinant DNA constructs comprising a polynucleotide of the invention, such as expression cassettes wherein said polynucleotide is linked to appropriate control sequences allowing the regulation of its transcription and translation in a host cell and optionally to a sequence encoding a GST tag allowing a rapid purification of the mutant enzymes and recombinant vectors comprising a polynucleotide or an expression cassette of the invention.

Another object of the invention is a method for determining whether a wild type protein is a wild type glycoside hydrolase, said method comprising the steps of:

a) determining the amino acid sequence of said protein,

b) identifying in the amino acid sequence of said protein, preferably from the N- to C-terminus, eleven motifs defined by the following consensus motifs:

(SEQ ID NO: 1)

| | |
|---|---|
| (1) | the amino acid sequence LGVNYLHLMPL; |

(SEQ ID NO: 2)

| | |
|---|---|
| (2) | the amino acid sequence DGGYAV; |

(SEQ ID NO: 3)

| | |
|---|---|
| (3) | the amino acid sequence DFVFNH; |

(SEQ ID NO: 4)

| | |
|---|---|
| (4) | the amino acid sequence LREIFPDTAPGNF; |

(SEQ ID NO: 5)

| | |
|---|---|
| (5) | the amino acid sequence FNSYQWDLN; |

(SEQ ID NO: 6)

| | |
|---|---|
| (6) | the amino acid sequence ILRLDAVAFLWK; |

(SEQ ID NO: 7)

| | |
|---|---|
| (7) | the amino acid sequence EAIV; |

(SEQ ID NO: 8)

| | |
|---|---|
| (8) | the amino acid sequence YVRCHDDI; |

(SEQ ID NO: 9)

| | |
|---|---|
| (9) | the amino acid sequence RISGTLASLAG; |

(SEQ ID NO: 10)

| | |
|---|---|
| (10) | the amino acid sequence GIPLIYLGDE; |

(SEQ ID NO: 11)

| | |
|---|---|
| (11) | the amino acid sequence RWVHRP; |

c) determining the sequence identity percent or sequence similarity percent between the sequence formed by said eleven motifs joined end-to-end from motif (1) to motif (11) with the amino acid sequence SEQ ID NO: 12, and if the sequence identity percent is at least 65%, preferably at least 70%, and by order of increasing preference, at least 75%, 80%, 85%, 90%, 95%, 95%, 97%, 98%, and 99%, or 100% or if the sequence similarity is at least 80%, preferably at least 85%, and by order of increasing preference, at least 90%, 95%, 95%, 97%, 98%, and 99%, or 100%, then the wild type protein is a wild type glycoside hydrolase.

In addition to the preceding features, the invention further comprises other features which will emerge from the following description, which refers to examples illustrating the present invention, as well as to the appended figures.

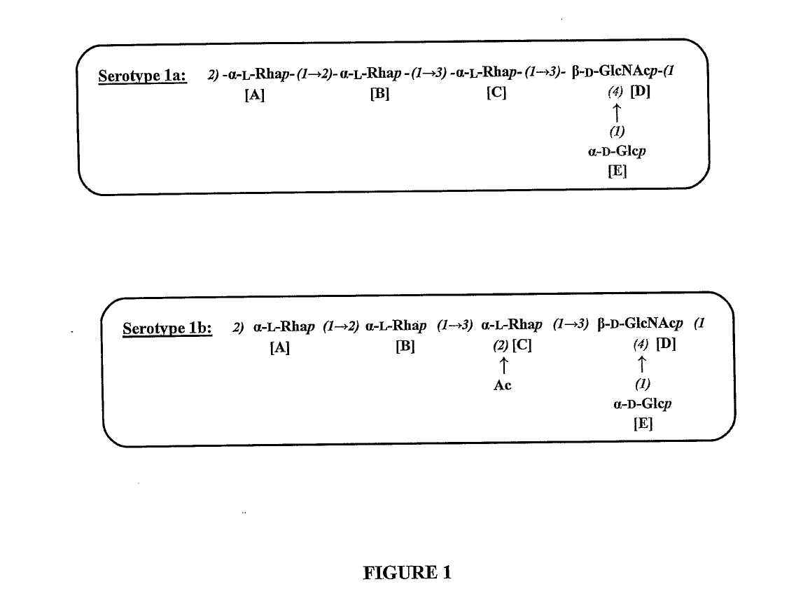

FIG. 1 shows the repeating unit of S. flexneri serotypes 1a and 1b O-Antigen. Rhap=rhamnopyranosyl—GlcpNAc=2-N-acetyl-2-deoxy-glucopyranosyl—Glcp=glucopyranosyl—Ac=acetyl.

FIG. 2 shows the first steps of chemo-enzymatic routes (A, C, D) to potential synthetic intermediates to oligosaccharide fragments of S. flexneri 1b and/or 1a O-antigens, a chemical synthetic route (B) to S. flexneri 1a pentasaccharides (ref. 66), and the chemical synthesis (E) of a model disaccharide intermediate to oligosaccharide fragments of S. flexneri 1b and/or 1a O-antigens. In FIG. 2A: Chemo-enzymatic synthesis of disaccharide XX₃. a. AllOH, BF₃.OEt₂; b. non-purified F290K extract, sucrose; c. Ac₂O, Pyridine; d. MeONa, MeOH; e. Amyloglucosidase from Aspergillus niger, acetate buffer (pH 4.8). In FIG. 2C: a. MeONa, MeOH; b. Ba(OH)₂. 8H₂O, H₂O, 90° C.; c. pNO₂C₆H₄OCOCl, MeONa, MeOH; d. (i) CCl₃CH₂OCOCl, NaOMe, (ii) BnBr, NaH; e. (i) BnBr (6 eq), (ii) NaH; f. (i) NaH, (ii) BnBr (5.5 eq); g. 1 M aq. NaOH, 1,4-dioxane, 70° C.; h. Ac₂O, Pyridine; i. TMSOTf, MS 4 Å, Toluene, 0° C.; j. TMSOTf, MS 4 Å, Toluene, −10° C. In FIG. 2D: a. MeONa, MeOH; b. Ba(OH)₂.8H₂O, H₂O, 90° C.; c. (Cl₃Ac)₂O, NaOMe, 0° C.; d. Ac₂O, Pyridine; e. H₂, cat. [Ir(COD){PCH₃(C₆H₅)₂}₂]⁺PF₆⁻, THF then I₂, H₂O; f. CCl₃CN, DBU, −5° C.; g. TMSOTf, MS 4 A, Toluene, −60° C.

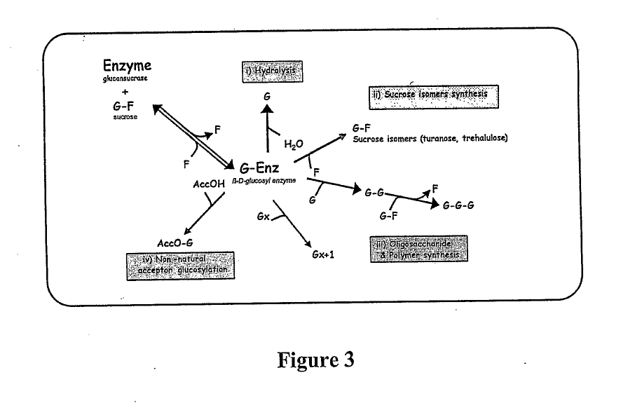

FIG. 3 shows the reaction catalyzed by glucansucrases. Glucansucrases follow a double displacement retaining mechanism, in which a β-glucosyl enzyme covalent intermediate is first formed from sucrose substrate. In a second step, the glucosyl moiety is transferred to an acceptor which depends on the conditions of reaction may be (i) water to give glucose (ii) fructose to form sucrose isomers (iii) glucose released from hydrolysis to form soluble oligosaccharides, or (iv) an exogeneous hydroxylated acceptor.

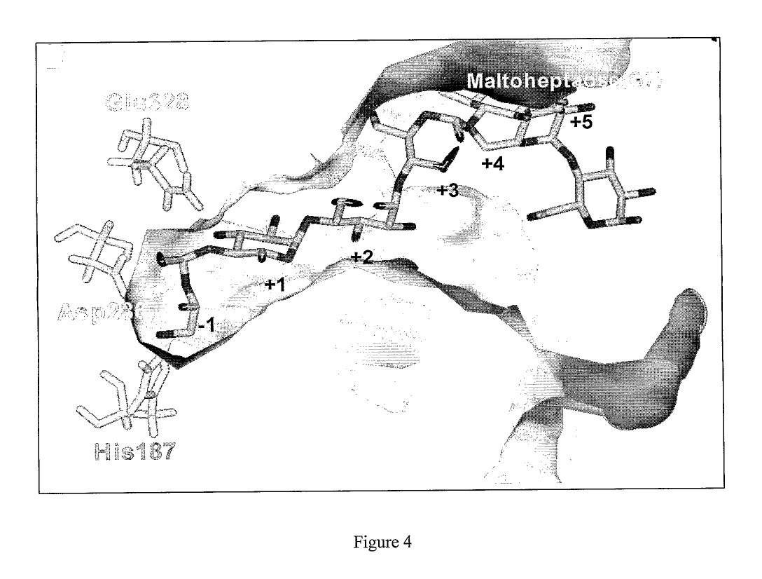

FIG. 4 shows the architecture of the active site in complex with maltoheptaose (G7).

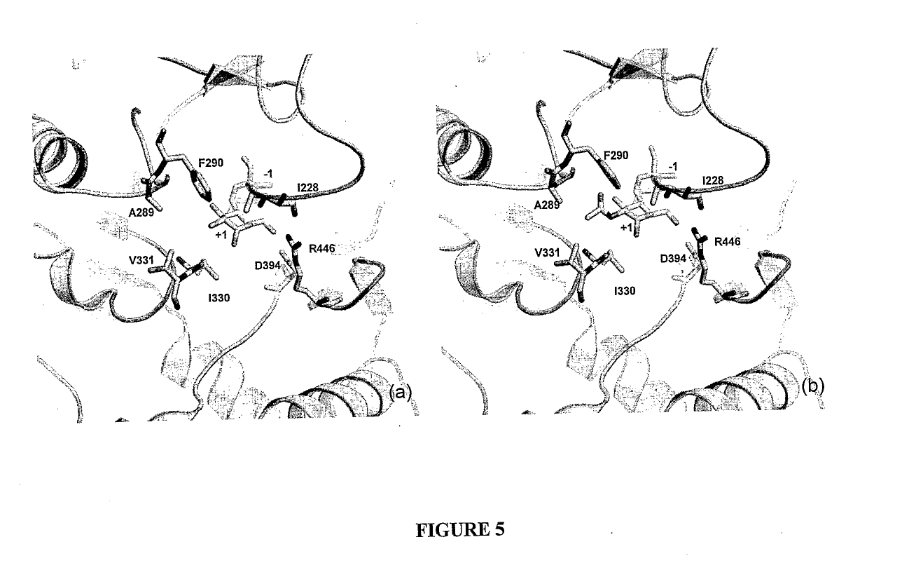

FIG. 5 shows the comparison of docking modes: (A) Maltose moiety from the crystallographic maltoheptaose (PDB: 1MW0) occupying binding subsites (−1) and (+1) of amylosucrase from Neisseria polysaccharea and (B) α-D-Glcp-(1→4)-D-GlcpNAc in the active site of AS. The seven amino acid residues (I228, A289, F290, I330, V331, D394 and R446) selected for mutagenesis are shown on the figures. Hydrogen atoms have been omitted on the figures for clarity purpose.

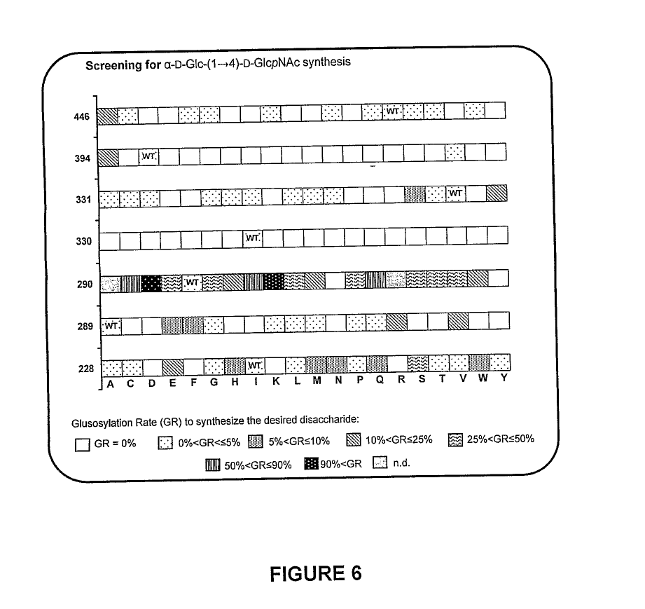

FIG. 6 shows the screening of the library for their ability to synthesize the desired disaccharide: α-D-Glcp-(1→4)-D-GlcpNAc. Rows indicate the 7 mutated positions and columns represent the 20 possible amino acid mutations including the wild type amylosucrase.

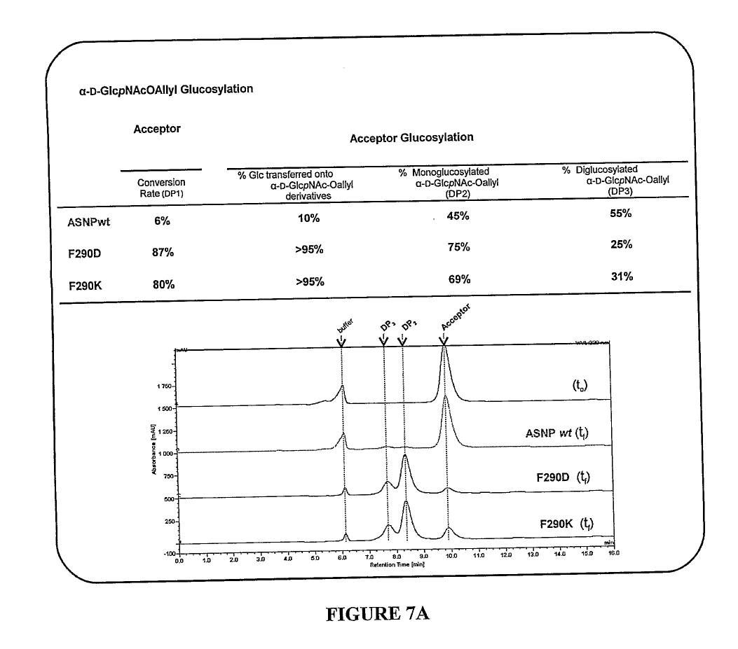

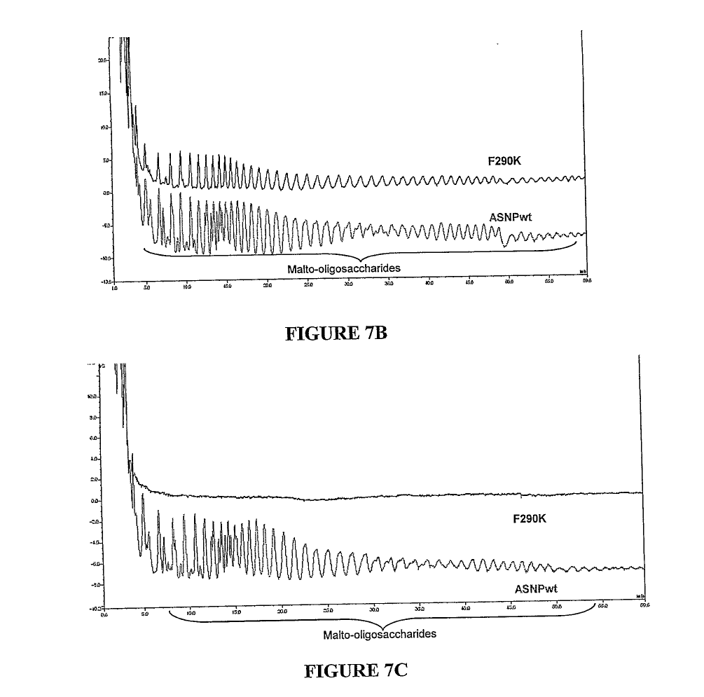

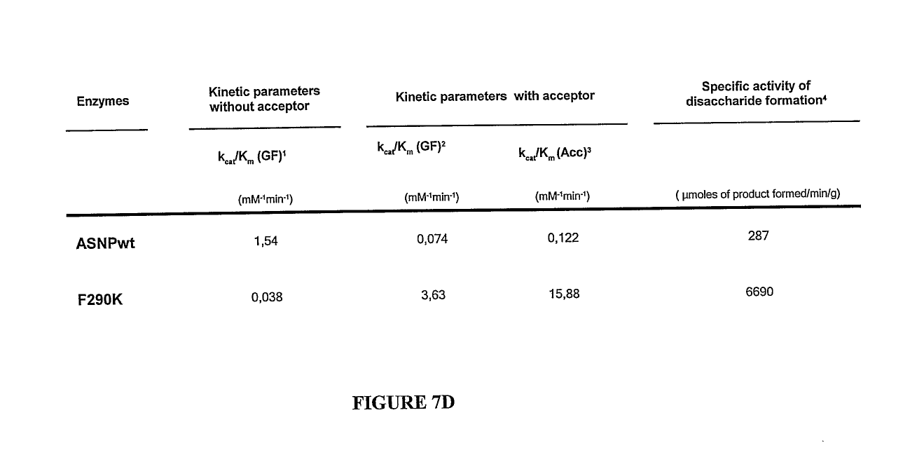

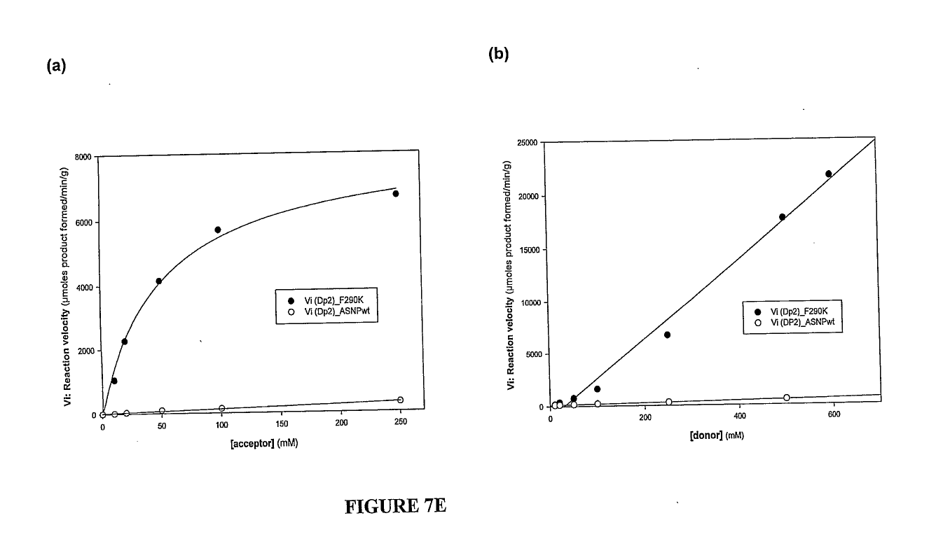

FIG. 7A shows transglucosylation of α-D-GlcpNAc-OAll using the most improved 1G5A variant F290K and F290D and corresponding HPLC chromatogramm (with UV_{λ=220 nm} detection) comparing F290K, F290D and ASNPwt. Initial reaction conditions: Sucrose=Acceptor=146 mM. At final time, sucrose was fully consumed. Conversion Rate=(Q(Acceptor)ₜ₀−Q(Acceptor)_{tf})/Q(Acceptor)ₜ₀ where Q(X)=Quantity of X in moles. % Glc transferred onto acceptor derivatives=[Q(Glucosyl units transferred onto acceptor derivatives)/Q(Glucosyl units transferable from initial sucrose)]×100. % Monoglucosylated acceptor=[Q(Monoglucosylated acceptor)/Q (acceptor derivatives)]×100. % Diglucosylated acceptor=[Q(Diglucosylated acceptor)/Q (acceptor derivatives)]×100. G=Glucose; F=Fructose; GF=Sucrose; DP₁=Acceptor; DP₂=monoglucosylated Acceptor; DP₃=diglucosylated Acceptor (to=initial time of the reaction−the medium contains only the donor and the acceptor, no products have been formed yet).

FIG. 7B shows the comparison of Dionex HPAEC product profiles obtained at the end of the reaction (t=24 h) with wild-type AS and the variant F290K using 146 mM sucrose.

FIG. 7C shows the comparison of Dionex HPAEC product profiles obtained at the end of the reaction (t=24 h) with wild-type AS and the variant F290K using 146 mM sucrose supplemented with 146 mM acceptor (α-D-GlcpNAc-OAll).

FIG. 7D shows the comparison of kinetic parameters between ASNPwt and the variant F290K.

FIG. 7E shows the determination of kinetic parameters for the variant F290K and ASNPwt catalyzed reactions: (a) varied acceptor (b) varied donor. FIG. 7E(a), varied acceptor: Allyl 2-/N/-acetyl-2-deoxy-α-D-glucopyranoside, constant donor: 250 mM. FIG. 7E(b), varied donor, constant acceptor: Allyl 2-/N/-acetyl-2-deoxy-α-D-glucopyranoside.

FIGS. 8.1 to 8.8 show the sequence alignment of 34 wild type glycoside hydrolases using the CLUSTALW program under default parameters.

FIG. 9 shows the alignment of the eleven different motifs found in 34 wild type glycoside hydrolases.

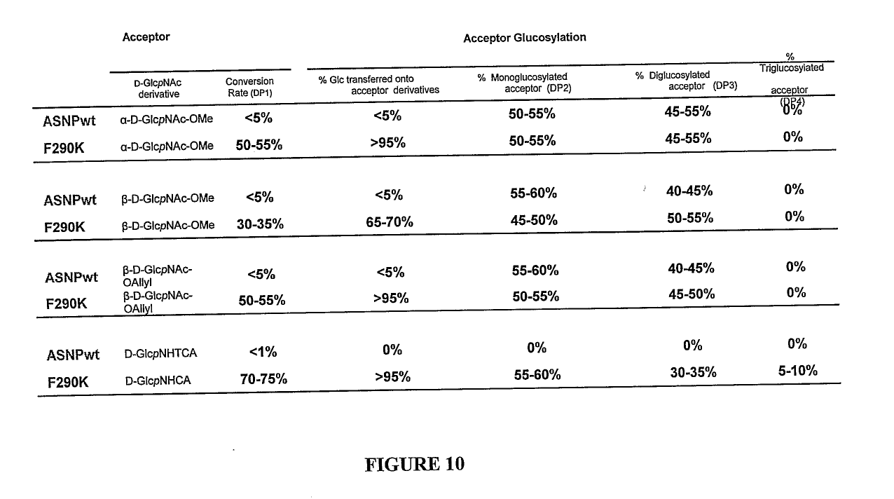

FIG. 10 shows transglucosylation rates of D-GlcpNAc derivatives using the most improved variant F290K and ASNPwt. Initial reaction conditions: Sucrose=Acceptor=146 mM. At final time, sucrose was fully consumed. Conversion Rate=(Q(Acceptor)ₜ₀−Q(Acceptor)_{tf})/Q(Acceptor)ₜ₀ where Q(X)=Quantity of X in moles. % Glc transferred onto acceptor derivatives=[Q(Glucosyl units transferred onto acceptor derivatives)/Q(Glucosyl units transferable from initial sucrose)]×100. % Monoglucosylated acceptor=[Q(Monoglucosylated acceptor)/Q(acceptor derivatives)]×100. % Diglucosylated acceptor=[Q(Diglucosylated acceptor)/Q (acceptor derivatives)]×100. % Triglucosylated acceptor=[Q(Triglucosylated acceptor)/Q(acceptor derivatives)]×100.

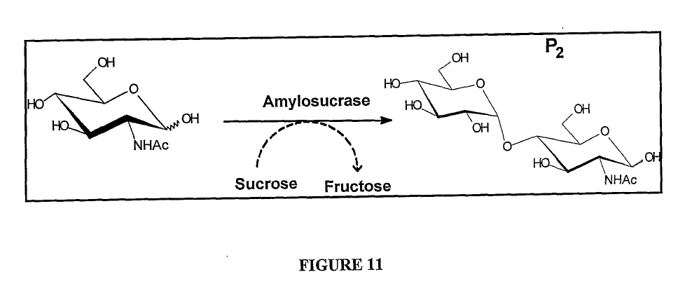

FIG. 11 shows the structure of disaccharide α-D-Glcp-(1→4)-D-GlcpNAc (P2) obtained by AS-mediated glucosylation of D-GlcpNAc.

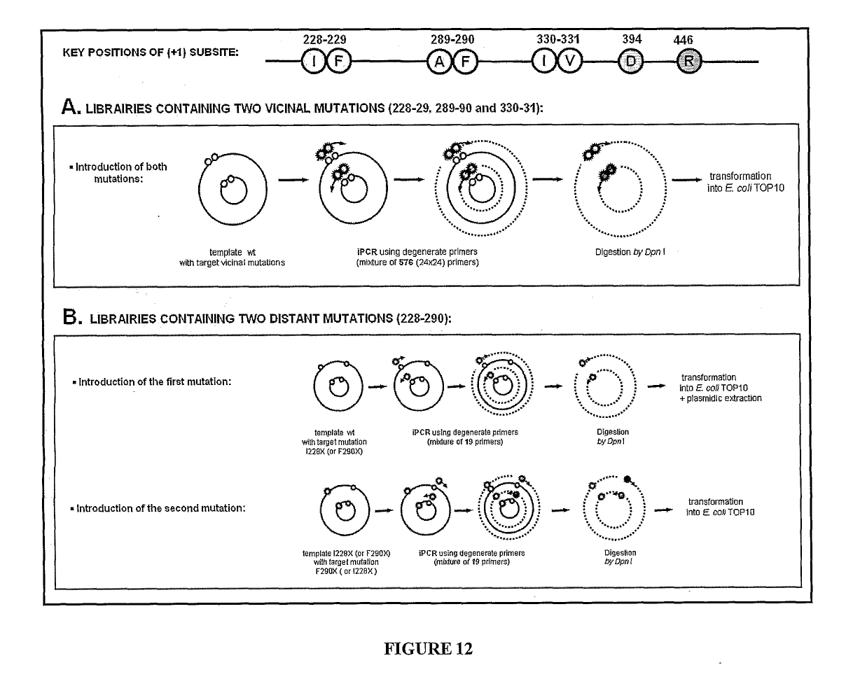

FIG. 12 shows the strategy adopted for the construction of the four double-mutant libraries; (A): libraries 1, 2 and 3; (B) library 4.

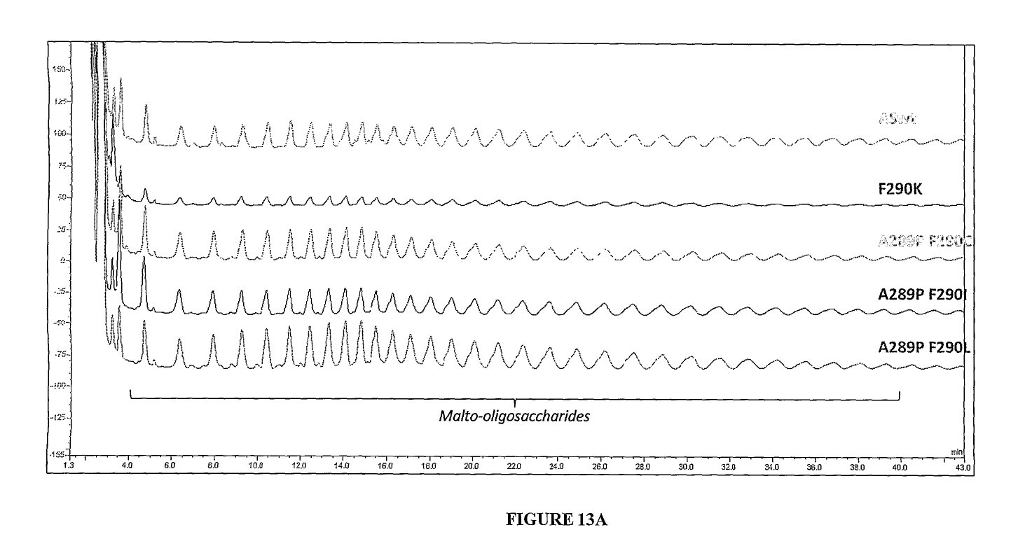

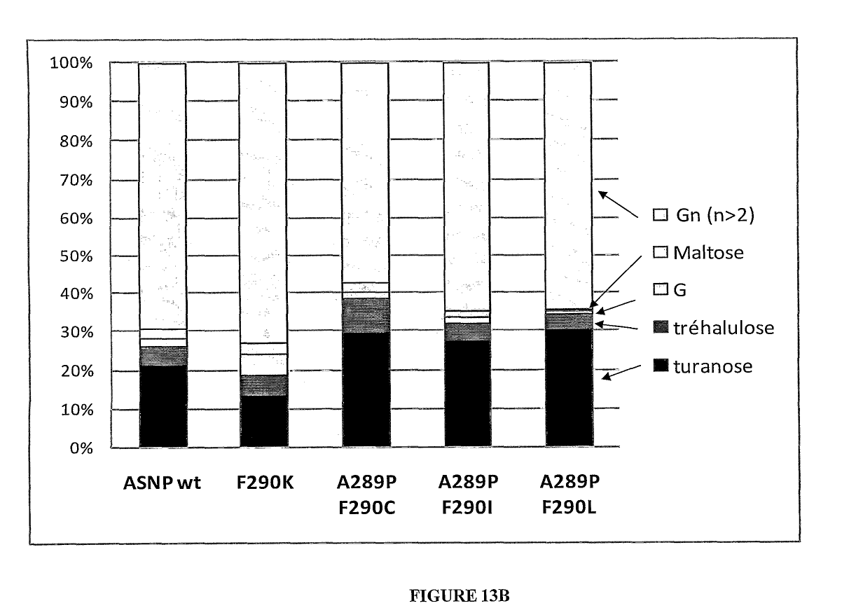

FIG. 13 shows the comparison of amylose synthesis by wtAS, variant F290K and double-mutants A289P-F290C, A289P-F290I, A289P-F290L from 250 mM sucrose. In FIG. 13A: superposition of the HPAEC-PAD profiles obtained at the end of the reaction (final time=24 h) with wtAS, variant F290K and double-mutants A289P-F290C, A289P-F290I, A289P-F290L. In FIG. 13B: yields of glucosyl units incorporated into the various products synthesized in the total reaction medium by wtAS, variant F290K and double-mutants A289P-F290C, A289P-F290I, A289P-F290L.

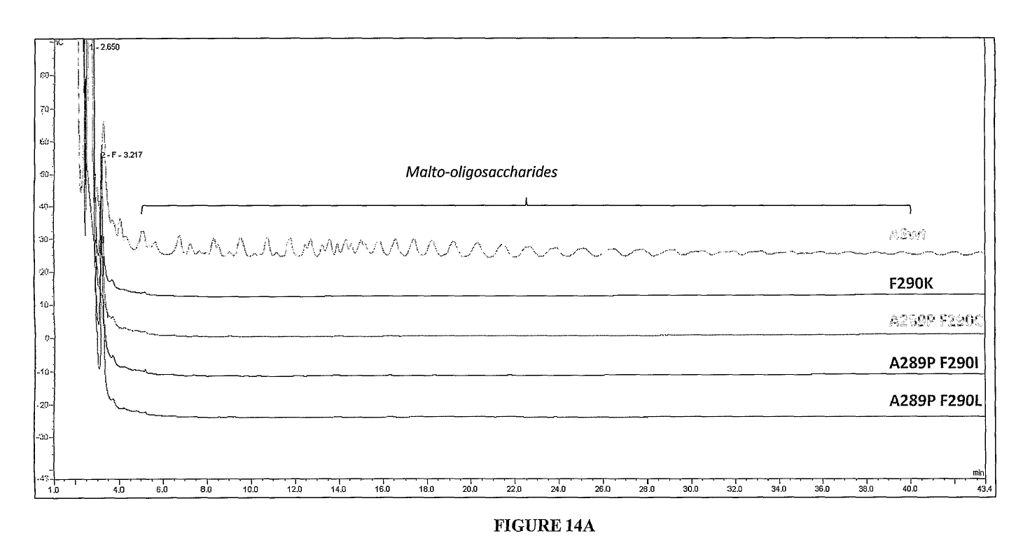

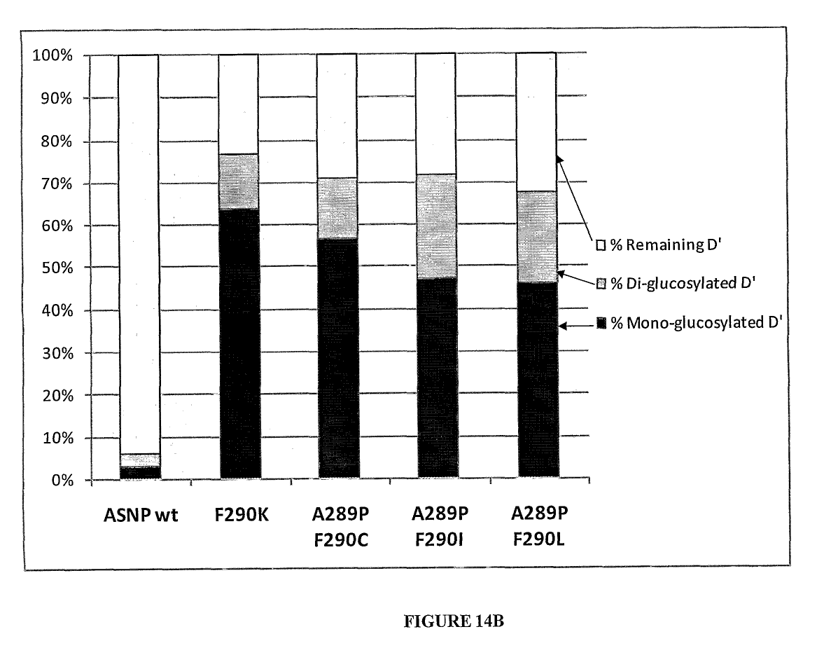

FIG. 14 shows the comparison of α-D-GlcpNAc-OAll (D′) transglucosylation with wtAS, variant F290K and double-mutants A289P-F290C, A289P-F290I, A289P-F290L from 250 mM sucrose supplemented with 250 mM α-D-GlcpNAc-OAll. In FIG. 14A: Superposition of the HPAEC-PAD profiles obtained at the end of the reaction (final time=24 h) with wtAS, variant F290K and double-mutants A289P-F290C, A289P-F290I, A289P-F290L. In FIG. 14B: Yields of mono- and di-glucosylated α-D-GlcpNAc-OAll obtained with wtAS, variant F290K and double-mutants A289P-F290C, A289P-F290I, A289P-F290L. At final time, >90% of sucrose was consumed.

### EXAMPLE 1

### Engineering Transglucosidase for the Synthesis the α-D-glucopyranosyl-(1→4)—N-acetyl-α/β-D-glucopyranosaminyl disaccharide

### 1) Materials and Methods

### Bacterial Strains, Plasmids and Chemicals

Plasmid pGST-AS, derived from the pGEX-6P-3 (GE Healthcare Biosciences) and containing the N. polysaccharea amylosucrase encoding gene (ref. 45) was used for the construction of the AS single mutant library.

E. coli JM109 was used as host for the plasmid library transformation, gene expression and large-scale production of the selected mutants.

Sucrose, N-acetyl-D-glucosamine and glycogen were purchased from Sigma-Aldrich (Saint-Louis, Mo., USA). Known allyl 2-acetamido-2-deoxy-α-D-glucopyranoside (α-D-GlcpNAc-OAll) (ref. 69), allyl 2-acetamido-2-deoxy-β-D-glucopyranoside (ref. 70) (β-D-GlcpNAc-OAll), methyl 2-acetamido-2-deoxy-α-D-glucopyranoside (ref. 71) (α-D-GlcpNAc-OMe), methyl 2-acetamido-2-deoxy-β-D-glucopyranoside (ref. 70) (β-D-GlcpNAc-OMe), and 2-deoxy-2-trichloroacetamido-β-D-glucopyranose (ref. 72) (D-GlcNHTCA) were synthesized chemically.

The reference disaccharides α-D-Glcp-(1→4)-D-GlcpNAc and Glcp-(1→6)-D-GlcpNAc were enzymatically synthesised and characterized (see Example 7).

Ampicillin, lysozyme and isopropyl β-D-thiogalactopyranoside (IPTG) were purchased from Euromedex (Souffelweyersheim, France), and DpnI restriction enzyme from New England Biolabs (Beverly, Mass., USA).

Oligonucleotides were synthesised by Eurogenetec (Liege, Belgium). DNA extraction (QIASpin) and purification (QIAQuick) columns were purchased from Qiagen (Chatsworth, Calif.).

Wild type glycoside hydrolase: amylosucrase (ASNPwt) 1G5A of sequence SEQ ID NO: 13.

### Selection of Mutation Position by Molecular Modelling

Starting models for the disaccharide and for AS: The disaccharide α-D-Glcp-(1→4)-D-GlcpNAc was constructed with the monosaccharide obtained from a database of carbohydrate three-dimensional structures. All molecular modelling calculations were performed using the SYBYL 7.3 software. The coordinates of amylosucrase were taken from the 2.0 Å resolution crystal structures of amylosucrase from N. polysaccharea in complex with sucrose (PDB: 1JGI) and maltoheptaose, a reaction product (PDB: 1MW0). All hydrogen atoms were added to the enzyme and their position optimized with the Tripos force field.

Systematic conformational search for the disaccharide: Both anomeric forms of the α-D-Glcp-(1→4)-D-GlcpNAc, were subjected to a systematic grid search study of the glycosidic linkage conformation. Starting from minimized disaccharides, a two-dimensional systematic conformational search was performed by rotating the two torsion angles defining the glycosidic linkages, Φ and Ψ. by 20° steps: Φ=O5′-C1′-O4-C4 and Ψ=C1′-O4-C4-C3 for α-D-Glcp-(1→4)-D-GlcpNAc. The MM3 force field implemented in SYBYL 7.3 software was used for this purpose together with the energy parameters appropriate for carbohydrates. Different maps were constructed with the dielectric constant set to 4.0 and 78.0 (to mimic gas phase and water environment, respectively). The geometries were optimized at each point of the grid with the driver option that keeps fixed the atoms defining the torsion angles. The solvent specific relaxed conformational maps obtained for all disaccharides were then used to locate the different energy minima that were subsequently fully relaxed.

Docking of disaccharide in the binding site of AS: The lowest energy conformations identified on the disaccharide potential energy maps were used as starting structures to be docked in the binding site of amylosucrase. This was performed by superimposing the glucosyl unit of the disaccharides at (−1) subsite onto the glucosyl unit of the crystallographic maltoheptaose. Each of these AS-disaccharide complexes was optimized by means of the appropriate energy parameters. The annealing method implemented in SYBYL 7.3 software was used to optimize the complexes. Two shells of amino acids were considered: a 12 Å shell centred on the binding site was taken into account for the energy calculations. A 6 Å shell region closest to the carbohydrate was defined as the hot region to be optimized. The position of all atoms included in this region was optimized using Powell's method.

### Construction of Mutant Library

Single mutagenesis, focused on +1 subsite amino acids retained from ligand docking, was carried out with the QuickChange Site-Directed Mutagenesis Kit (Stratagene, La Jolla, Calif.) according to the manufacturer's instructions, and using pGST-AS G537D as vector template. It was checked that this mutation had no impact on the native enzyme catalytic properties. The complementary primers listed below were used to obtain the single mutant library (Table II below). XXX codon indicates the bases which were used to obtain the replacement by the desired amino acids and are listed in Table III below.

TABLE II
Primers used to generate the 19 monomutants for
the positions 228, 289, 290, 330, 331,394 and 446.

| SEQ ID | Primer | Nucleotide |
|---|---|---|
| NO: | Name | Sequence |
| 14 | I228for | 5′-ACC CTG CGC GAA XXX TTC CCC GAC CAG CA-3′ |
| 15 | I228rev | 5′-TG CTG GTC GGG GAA XXX TTC GCG CAG GGT-3′ |
| 16 | A289for | 5′-T ATG GAT GCG GTT XXX TTT ATT TGG AAA CAA AT-3′ |
| 17 | A289rev | 5′-AT TTG TTT CCA AAT AAA XXX AAC CGC ATC CAT A-3′ |
| 18 | F290for | 5′-T ATG GAT GCG GTT GCC XXX ATT TGG AAA CAA AT-3′ |
| 19 | F290rev | 5′-AT TTG TTT CCA AAT XXX GGC AAC CGC ATC CAT A-3′ |
| 20 | I330for | 5′-TC AAA TCC GAA GCC XXX GTC CAC CCC GAC CAA GT-3′ |
| 21 | I330rev | 5′-AC TTG GTC GGG GTG GAC GAT XXX TTC GGA TTT GA-3′ |
| 22 | V331for | 5′-TC AAA TCC GAA GCC ATC XXX CAC CCC GAC CAA GT-3′ |
| 23 | V331rev | 5′-AC TTG GTC GGG GTG XXX GAT GGC TTC GGA TTT GA-3′ |
| 24 | D394for | 5′-TC CGC AGC CAC GAC XXX ATC GGC TGG ACG TTT-3′ |
| 25 | D394rev | 5′-AAA CGT CCA GCC GAT XXX GTC GTG GCT GCG GA-3′ |
| 26 | R446for | 5′-ACA GGC GAC TGC XXX GTC AGT GGT ACA-3′ |
| 27 | R446rev | 5′-TGT ACC ACT GAC XXX GCA GTC GCC TGT-3′ |

TABLE III
Sequence of XXX codon used to replace each selected amino acids
by the 19 other ones

| | | Ala | Cys | Asp | Glu | Phe | Gly | His | Ile | Lys | Leu | Met | Asn | Pro | Gln | Arg | Ser | Thr | Val | Trp | Tyr |
|---|---|---|---|---|---|---|---|---|---|---|---|---|---|---|---|---|---|---|---|---|---|
| 228 | for | GCC | TGC | GAC | GAG | TTC | GGC | CAC | wt | AAG | CTC | ATG | AAC | CCC | CAG | CGC | AGC | ACC | GTC | TGG | TAC |
| | rev | GGC | GCA | GTC | CTC | GAA | GCC | GTG | wt | CTT | GAG | CAT | GTT | GGG | CTG | GCG | GCT | GGT | GAC | CCA | GTA |
| 289 | for | wt | TGC | GAC | GAA | TTC | GGC | CAC | ATC | AAA | CTC | ATG | AAC | CCC | CAA | CGC | AGC | ACC | GTC | TGG | TAC |
| | rev | wt | GCA | GTC | TTC | GAA | GCC | GTG | GAT | TTT | GAG | CAT | GTT | GGG | TTG | GCG | GCT | GGT | GAC | CCA | GTA |
| 290 | for | GCT | TGT | GAT | GAA | wt | GGT | CAT | ATT | AAA | CTT | ATG | AAT | CCT | CAA | CGT | TCT | ACT | GTT | TGG | TAT |
| | rev | AGC | ACA | ATC | TTC | wt | ACC | ATG | AAT | TTT | AAG | CAT | ATT | AGG | TTG | ACG | AGA | AGT | AAC | CCA | ATA |
| 330 | for | GCC | TGC | GAC | GAA | TTC | GGC | CAC | wt | AAA | CTC | ATG | AAC | CCC | CAA | CGT | TCT | ACC | GTC | TGG | TAC |
| | rev | GGC | GCA | GTC | TTC | GAA | GCC | GTG | wt | TTT | GAG | CAT | GTT | GGG | TTG | ACG | AGA | GGT | GAC | CCA | GTA |
| 331 | for | GCC | TGC | GAC | GAA | TTC | GGC | CAT | ATC | AAA | CTC | ATG | AAC | CCC | CAA | CGC | TCC | ACC | wt | TGG | TAC |
| | rev | GGC | GCA | GTC | TTC | GAA | GCC | ATG | GAT | TTT | GAG | CAT | GTT | GGG | TTG | GCG | GGA | GGT | wt | CCA | GTA |
| 394 | for | GCC | TGC | wt | GAG | TTC | GGC | CAC | ATC | AAA | CTC | ATG | AAC | CCC | CAA | CGC | AGC | ACC | GTC | TGG | TAC |
| | rev | GGC | GCA | wt | CTC | GAA | GCC | GTG | GAT | TTT | GAG | CAT | GTT | GGG | TTG | GCG | GCT | GGT | GAC | CCA | GTA |
| 446 | for | GCT | TGT | GAT | GGT | TTT | GGT | CAT | ATT | AAA | CTT | ATG | AAT | CCT | CAA | wt | AGT | ACT | GTT | TGG | TAT |
| | rev | AGC | ACA | ATC | ACC | AAA | ACC | ATG | AAT | TTT | AAG | CAT | ATT | AGG | TTG | wt | ACT | AGT | AAC | CCA | ATA |

PCR amplification was carried out with Pfu DNA polymerase (2.5 U) for 16 cycles (95° C., 30s; 55° C., 30s; 72° C., 12 min). The DNA was digested with DpnI to eliminate methylated parental template and purified using Qiaquick spin column, following manufacturer's recommendations. E. coli JM109 was transformed with the plasmid and plated on LB agar supplemented with 100 μg/mL ampicillin. For each construction, two clones were isolated and their corresponding plasmids stored at −20° C. 17 mutants (I228A₁, I228V₁, I228Y1, A289D₁, F290D₁, F290K1, F290Q₁, I330A₁, I330D₁, I330E₁, I330F₁, I330T₁, I330W₁, V331A₁, V331S₁, D394V₁ and R446K₁) were sequenced on the entire gene and showed no other mutations by Millegen (Labège, France) or Cogenics (Meylan, France).

### Expression of Mutant Library

The protocol was established to enable the rapid identification of clones for which D-GlcpNAc glucosylation was improved. To obtain higher amounts of enzymes and facilitate detection of glucosylated compounds upon HPLC screening, mutants were produced in 96-DeepWell Format plates. Storage microplates containing monomutants were thawed and replicated to inoculate a starter culture in 96-well microplates containing, in each well, 150 μL LB medium supplemented with ampicillin (100 μg/mL). After growth for 24 h at 30° C. under agitation (200 rpm), plates were duplicated into 96-Deep Well plates containing, in each well, 1.1 mL LB medium supplemented with ampicillin (100 μg/mL) and IPTG (1 mM) to induce GST-AS expression. Cultures were then grown for 24 h at 30° C. under agitation (200 rpm). Plates were centrifuged (20 min, 3000 g, 4° C.) and the supernatant was removed. The cell pellet was resuspended in 200 μL of lysozyme (0.5 mg/mL), followed by freezing at −80° C. for 8 to 12 h. After thawing at room temperature, 100 μL of sucrose and 100 μL of acceptor (each at a final concentration of 73 mM) were added to each well. Enzymatic reaction was incubated at 30° C. during 24 h under agitation. The DeepWell plates were then centrifuged (20 min, 3000 g, 4° C.) and 300 μL of the supernatant was transferred to a filter micro-plate (PVDF 0.2 μm) to be clarified. Supernatant filtration was carried out by centrifugation of the filter micro-plate (5 min, 2000 g, 4° C.) into a novel microplate for screening.

### Development of the BBT Test in Liquid Medium

Mono-mutant library was cultured in 96-well microplates as previously described (ref. 81). After thawing at room temperature, 80 μL of the lysed cells were transferred into a new microtiter plate. Enzymatic reaction was carried out by adding 80 μL of sucrose to a final concentration of 146 mM followed by incubation at 30° C. for 24 h.

Bromothymol Blue (BBT) test: Medium acidification due to acid production consecutive to fructose release by action of active amylosucrase onto sucrose was determined by adding 1004 of the reaction mixture to 10 μL BBT solution (0.25% (g/v) dissolved in 1% ethanol) in a polystyrene plate. Absorbance was measured at 620 nm with the Sunrise™ microplate reader.

The fructose production was also followed by DNS (dinitrosalicylic) assay (ref. 57) for comparison. A volume of 100 μL of reaction mixture was added to 100 μL of dinitrosalicylic acid reagent in a propylene plate, incubating at 95° C. for 10 minutes. 100 μL of this mixture and 100 μL H₂O were transferred in a polystyrene microtiter plate. Absorbance at 540 nm was measured.

For Iodine staining assay (ref. 82), amylose formation was detected by adding 10 μL of iodine solution (100 mM KI, 6 mM I₂, 0.02 M HCl) to 50 μL of the reaction mixture. Absorbance was measured at 550 nm, the iodine forming a blue complex with the helical form of amylose.

### Development of the BBT Test at Colony Level

On day 1, libraries were transformed into electrocompetent E. coli TOP10 and plated on membranes (Durapore® membrane filters, 022 μm GV-Millipore, Ireland) which were previously soaked in physiological water and placed onto 22 cm square plates (Corning, USA) containing 200 mL solid LB agar, 1% glycerol and 100 μg/mL ampicillin.

On day 2, after overnight growth at 37° C. (˜16 h), each membrane was transferred on a 22 cm square plate containing inducing medium (200 mL solid LB agar +1 mM IPTG, 100 μg/mL ampicillin) supplemented with 50 g/L sucrose and stained in blue by adding 50 mM Tris-HCl pH=7.5 and 0.1 g/l pH indicator Bromothymol Blue (BBT, dissolved in 1% ethanol). The plates were incubated overnight (24 h) at 30° C.

On day 3, active clones (green and yellow) were isolated in microplates containing 200 μL LB medium, 12% glycerol and 100 μg/mL ampicillin. After 24 h of growth at 30° C., they were stored at −20° C. and −80° C. Inactive clones (blue) were scraped, cultivated and stored in the same conditions.

### Screening of Mutant Library

Efficiency of the glucosylation reaction was evaluated by HPLC analysis of the acceptor reaction product synthesized when using D-GlcpNAc as acceptor using a Dionex P 680 series pump, a Shodex RI 101 series refractometer, a Dionex UVD 340 UV/Vis detector and an autosampler HTC PAL. HPLC analyses were performed using a Biorad HPLC Carbohydrate Analysis column (HPX-87K column (300×7.8 mm)) maintained at 65° C., using ultra-pure water as eluent with a flow rate of 0.6 mL/min HPX-87K column was used to determine sucrose consumption by RI detection and concomitant α-D-Glcp-(1→4)-D-GlcpNAc formation by UV_{=220 nm} detection.

### Production and Purification of the Selected Variants: F290D and F290K

Production and purification of AS variants were performed as previously described (ref. 45). Since pure GST/AS fusion protein possesses the same function and the same efficiency as pure AS, enzymes were purified to the GST/AS fusion protein stage (96 kDa). The enzymes were desalted by size exclusion chromatography using P6DG columns (GE Healthcare Biosciences) at +4° C. and stored in elution buffer (50 mM Tris-HCl, pH 7.0, 150 mM NaCl) at −80° C. The protein content was determined by micro-Bradford method, using bovine serum albumin as standard (ref. 56).

### Biochemical Characterization of the Selected 1G5A Variants: F290D and F290K

All assays were performed at 30° C. in 50 mM Tris buffer, pH=7.0. For the acceptor reactions, F290D, F290K and ASNPwt (1G5A) were tested on α-D-GlcpNAc-OAll.

Standard activity determination. Specific activity of the purified enzymes was determined by measuring the initial rate of released fructose under standard conditions (146 mM sucrose). Fructose concentration was determined using the dinitrosalycilic acid (DNS) method (ref. 57). One unit of AS variant corresponds to the amount of enzyme that catalyses the production of 1 μmole fructose per minute in the assay conditions.

Comparison of products synthesized by wild-type and AS variants. Reactions were performed in the presence of 146 mM sucrose alone or supplemented with 146 mM acceptor. The purified wild-type or mutated GST/AS were employed at 0.5 U/mL. The reactions were stopped by heating at 95° C. for 5 min. The final mixture was centrifuged at 18 000 g for 10 min, filtered on 0.22 μm membrane and analyzed by HPLC, as previously described.

Different carbohydrate analyses were performed to compare the product profiles synthesized by ASNPwt and AS variant (F290K):

Soluble and insoluble oligosaccharides produced during the reaction were identified by HPAEC using a Dionex Carbo-Pack PA100 column at 30° C. Before analysis, the insoluble fraction was solubilized in KOH at a final total sugar concentration of 10 g/kg. Mobile phase (150 mM NaOH) was set at 1 mL/min flow rate with a sodium acetate gradient (going from 6 to 500 mM within 120 min). Detection was performed using a Dionex ED40 module with a gold working electrode and an Ag/AgCl pH reference. Note that α-D-GlcpNAc-OAll (acceptor) and its derivatives are not oxidable products and thus are not detectable by HPAEC. Sucrose, glucose, fructose, α-D-GlcpNAc-OAll (acceptor) and its derivatives (glucosylation products) were quantified by HPLC, as previously described

Kinetics Studies. (k_{cat},K_{M})

Enzyme assays were carried out in a total volume of 2 mL containing pure enzyme (0.115 mg and 0.106 mg when using ASNPwt and F290K, respectively). Catalytic efficiency (Eff=k_{cat}/K_{M}) of ASNPwt and F290K variant was determined with both sucrose (D=Donor) and α-D-GlcpNAc-OAll (A=Acceptor) as variable substrates. For the determination of the catalytic efficiency for D, A was held constant at 250 mM and D was varied between 0 and 600 mM. For the determination of the catalytic efficiency for A, D was held constant at 250 mM and A was varied between 0 and 250 mM. Experiments were performed until A and D solubility limits were reached. For each experiment, the reaction velocity corresponding to the acceptor glucosylation was determined by the formation of α-D-Glcp-(1→4)-α-D-GlcpNAc-OAll (called Vi(Dp2)), corresponding to the kinetics of the reaction of interest.

Initial velocities were fitted to the Michaelis-Menten equation using Sigma-Plot. As saturation was not achieved with the mutant, efficiency was calculated by linear regression analysis of the velocity versus substrate concentration plot. Aliquots (200 μL) were removed between 0 and 60 min (at which time product formation was still linear with respect to time), heated at 95° C. for 5 min and centrifuged at 18 000 g for 5 min. The final mixtures were filtered on a 0.22 um membrane and analyzed using HPLC material previously described. HPLC analyses were performed using a Biorad HPLC Carbohydrate Analysis column (HPX-87K column (300×7.8 mm)) maintained at 65° C., using ultra-pure water as eluent with a flow rate of 0.6 mL/min to determine the released fructose by RI detection and to detect the formation of α-D-Glcp-(1→4)-α-D-GlcpNAc-OAll by UV_{=220 nm} detection.

### Preparative Synthesis of the Acceptor Reaction Product

In order to characterize the products of D-GlcpNAc glucosylation, a synthesis was carried out at the preparative scale. 10 ml mixture containing 146 mM sucrose, 146 mM D-GlcpNAc and 1.5 U/ml of purified F290K AS mutant were incubated at 30° C. for 24 h. Then, the reaction mixture was centrifuged (4800 rpm, 20 min, 4° C.) to remove proteins and filtered on 0.22 μm membrane.

In the same way, oligosaccharides produced from α-D-GlcpNAc-OAll glucosylation were produced in a 100 mL mixture reaction (146 mM sucrose+146 mM α-D-GlcpNAc-OAll), using 4 U/mL of non-purified F290K extract (sonication supernatant).

### Purification of the Oligosaccharides

Concerning D-GlcpNAc glucosylation, monoglucosylated product was separated by preparative chromatography on C18 reverse-phase chromatography column (Bischoff Chromatography). Ultra pure water was used as eluent at a constant flow rate of 50 mL/min. Glucosyl detection was carried out with a refractometer (Bischoff) and each peak was collected separately, concentrated and reinjected into the analytical HPLC system described above, to verify the compound's purity.

### Structural Analysis of the Acceptor Reaction Products

The structure of Dp2, which was synthesized using sucrose as donor and D-GlcpNAc as acceptor with AS mutant F290K was analyzed by HRMS and NMR. It corresponds to α-D-glucopyranosyl-(1→4)—N-acetyl-D-glucosamine and was found identical to Dp2 fowled by ASNPwt.

HRMS (FAB): Anal. Calcd for C₁₄H₂₅NO₁₁Na: 406.1325 [MNa⁺], Found: 406.1356; ¹H and ¹³C listed in the Table IV below.

TABLE IV
¹H and ¹³C chemical shifts (ppm, D₂O) and ³J coupling constants (Hz) of P2
resulting from F290K-mediated glucosylation of D-GlcpNAc

| | | δ | Carbohydrate Ring | | | | | | N-Acetyl | |
|---|---|---|---|---|---|---|---|---|---|---|
| Compounds | Residue | (ppm) | 1 | 2 | 3 | 4 | 5 | 6 | C | CH₃ |
| P2 | α-D-Glcp | ¹H | 5.39 | 3.56 | 3.66 | 3.40 | 3.70 | 3.82 | | |
| (β anomer) | | ¹³C | 99.87 | 72.06 | 73.27 | 69.78 | 73.14 | 60.97 | | |
| | β-D- | ¹H | 4.70 | 3.68 | 3.79 | 3.69 | 3.56 | 3.82 | | 2.02 |
| | GlcpNAc | | (J_{1,2}: 8.0 Hz) | | | | | | | |
| | | ¹³C | 95.25 | 57.09 | 74.79 | 77.22 | 75.06 | 61.00 | 175.22 | 22.62 |
| P2 | α-D-Glcp | ¹H | 5.40 | 3.56 | 3.66 | 3.40 | 3.70 | 3.82 | | |
| (α anomer) | | ¹³C | 100.15 | 72.17 | 73.30 | 69.78 | 73.14 | 60.97 | | |
| | α-D- | ¹H | 5.18 | 3.89 | 4.00 | 3.66 | 3.94 | 3.82 | | 2.02 |
| | GlcpNAc | | (J_{1,2}: 3.6 Hz) | | | | | | | |
| | | ¹³C | 91.13 | 54.36 | 71.62 | 77.94 | 70.57 | 61.00 | 174.98 | 22.34 |

### 2) Results

### Choice of Acceptor Substrates

The choice of a suitable precursor to residue D (FIG. 1) to be used as acceptors in the enzymatic steps to S. flexneri serotype 1a and 1b O-antigen was crucial. Three major features had to be taken into account: (i) light protecting pattern compatible with the limited ability of selected glucansucrases to modulate their acceptor binding site, (ii) easy synthetic access, and (iii) the possible conversion of the glucosylation product into a protected disaccharide building block known to be compatible with additional chemical elongation. The selection of an appropriate precursor to residue D (D′) was made according to the same criteria. Allyl 2-N-acetyl-2-deoxy-α-D-glucopyranoside (XX₁) was selected based on the assumption that the 3_{D′}-OH group would be easily differentiated at the disaccharide level providing that a 2,3-oxazolidinone moiety could be introduced following N-deacetylation. Regioselective differentiation of the 3_{D′}-OH is indeed a pre-requirement to any specific chain elongation at this position as required in the synthesis of S. flexneri 1a and 1b oligosaccharides.

Thus, isolation of ED′ (XX₃) was best performed following rough chromatography of the crude enzymatic glucosylation mixture issued from D′ (XX₁), peracetylation into pure XX₄, then transesterification of intermediate XX₄ (30% from XX₁) (Scheme 2A). To access S. flexneri oligosaccharides bearing the ED branching pattern, the product of XX₁ enzymatic glucosylation, disaccharide XX₃, had to be turned into a donor allowing chain elongation at the reducing end (XX₁₆) (Scheme 2D) and/or an acceptor allowing chain extension at position 3_{D′}, XX₁₀ (Scheme 2C). Therefore, selective N-trichloroacetylation and per-O-acetylation of XX₅, resulting from extensive deacetylation of XX₄, gave XX₁₄ (58%), which was converted, via hemiacetal XX₁₅, to trichloroacetimidate XX₁₆ (26%) bearing a participating group at position 2_{D} as required (Scheme 2D). Alternatively, XX₄ was saponified into XX₅ (83%) (Scheme 2C), which was turned into the benzylated 2_{D},3_{D}-oxazolidinone XX₈ via XX₆ (34%). Oxazolidinone clivage and subsequent N-acetylation gave acceptor XX₁₀ (80%) bearing a free hydroxyl group at position 3 and a participating group at position 2. Both disaccharide donor XX₁₆ and disaccharide acceptor XX₁₀ were converted to trisaccharides by reaction with a rhamnopyranoside acceptor or a rhamnopyranosyl donor.

### Development of a Colorimetric Assay for Detecting Sucrose-Utilizing Variants

E. coli strains derived from E. coli K12 are unable to use sucrose as substrate. This property was used to develop a colorimetric screening test that allows isolation of recombinant E. coli clones on solid medium and the determination of the ratio of active clones present in a library of variants. The principle is based on the fact that active amylosucrase produced by recombinant E. coli will cleave sucrose and release fructose. This latter can enter the glycolytic pathway to produce acids and induce pH changes that cab be easily detected by using an appropriate pH colorimetric indicator. In the absence of an active glucansucrase, no acid production occurs and, thus, no change in the pH indicator color is observed.

To set up this assay and validate the new pH-based screen of active amylosucrase-producing clones, the method was applied to the library of 133 mono mutants constructed. After growth in microtiter plates, the cells were broken and incubated with sucrose for 24 hours, before adding the Bromothymol Blue (BBT). In parallel, a duplicate plate was analyzed using the dinitrosalicylic assay (DNS) (ref. 57) which enables the detection of the reducing power released from sucrose cleavage. The color of some wells changed from blue (pH=7.5) to green (pH˜6-7) or yellow (pH<6). This indicates that for some clones, an acidification of the medium occurred, thus revealing their ability to cleave sucrose. The slight change in color observed further shows that some clones produced either low levels of active AS or AS with a lower activity. 54% of active clones were detected in this library. By comparison, only 31% of the recombinant clones were detected as active with the DNS assay. Notably, all the clones detected with the DNS assay were also detected using the new colorimetric assay

### Screening Using BBT Colorimetric Test at Colony Level

BBT test was then used to develop a simple and highly sensitive staining method to detect clones producing active amylosucrase on solid medium. BBT pH indicator was directly introduced into solid LBₐₘₚ medium supplemented with sucrose, IPTG and Tris-Buffer at pH=7.5 to maintain medium staining (blue color). The BBT concentration was first optimized from 0.05 g/L to 0.5 g/L. It was found that 0.1 g/L of BBT offered the best contrast between blue and yellow colonies after incubation at 30° C. during 48 h. This protocol was first applied to freshly transformed cells and it was observed that transformation yield was much lower than that observed in usual conditions. In addition, colony development was also affected. Therefore, in order to ensure a good revival of the colonies, bacteria were first plated on a square plate containing LBₐₘₚ agar medium supplemented with 1% glycerol on which a hydrophilic membrane (polyvinylidene fluoride) had been overlaid. The plates were then incubated at 37° C. overnight to ensure colony development. The membrane was then transferred onto a second square plate containing LBₐₘₚ agar medium supplemented with sucrose, IPTG (inductor), Tris-Buffer at pH=7.5 and BBT. After 24 h incubation at 30° C., the colonies were easily differentiated, picked and cultured without any loss of viability. This procedure was subsequently applied to the library screening.

### Screening for Native Transglucosidases Able to Synthesize the Starting Building Block

Selected α-retaining transglucosidases are glucansucrases found in families 13 and 70 of glycoside-hydrolases (ref. 51). They catalyze the synthesis of α-glucan polymers by successive transfers of α-D-glucopyranosyl units from sucrose without any mediation of sugar nucleotides. Using the high energy of the sucrose bond to catalyze condensation reaction, they stand among the most efficient transglucosidases in the glycoside-hydrolase family. Depending on regiospecificity of the enzyme, distinct types of glucosidic linkage are found in the polymer formed. Notably, polymerization reaction can be redirected toward the glucosylation of exogenous acceptors, when the latter are well recognized (FIG. 3). In addition, glucansucrases generally possess a broad acceptor spectrum, what indicates a certain plasticity of the acceptor recognition at the acceptor binding site. However, none of them had yet been tested for the glucosylation of the starting acceptor of interest. Glucosylation of D-GlcpNAc was thus attempted with four recombinant glucansucrases, which were selected for their very distinct specificities. For this first screening it has been preferred to start with commercially available D-GlcpNAc instead of the allyl-derivative, assuming that the modification with an allyl group at the anomeric position would not significantly affect the acceptor recognition as preliminary studies performed on D-Glcp and α-D-Glcp-OAlkyl derivatives with glucansucrases previously showed negligible differences. Enzymes specific for α-1,6 and α-1,3, α-1,2 or α-1,4 glucosidic bond formation were tested in the presence of the target acceptor. None of them was able to glucosylate this acceptor with the requisite regiospecificity and good yields.

Consequently, to overcome the limited substrate recognition by the enzymes, it was opted for the engineering of novel transglucosidases with altered regio and stereospecificities. The combination of combinatorial and rational enzyme engineering in the form of focused small size libraries was used. Further, the three-dimensional structure of AS (ref. 52) was available in complex with either the substrate or the natural reaction product.

### Engineering of Amylosucrases Able to Glucosylate the Target Building Block

To modify enzyme specificity, an approach based on site-directed evolution targeted at the binding pocket was followed. The catalytic site pocket is defined by the subsites (−1) and (+1) according to the nomenclature earlier described for glycoside hydrolases (FIG. 4). The subsite (−1) is responsible for the specificity towards sucrose and is occupied by the glucosyl unit which will be transferred whereas the subsite (+1) ensures a correct positioning of the acceptor and is also responsible for specificity of synthesis of the (α-1→4) glucan linkage (ref. 46).

It was combined both the rational selection of mutation targets at the acceptor binding site (noted +1 subsite in FIG. 4) and for each of the identified positions, a systematic modification of the residue by all 19 remaining possible amino acids. As (+1) subsite is responsible for the enzyme specificity toward acceptors, the approach consisted in (i) mapping the binding site residues important for functional plasticity and (ii) identifying the most promising positions to be modified to favour acceptor recognition. Starting from the crystallographic structure of AS in complex with sucrose (PDB: 1JGI) (ref 54) and maltoheptaose (PDB: 1MW0) (ref 55), the target disaccharide α-D-Glcp-(1→4)-D-GlcpNAc was docked in the AS active site (FIG. 5).

Structural Analysis of α-D-Glcp-(1→4)-D-GlcpNAc [ED]: AS complex

The desired disaccharide α-D-Glcp-(1→4)-D-GlcpNAc was docked into the AS active site using the crystallographic maltose glucosyl units (i.e. α-D-Glcp-(1→4)-D-Glcp: native product) bound at (−1) and (+1) subsites (PDB: 1MW0) as a template for the starting location. As amylosucrase synthesizes naturally an α-(1-4) osidic linkage, the docking mode adopted by α-D-Glcp-(1→4)-D-GlcpNAc (target product) was close to the one observed for maltose glucosyl units at subsites (−1) and (+1) by crystallography. The unique difference between both disaccharides is the N-acetyl group at the C2 carbone of the D-GlcpNAc unit that substitutes the hydroxyl group carried by the D-Glcp in maltose. Modeling results indicated that to accommodate the D-GlcpNAc moiety at the (+1) subsite, several hydrophobic residues (Ala289, Phe290, Ile330 and Val331) surrounding the N-Acetyl group had to move away to provide enough fitting space. Monomutants for position Ile228, Asp394 and Arg446 have been also considered in the screen.

Out of the 18 residues identified as surrounding the (+1) subsite, 7 positions that were presumed to be not critical for sucrose binding but beneficial for target acceptor glucosylation have been selected for mutagenesis: Ile228, Ala289, Phe290, Ile330, Val331, Asp394 and Arg446. It was systematically mutated the selected amino acids by the 19 other possible residues to create a small size library focused on +1 subsite.

### Detection of Mutants Able to Glucosylate the Target Acceptors

For each of the 7 selected positions, 19 single mutants were generated (corresponding to each possible amino acid change). Site-directed mutagenesis has been preferred to saturation mutagenesis, as each mono mutant generated is directly identified and can be easily isolated and characterized. A first library of 133 monomutants (7×19) was thus obtained and stored in cryotubes and 96-wells microplates. The mutants were tested for the glucosylation of the target acceptor D-GlcpNAc in microtiter format experiments. HPLC screening was performed to identify those able to form the desired disaccharide. Both sucrose consumption and disaccharide formation were determined in order to calculate the glucosylation rate defined as the molar ratio of monoglucosylated acceptor versus/sucrose consumed.

D-GlcpNAc was poorly recognized by the wild type AS (glucosylation rate=2%). The remodelling of the +1 subsite led to performant results. Indeed, 17 mutants catalyzed the formation of the α-D-Glcp-(1→4)-D-GlcpNAc with a glucosylation rate comprised between 10 and 50% and 5 mutants with a glucosylation rate higher than 50% (FIG. 6). Position 290 is clearly a key position to improve the formation of α-D-Glcp-(1→4)-D-GlcpNAc. Of the 19 mutants, five synthesize the desired product with the correct regiospecificity and a glucosylation rate higher than 50%. Notably, F290D and F290K yielded the desired disaccharide with glucosylation rate of more than 90%, which represent a 45 fold increase compared to the wild type. Mutations at positions 228, 289, 331 and 446 also led to the improvement of the α-D-Glcp-(1→4)-D-GlcpNAc synthesis.

In overall, two mutants of interest for the chemo-enzymatic pathway were retained and further characterized F290D and F290K which are specific for the production of α-D-Glcp-(1-4)-D-GlcpNAc.

### Characterization of F290K and F290D 1G5A Mutants

F290D and F290K were produced in a larger amount and purified to homogeneity for further characterization. Glucosylation reactions were performed using α-D-GlcpNAc-OAll as acceptor with F290D and F290K. The distribution of the acceptor reaction products is shown in FIG. 7A. Regarding the variant F290K and F290D, both are highly specific for the glucosylation of α-D-GlcpNAc-OAll. F290K and F290D yielded comparable amount of α-D-Glcp-(1→4)-α-D-GlcpNAc-OAll and are 10 times more efficient for α-D-GlcpNAc-OAll glucosylation; 20 times more efficient to form α-D-Glcp-(1→4)-D-GlcpNAc-OAll. The best catalytic efficiency is however obtained with the F290K mutant, which was thus selected to carry out the production of α-D-Glcp-(1→4)-D-GlcpNAc-OAll.

Regarding the characterization of the products formed by F290K mutant, it was observed that addition of acceptor totally suppressed maltooligosaccharide formation (FIGS. 7B and 7C). Note that in the absence of acceptor, this mutant mainly catalyzed hydrolysis reaction but was still able to synthesize longer maltooligosaccharides (up to DP 20). Our assumption regarding the influence of the allyl aglycone in α-D-GlcpNAc-OAll on the glucosylation rate and regiospecificity of glucosaminyl acceptors was thus valid.

Kinetic measurements showed that F290K could not be saturated with sucrose, indicating thus a poor affinity for sucrose. k_{cat}/Km values were then determined by linear regression both in the presence and in the absence of acceptor (FIG. 7D). In the presence of sucrose alone, k_{cat}/Km values decreased by 40 fold for F290K mutant. In the presence of the acceptor, it was observed for F290K mutant a formidable increase of catalytic efficiency towards both sucrose and the acceptor (FIGS. 7D and 7E). Such an improvement can be attributed to the high specificity of the mutant F290K for the α-D-Glcp-(1→4)-D-GlcpNAc-OAll formation. The adaptation of the acceptor binding site to the target acceptor enhanced the rate of the de-glucosylation step, which is no more the limiting step of the reaction. A remarkable 130 fold increase of the efficiency of the mutant compared to ASNPwt was observed and the expected regioselectivity was achieved.

### EXAMPLE 2

### Enzymatic Glucosylation of D-GlcpNAc Derivatives

The mutant F290K and the ASNPwt were tested on other D-GlcNAc derivatives (α-D-GlcpNAc-OMe, β-D-GlcpNAc-OMe, β-D-GlcpNAc-Oallyl and D-GlcNHTCA) used as potential acceptors. The enzymatic reaction was carried out using sucrose as donor and D-GlcNAc derivative as acceptor in a 1:1 ratio (146 mM)

The distribution of the acceptor reaction products is shown in FIG. 10. Comparing to ASNPwt, the variant F290K is highly specific for the glucosylation of all the other D-GlcNAc derivatives. (>95% for α-D-GlcpNAc-OMe, β-D-GlcpNAc-OAll and D-GlcNHTCA and 65-70% for β-D-GlcpNAc-OMe).

Efficiency of the glucosylation reaction was evaluated by HPLC analysis of the acceptor reaction product synthesized a Biorad HPLC Carbohydrate Analysis column (HPX-87K column (300×7.8 mm))

Percentages of glucosylation rates were estimated according to peak areas as no reference was available.

### EXAMPLE 3

### Chemo-Enzymatic Synthesis of Potential Building Blocks to Oligosaccharide Fragments of S. flexneri 1b and/or 1a O-antigens

General Methods. All moisture sensitive reactions were carried out under an atmosphere of argon in oven-dried glassware. Anhydrous solvents sold on molecular sieves were used as such. 4 Å powder molecular sieves was kept under vacuum and activated before use by heating at 250° C. under vacuum. TLC were performed on precoated slides of Silica Gel 60 F₂₅₄ (Merck). Detection was effected with UV light, and/or by charring in 5% sulfuric acid in ethanol. Preparative chromatography was performed by elution from columns of Silica Gel 60 (particle size 0.040-0.063 mm) Products were routinely analyzed by ¹H and ¹³C NMR spectroscopy and by mass spectrometry (MS). NMR spectra were recorded at 25° C. (400 MHz for ¹H, 100 MHz for ¹³C). Proton-signal assignments were made by first-order analysis of the spectra, as well as analysis of 2D ¹H—¹H correlation maps (COSY). The ¹³C NMR assignments were supported by 2D ¹³C—¹H correlations maps (HSQC and HMBC). Signal assignments marked with a * are interchangeable assignments.

Allyl 2-acetamido-2-deoxy-α-D-glucopyranoside (XX₁) (ref. 69). A mixture of 2-acetamido-2-deoxy-D-glucose (50 g, 226 mmol), allyl alcohol (400 mL), and BF₃.OEt₂ (7.5 mL, 61 mmol) was heated at 90° C. After 24 h, the reaction mixture was concentrated under reduced pressure, and the resulty gummy residue was purified by column chromatography (9:1 CH₂Cl₂—MeOH) to obtain compound XX₁ as a white solid (49.6 g, 84%). Compound XX₁ had Rf=0.5 (85:15 CH₂Cl₂—MeOH); ¹H NMR (D20, 400 MHz) δ (ppm): 5.87 (m, 1H, CH═), 5.25 (m, 1H, ═CH₂), 5.17 (m, 1H, ═CH₂), 4.83 (d, 1H, J₁₋₂=3.6 Hz, H1), 4.13 (m, 1H, OCH₂), 3.94 (m, 1H, OCH₂), 3.82 (dd, 1H, J₂₋₃=10.7 Hz, H2), 3.78 (dd, 1H, J₅₋₆ₐ=2.2 Hz, J_{6a-6b}=12.0 Hz, H6a), 3.69 (dd, 1H, H6b), 3.67 (dd, 1H, J₃₋₄=9.7 Hz, H3), 3.63 (ddd, 1H, J₄₋₅=9.9 Hz, J_{5-6b}=5.6 Hz, H5), 3.39 (t, 1H, H4), 1.94 (s, 3H, COCH₃); ¹³C NMR (D₂O, 100 MHz) δ (ppm): 174.5 (C═O), 133.7 (CH═), 117.9 (═CH₂), 96.2 (C1), 72.0 (C5), 71.1 (C3), 70.1 (C4), 68.5 (OCH₂), 60.6 (C6), 53.7 (C2), 21.9 (COCH₃). FIRMS (ESI⁺) of C₁₁H₁₉NO₆Na ([M+Na]⁺, 284.1110) m/z 284.1112.

Allyl α-D-glucopyranosyl-(1→4)-α-D-glucopyranosyl-(1→4)-2-acetamido-2-deoxy-α-D-glucopyranoside (XX₂). The crude mixture issued from the enzymatic glucosylation of XX₁ (3.80 g, 14.54 mmol) was purified by silica gel column chromatography (9:1 CH₃CN—H₂O) to give a mixture of allyl α-D-glucopyranosyl-(1→4)-2-acetamido-2-deoxy-α-D-glucopyranoside (XX₃) and fructose (4.16 g) and of the pure trisaccharide XX₂ (2.53 g, 4.32 mmol) resulting from the enzyme-mediated transfer of glucose onto disaccharide XX₃. Trisaccharide XX₂ had Rf=0.3 (9:1 CH₃CN—H₂O). ¹H NMR (D₂O, 400 MHz) δ (ppm): 5.91 (m, 1H, CH═), 5.35-5.32 (m, 2H, H1_{E}, H1_{E′}), 5.29 (m, 1H, ═CH₂), 5.20 (m, 1H, ═CH₂), 4.86 (d, 1H, J₁₋₂=3.5 Hz, H1_{D}), 4.15 (m, 1H, OCH₂), 4.01-3.93 (m, 2H, H3_{D}, OCH₂), 3.92-3.85 (m, 2H, H2_{D}, H3_{E}), 3.83-3.58 (m, 12H, H4_{D}, H5_{D}, H6a_{D}, H6b_{D}, H4_{E}, H5_{E}, H6a_{E}, H6b_{E}, H3_{E′}, H5_{E′}, H6a_{E′}, H6b_{E′}), 3.56 (dd overlapped, 1H, J₁₋₂=4.0 Hz, J₂₋₃=10.0 Hz, H2_{E}), 3.52 (dd overlapped, 1H, J₁₋₂=3.8 Hz, J₂₋₃=9.8 Hz, H2_{E′}), 3.35 (pt, 1H, J₃₋₄=J₄₋₅=9.3 Hz, H4_{E′}), 1.97 (s, 3H, COCH₃); ¹³C NMR (D₂O, 100 MHz) δ (ppm): 174.5 (C═O), 133.7 (CH═), 118.1 (═CH₂), 99.7 (C1_{E}, J_{C1-H1}=171.6 Hz), 99.9 (C1_{E′}, J_{C1-H1}=172.2 Hz), 96.0 (C1_{D}p, J_{C1-H1}=171.5 Hz), 77.7 (C4_{D}), 76.9 (C4_{E}), 73.4 (C3_{E}), 72.9 (C3_{E′}), 72.8 (C5_{E′}), 71.8, 71.6 (2C, C2_{E}, C2_{E′},), 71.5 (C3_{D}), 71.3 (C5_{E}), 70.5 (C5_{D}), 69.4 (C4_{E′}), 68.6 (OCH₂), 60.6 (3C, C6_{D}, C6_{E}, C6_{E′}), 53.5 (C2_{D}), 21.9 (COCH₃). HRMS (ESI⁺) for C₂₃H₃₉NO₁₆Na ([M+Na]⁺, 608.2167) m/z 608.2172.

Allyl 2,3,4,6-tetra-O-acetyl-α-D-glucopyranosyl-(1→4)-2-acetamido-3,6-di-O-acetyl-2-deoxy-α-D-glucopyranoside (XX₄). Acetic anhydride (80 mL, 725 mmol) was added dropwise to a solution of XX₃ in mixture with fructose (4.16 g) in anhydrous pyridine (80 mL). The resulting mixture was stirred at room temperature under argon. After 2 days, TLC showed the complete disappearance of the starting materials. The mixture was concentrated under reduced pressure, and volatiles were eliminated by repeated coevaporation with toluene. The residue was purified by column chromatography (Cyclohexane-EtOAc, 6:4→4:6 then CH₂Cl₂/MeOH 92:2→95:5) to give XX₄ (2.96 g, 30% from XX₁) as a colourless amorphous solid. Compound XX₄ had Rf=0.6 (95:5 CH₂Cl₂-MeOH). ¹H NMR (CDCl₃, 400 MHz) δ (ppm): 5.90 (m, 1H, CH═), 5.71 (d, 1H, J=9.7 Hz, NH), 5.45 (d, 1H, J₁₋₂=4.0 Hz, H1_{E}), 5.35 (dd, 1H, J₃₋₄=9.6 Hz, H3_{E}), 5.34-5.23 (m, 3H, ═CH₂, H3_{D}), 5.04 (pt, 1H, J₄₋₅=9.8 Hz, H4_{E}), 4.85 (dd, 1H, J₂₋₃=10.5 Hz, H2_{E}), 4.79 (d, 1H, J₁₋₂=3.6 Hz, H1_{D}), 4.42 (dd, 1H, J₅₋₆ₐ=2.0 Hz, J_{6a-6b}=12.0 Hz, H6a_{D}), 4.23-4.15 (m, 4H, OCH₂, H2_{D}, H6b_{D}, H6a_{E}), 4.04-3.91 (m, 5H, OCH₂, H4_{D}, H5_{D}, H5_{E}, H6b_{E}), 2.13, 2.08, 2.01, 2.00, 1.98, 1.91 (7s, 21H, COCH₃); ¹³C NMR (CDCl₃, 100 MHz) δ (ppm): 171.6, 170.6, 170.5, 170.2, 170.0, 169.4 (7C, C═O), 133.0 (CH═), 118.7 (═CH₂), 96.1 (C1_{D}), 95.5 (C1_{E}), 74.1 (C3_{D}), 72.4 (C4_{D}), 70.1 (C2_{E}), 69.4 (C3_{E}), 68.8 (OCH₂), 68.4 (C5_{E}), 68.0 (C5_{D}), 67.9 (C4_{E}), 62.8 (C6_{D}), 61.4 (C6_{E}), 52.2 (C2_{D}), 23.1, 21.0, 20.8, 20.7, 20.6, 20.5 (7C, COCH₃). HRMS (ESI⁺) for C₂₉H₄₁NO₁₇Na ([M+Na]⁺, 698.2272) m/z 698.2277.

Allyl α-D-glucopyranosyl-(1→4)-2-acetamido-2-deoxy-α-D-glucopyranoside (XX₃). Method a: Methanolic sodium methoxide (0.5 M solution, 8 mL, 4 mmol) was added to a stirred solution of XX₄ (2.68 g, 3.97 mmol) in anhydrous methanol (100 mL). The reaction mixture was stirred for 6.5 h at room temperature by which time all the starting material had been consumed (Rf=0.3 in 9:1 CH₃CN—H₂O). Excess base was neutralized with Dowex-H⁺ resin. The suspension was filtered, and the filtrate was concentrated under reduced pressure. The residue was purified by silica gel chromatography, eluting with 10% H₂O in CH₃CN to afford compound XX₃ as a brownish foamy solid (1.65 g, 98%); ¹H NMR (D₂O, 400 MHz) δ (ppm): 5.95 (m, 1H, CH═), 5.38 (d, 1H, J₁₋₂=3.9 Hz, H1_{E}), 5.34 (m, 1H, ═CH₂), 5.25 (m, 1H, ═CH₂), 4.91 (d, 1H, J₁₋₂=3.5 Hz, H1_{D}), 4.20 (m, 1H, OCH₂), 4.02 (m overlapped, 1H, OCH₂), 4.01 (dd overlapped, 1H, J₃₋₄=8.4 Hz, H3_{D}), 3.94 (dd, 1H, J₂₋₃=10.8 Hz, H2_{D}), 3.89-3.67 (m, 7H, H4_{D}, H5_{D}, H6a_{D}, H6b_{D}, H5_{E}, H6a_{E}, H6b_{E}), 3.66 (pt, 1H, H3_{E}), 3.56 (dd, 1H, J₂₋₃=9.9 Hz, H2_{E}), 3.40 (pt, 1H, J₃₋₄=J₄₋₅=9.5 Hz, H4_{E}), 2.02 (s, 3H, COCH₃); ¹³C NMR (D₂O, 100 MHz) δ (ppm): 174.5 (C═O), 133.7 (CH═), 118.1 (═CH₂), 99.9 (C1_{E}, J_{C1-H1}=171.3 Hz), 96.0 (C1_{D}, J_{C1-H1}=171.4 Hz), 77.6 (C4_{D}), 72.9 (C3_{E}), 72.8 (C5_{E}), 71.8 (C2_{E}), 71.5 (C3_{D}), 70.6 (C5_{D}), 69.4 (C4_{E}), 68.6 (OCH₂), 60.6 (2C, C6_{D}, C6_{E}), 53.5 (C2_{D}), 21.9 (COCH₃). HRMS (ESI⁺) for C₁₇H₃₀NO₁₁ ([M+H]⁺, 424.1819) m/z 424.1821.

Method b: Trisaccharide XX₂ (2.19 g, 3.74 mmol) in solution in 110 mL of acetate buffer (pH 4.8) was incubated with 1120 IU of amyloglucosidase from Aspergillus niger with magnetic stirring for 90 min at 50° C. After freeze-drying, the products were purified by silica gel chromatography eluting with 9:1 CH₃CN—H₂O. Concentration and freeze-drying gave a 1:1.1 mixture of disaccharide XX₃ and D-glucose (1.43 g, 62%) as seen by NMR.

Method c: To a solution of the crude mixture issued from the enzymatic glucosylation of XX₁ (6.43 g, 24.61 mmol) in anhydrous pyridine (170 mL) was added dropwise anhydride acetic (150 mL, 1.59 mol). The resulting mixture was stirred at room temperature under argon. After 2 days, TLC showed the complete disappearance of the starting materials. The mixture was concentrated under reduced pressure, and volatiles were eliminated by repeated coevaporation with toluene. The residue was purified by column chromatography (Cyclohexane-EtOAc, 6:4 then CH₂Cl₂-MeOH, 97:3) to give a mixture (12.88 g) of peracetylated starting material, intermediate XX₄ and peracetylated trisaccharide resulting from the enzyme-mediated transfer of glucose onto disaccharide XX₂. To a stirred solution of the above mixture (12.88 g) in anhydrous methanol (140 mL) was added sodium methoxide (25% wt solution, 3.2 mL, 11.4 mmol). The reaction mixture was stirred for 23 h at room temperature by which time all the starting material had been consumed (Rf=0.3 in 9:1 CH₃CN—H₂O). Excess base was neutralized with Dowex-H⁺ resin, filtered, concentrated, and purified by silica gel chromatography, eluting with 10% H₂O in CH₃CN to afford compound XX₃ as a brownish foamy solid (4.44 g, 43%).

### EXAMPLE 4

### Chemo-Enzymatic Synthesis of Potential Acceptor Building Blocks to Oligosaccharide Fragments of S. flexneri 1b and/or 1a O-Antigens

Allyl α-D-glucopyranosyl-(1→4)-2-amino-2-deoxy-α-D-glucopyranoside (XX₅).

Method a: Disaccharide XX₃ (784 mg, 1.85 mmol) was treated with Ba(OH)₂.8 H₂O (10 g) in water (50 mL), and the mixture was stirred at 90° C. overnight. When N-deacetylation was completed (Rf=0.5 in 4:1:0.5 iPrOH—H₂O-conc. NH₄OH), the cooled mixture was saturated with CO₂, the volume was diminished to ˜5 mL, EtOH (30 mL) was added, and solids were sedimented by centrifugation (0° C., 666×g, 2000 r.p.m., 10 min.). The supernatant was decanted, the residue was re-extracted with EtOH, and supernatants were pooled. The residue remaining after solvent removal was taken up in 13:7:2 CH₂Cl₂—MeOH-conc. NH₄OH, and passed through a layer (8×3) of silica gel wet with the same solvent. Pooled fractions containing the product were concentrated and freeze-dried to give XX₅ as a solid (587 mg; 83%). ¹H NMR (D₂O, 400 MHz) δ (ppm): 5.88 (m, 1H, CH═), 5.30 (d overlapped, 1H, H1_{E}), 5.27 (m, 1H, ═CH₂), 5.18 (m, 1H, ═CH₂), 4.97 (d, 1H, J₁₋₂=3.7 Hz, H1_{D}), 4.15 (m, 1H, OCH₂), 3.98 (m, 1H, OCH₂), 3.93 (dd, 1H, J₂₋₃=10.5 Hz, J₃₋₄=8.8 Hz, H3_{D}), 3.80-3.62 (m, 5H, H5_{D}, H6a_{D}, H6b_{D}, H6a_{E}, H6b_{E}), 3.62-3.57 (m, 2H, H4_{D}, H5_{E}), 3.57 (dd overlapped, 1H, J₃₋₄=9.1 Hz, H3_{E}), 3.48 (dd, 1H, J₁₋₂=4.0 Hz, J₂₋₃=9.9 Hz, H2_{E}), 3.31 (pt, 1H, J₄₋₅=9.6 Hz, H4_{E}), 3.03 (dd, 1H, H2_{D}); ¹³C NMR (D₂O, 100 MHz) δ (ppm): 133.4 (CH═), 118.5 (═CH₂), 99.7 (C1_{E}), 95.8 (C1_{D}), 76.7 (C4_{D}), 72.9 (C5_{E}*), 72.8 (C3_{E}*), 72.1 (C3_{D}), 71.7 (C2_{E}), 70.7 (C5_{D}), 69.4 (C4_{E}), 68.8 (OCH₂), 60.5, 60.4 (2C, C6_{D}, C6_{E}), 54.2 (C2_{D}). HRMS (ESI⁺) for C₁₅H₂₈NO₁₀ ([M+H]⁺, 382.1713) m/z 382.1719.

Method b: One-pot removal of O-acetyl and N-acetamide groups in disaccharide XX₄ followed the above mentioned procedure. Briefly, XX₄ (5.05 g, 7.47 mmol) was treated overnight with Ba(OH)₂.8 H₂O (25 g) in water (50 mL) at 90° C. After neutralization with dry ice and repeated centrifugations (0° C., 666×g, 2000 r.p.m., 10 min.), compound XX₅ was purified by silica gel chromatography eluting with 13:7:2 CH₂Cl₂-MeOH-conc. NH₄OH (2.39 g, 84%).

Allyl α-D-glucopyranosyl-(1→4)-2-amino-2-N,3-O-carbonyl-2-deoxy-α-D-glucopyranoside (XX₆). An ice-cooled solution of p-nitrophenoxycarbonyl chloride (2.50 g, 12.6 mmol) in acetone (25 mL) was added over several minutes to a stirred, ice bath-cooled, mixture of XX₅ (1.60 g, 4.2 mmol) and methanolic NaOMe (25% w/w solution, 3.5 mL, 12.6 mmol) in 50 mL methanol. The mixture was vigorously stirred with ice-bath cooling for 30 min, then at room temperature for 1 h. Water (100 mL) was added and the resulting aqueous mixture was extracted with diethyl ether. The aqueous layer was made acidic (pH˜3) by dropwise addition of 10% hydrochloric acid solution. The acidified aqueous phase was extracted with diethyl ether to remove p-nitrophenol until the etheral layer failed to turn yellow upon NaHCO₃ addition. The aqueous solution was then neutralized with Amberlyst-A26 (OH⁻ form) resin, filtered, and evaporated to give crude oxazolidinone) XX₆ (1.70 g). ¹H NMR (CD₃OD, 400 MHz) δ (ppm): 5.98 (m, 1H, CH═), 5.37 (m, 1H, ═CH₂), 5.29 (d, 1H, J₁₋₂=3.8 Hz, H1_{E}), 5.22 (m, 1H, ═CH₂), 5.17 (d, 1H, J₁₋₂=2.9 Hz, H1_{D}), 4.78 (dd, 1H, J₂₋₃=12.0 Hz, J₃₋₄=9.8 Hz, H3_{D}), 4.30 (m, 1H, OCH₂), 4.29 (pt overlapped, 1H, H4_{D}), 4.13 (m, 1H, OCH₂), 3.86 (dd, 1H, J₅₋₆ₐ=2.3 Hz, J_{6a-6b}=11.8 Hz, H6a_{E}), 3.83 (dd, 2H, J₅₋₆=3.1 Hz, H6a_{D}, H6b_{D}), 3.77 (dt, 1H, J₄₋₅=9.3 Hz, H5_{D}), 3.72 (dd, 1H, J_{5-6b}=5.4 Hz, H6b_{E}), 3.68 (dd, 1H, H2_{D}), 3.65-3.59 (m overlapped, 2H, H3_{E}, H5_{E}), 3.46 (dd, 1H, J₂₋₃=9.8 Hz, H2_{E}), 3.35 (dd, 1H, J₃₋₄=9.0 Hz, J₄₋₅=9.8 Hz, H4_{E}); ¹³C NMR (CD₃OD, 100 MHz) δ (ppm): 160.6 (C═O), 133.7 (CH═), 116.3 (═CH₂), 97.4 (C1_{E}), 94.8 (C1_{D}), 79.5 (C3_{D}), 73.3 (C3_{E}*), 73.1 (2C, C5_{D}, C5_{E}*), 71.8 (C2_{E}), 71.0 (C4_{D}), 70.2 (C4_{E}), 68.5 (OCH₂), 61.1 (C6_{E}), 60.2 (C6_{D}), 58.4 (C2_{D}). HRMS (ESI⁺) for C₁₆H₂₅NO₁₁Na ([M+Na]⁺, 430.1325) m/z 430.1331.

Allyl 2,4,6-tri-O-benzyl-α-D-glucopyranosyl-(1→4)-6-O-benzyl-2-benzylamino-2-N,3-O-carbonyl-2-deoxy-α-D-glucopyranoside (XX₇). 60% NaH in oil (57 mg, 1.42 mmol) was added portionwise to an ice-cold mixture of the crude oxazolidinone XX₆ (108 mg) and benzyl bromide (0.19 mL, 1.60 mmol) in DMF (5 mL). After stirring for 30 min at 0° C., the reaction mixture was warmed up and stirred for an additional 5.5 h at room temperature. The reaction was quenched by addition of Et₃N, diluted with EtOAc, and the mixture was washed with brine, dried (Na₂SO₄), filtered, and concentrated. The residue was purified by flash-chromatography (4:1 Cyclohexane-EtOAc) to afford XX₈ (48 mg, 19% over 2 steps) and the corresponding 3-hydroxyl compound XX₇ (39 mg, 17% over two steps); Rf=0.2 (3:1 Cyclohexane-EtOAc). ¹H NMR (CDCl₃, 400 MHz) δ (ppm): 7.45-7.22 (m, 25H, H arom.), 5.76 (m, 1H, CH═), 5.54 (d, 1H, J₁₋₂=3.6 Hz, H1_{E}), 5.30-5.20 (m, 2H, ═CH₂), 4.86 (d overlapped, 1H, CH₂Ph), 4.84 (d overlapped, 1H, CH₂Ph), 4.76 (d, 1H, J₁₋₂=2.8 Hz, H1_{D}), 4.65 (dd overlapped, 1H, H3_{D}), 4.64 (d overlapped, 1H, CH₂Ph), 4.54 (d overlapped, 1H, CH₂Ph), 4.52 (d overlapped, 1H, CH₂Ph), 4.48 (m, 2H, CH₂Ph), 4.41 (m, 2H, CH₂Ph), 4.33 (d overlapped, 1H, CH₂Ph), 4.30 (pt overlapped, 1H, J₃₋₄==9.6 Hz, H4_{D}), 4.02 (m overlapped, 1H, OCH₂), 4.00 (pt overlapped, 1H, H3_{E}), 3.81 (ddd, 1H, H5_{D}), 3.76 (dd, 1H, J₅₋₆ₐ=3.7 Hz, J_{6a-6b}=11.0 Hz, H6a_{D}), 3.70-3.57 (m, 4H, OCH₂, H6b_{D}, H4_{E}, H5_{E}), 3.54 (dd, 1H, J₅₋₆ₐ=2.4 Hz, J_{6a-6b}=10.7 Hz, H6a_{E}), 3.65 (dd 1H, J₂₋₃=9.7 Hz, H2_{E}), 3.41 (dd overlapped, 1H, J_{5-6b}=1.2 Hz, H6b_{E}), 3.34 (dd, 1H, J₂₋₃=11.8 Hz, H2_{D}); ¹³C NMR (CDCl₃, 100 MHz) δ (ppm): 158.6 (C═O), 138.6, 138.0, 137.8, 137.6, 135.3 (5C, C quat. arom.), 133.1 (CH═), 128.8, 128.7, 128.6, 128.5, 128.3, 128.2, 128.1, 127.9, 127.8, 127.6, 127.3 (25C, C arom.), 118.4 (═CH₂), 94.8 (C1_{E}), 94.3 (C1_{D}), 78.7 (C2_{E}), 77.2 (C4_{E}), 76.9 (C3_{D}), 74.6, 73.5 (3C, OCH₂Ph), 73.2 (C3_{E}), 72.5 (OCH₂Ph), 72.1 (C5_{D}), 71.0 (C4_{D}), 70.9 (C5_{E}), 69.1 (OCH₂), 68.3 (C6_{D}), 68.1 (C6_{E}), 61.1 (C2_{D}), 48.7 (NCH₂Ph). HRMS (ESI⁺) for C₅₁H₅₅NO₁₁Na ([M+Na]⁺, 880.3673) m/z 880.3704.

Allyl 2,3,4,6-tetra-O-benzyl-α-D-glucopyranosyl-(1→4)-6-O-benzyl-2-benzylamino-2-N,3-O-carbonyl-2-deoxy-α-D-glucopyranoside (XX₈). Methanolic sodium methoxide (0.5 M solution, 1.6 mL, 0.79 mmol) and trichloroethylchloroformate (225 μL, 1.56 mmol) were added to a solution of compound XX₅ (100 mg, 0.26 mmol) in methanol (3.4 mL) at 0° C. After 4 h, the reaction mixture was neutralized by addition of Dowex-H⁺ resin, filtered, and concentrated to dryness to afford the crude trichloroethyl carbamate. This material (Rf=0.7, 85:15 CH₃CN—H₂O) was used directly without further purification.

NaH (60% in oil, 63 mg, 2.60 mmol) was added to an ice-cold mixture of the crude trichloroethyl carbamate and benzyl bromide (0.31 mL, 2.60 mmol) in DMF (5 mL). The reaction mixture was stirred for 30 min at 0° C., then stirred for an additional 4.5 h at room temperature. The reaction was quenched by addition of Et₃N, diluted with EtOAc, poured into saturated aqueous NH₄Cl, and extracted with EtOAc. The combined organic extracts were washed with water and brine, dried (Na₂SO₄), filtered, and concentrated. The residue was purified by flash-chromatography (17:3 Cyclohexane-EtOAc) to afford XX₈ (81 mg, 34% over 2 steps); Rf=0.3 (4:1 Cyclohexane-EtOAc). ¹H NMR (CDCl₃, 400 MHz) δ (ppm): 7.46-7.16 (m, 30H, H arom.), 5.79 (m, 1H, CH═), 5.56 (d, 1H, J₁₋₂=3.5 Hz, H1_{E}), 5.26 (m, 2H, ═CH₂), 5.02 (d overlapped, 1H, CH₂Ph), 4.87-4.80 (m, 3H, CH₂Ph), 4.79-4.72 (m overlapped, 3H, H1_{D}, H3_{D}, CH₂Ph), 4.56-4.49 (m, 4H, CH₂Ph), 4.47 (m, 2H, CH₂Ph), 4.35 (pt overlapped, 1H, H4_{D}), 4.34 (d overlapped, 1H, CH₂Ph), 4.05 (m, 1H, OCH₂), 3.92-3.88 (m, 2H, H5_{D}, H3_{E}), 3.81 (dd, 1H, J₅₋₆ₐ=3.5 Hz, J_{6a-6b}=11.0 Hz, H6a_{D}), 3.73-3.66 (m, 4H, OCH₂, H6b_{D}, H4_{E}, H5_{E}), 3.65 (dd overlapped, 1H, H2_{E}), 3.57 (dd, 1H, J₅₋₆ₐ=2.7 Hz, J_{6a-6b}=10.8 Hz, H6a_{E}), 3.43 (dd, 1H, J_{5-6b}=1.4 Hz, H6b_{E}), 3.37 (dd, 1H, J₁₋₂=2.7 Hz, J₂₋₃=11.8 Hz, H2_{D}); ¹³C NMR (CDCl₃, 100 MHz) δ (ppm): 158.7 (C═O), 138.9, 138.5, 138.1, 137.9, 137.7, 135.3 (6C, C quat. arom.), 133.1 (CH═), 128.8, 128.7, 128.5, 128.4, 128.3, 128.2, 127.9, 127.8, 127.6, 127.5, 127.3 (30C, C arom.), 118.3 (═CH₂), 95.4 (C1_{E}), 94.4 (C1_{D}), 81.8 (C3_{E}), 79.5 (C2_{E}), 77.4 (C4_{E}), 76.9 (C3_{D}), 75.6, 75.1, 73.5, 73.1 (5C, OCH₂Ph), 72.1 (C5_{D}), 71.3 (C5_{E}), 70.9 (C4_{D}), 69.1 (OCH₂), 68.4 (C6_{D}), 68.2 (C6_{E}), 61.2 (C2_{D}), 48.7 (NCH₂Ph). HRMS (ESI⁺) for C₅₈H₆₁NO₁₁Na ([M+Na]⁺, 970.4142) m/z 970.4107.

Allyl 2,3,4,6-tetra-O-benzyl-α-D-glucopyranosyl-(1→4)-6-O-benzyl-2-amino-2-N,3-O-carbonyl-2-deoxy-α-D-glucopyranoside (XX_{8A}). NaH (60% in oil, 368 mg, 9.21 mmol) was added portionwise to a stirred solution of the crude oxazolidinone XX₆ (500 mg, 1.23 mmol) in dry DMF (10 mL) at 0° C. After 1 h, the mixture was treated with benzyl bromide (800 μL, 6.75 mmol), stirred overnight, treated with MeOH, and concentration to dryness. The residue was purified by flash-chromatography (85:15 Toluene-EtOAc) to afford XX₈ (74 mg, 6% over 2 steps), and the corresponding penta-O-benzyl derivative XX_{8A} (158 mg, 15%); Rf=0.3 (85:15 Toluene-EtOAc). ¹H NMR (CDCl₃, 400 MHz) δ (ppm): 7.35-7.17 (m, 25H, H arom.), 5.80 (m, 1H, CH═), 5.57 (d, 1H, J₁₋₂=3.5 Hz, H1_{E}), 5.21 (m, 1H, ═CH₂), 5.13 (m, 2H, ═CH₂), 4.95 (d, 1H, CH₂Ph), 4.86 (d overlapped, 1H, CH₂Ph), 4.83 (d overlapped, 1H, CH₂Ph), 4.78 (d overlapped, 1H, CH₂Ph), 4.77 (d, 1H, J₁₋₂=3.6 Hz, H1_{D}), 4.71-4.49 (m, 5H, CH₂Ph), 4.37 (d overlapped, 1H, CH₂Ph), 4.29 (ddd, 1H, J₂₋₃=10.1 Hz, H2_{D}), 4.19 (pt, 1H, J₃₋₄=J₄₋₅=9.2 Hz, H4_{D}), 4.06 (m, 1H, OCH₂), 3.97 (pt overlapped, 1H, H3_{E}), 3.95 (dd overlapped, 1H, H6a_{D}), 3.91-3.81 (m, 3H, H3_{D},H5_{D}, H5_{E}), 3.76-3.64 (m, 3H, OCH₂, H6b_{D}, H4_{E}), 3.57 (dd, 1H, J₅₋₆ₐ=3.2 Hz, J_{6a-6b}=10.8 Hz, H6a_{E}), 3.52 (dd 1H, J₂₋₃=9.8 Hz, H2_{E}), 3.48 (dd, 1H, J_{5-6b}=1.4 Hz, H6b_{E}); ¹³C NMR (CDCl₃, 100 MHz) δ (ppm): 156.4 (C═O), 138.8, 138.6, 138.4, 138.1, 135.3 (5C, C quat. arom.), 133.8 (CH═), 128.3, 128.1, 128.0, 127.9, 127.8, 127.6, 127.5, 127.4 (25C, C arom.), 117.6 (═CH₂), 97.4 (C1_{D}), 96.7 (C1_{E}), 82.0 (C3_{E}), 80.5 (C3_{D}), 79.5 (C2_{E}), 77.7 (C4_{E}), 75.6, 75.0, 73.2, 72.9 (4C, OCH₂Ph), 72.6 (C4_{D}), 71.7 (OCH₂Ph), 71.1 (C5_{E}), 70.7 (C5_{D}), 69.0 (C6_{D}), 68.4 (C6_{E}), 68.4 (OCH₂), 52.8 (C2_{D}). HRMS (ESI⁺) for C₅₁H₅₅NO₁₁Na ([M+Na]⁺, 880.3673) m/z 880.3710.

Allyl 2,3,4,6-tetra-O-benzyl-α-D-glucopyranosyl-(1→4)-6-O-benzyl-2-benzylamino-2-deoxy-α-D-glucopyranoside (XX₉). Perbenzylated XX₈ (36 mg, 0.04 mmol) was treated with 1 M aqueous NaOH/1,4-dioxane (1:1, v/v, 10 mL) at 65° C. for 2 days. The mixture was diluted with EtOAc and washed with water. The separated aqueous layer was extracted with EtOAc. The combined organic extracts were washed with water and brine, dried (Na₂SO₄), filtered and concentrated. The residue was purified by flash-chromatography (4:1 Cyclohexane-EtOAc) to afford target XX₉ (32 mg, 91%) as an oil; Rf=0.1 (4:1 Cyclohexane-EtOAc). ¹H NMR (CDCl₃, 400 MHz) δ (ppm): 7.41-7.16 (m, 30H, H arom.), 5.95 (m, 1H, CH═), 5.33 (m, 1H, ═CH₂), 5.24 (d overlapped, 1H, J₁₋₂=3.4 Hz, H1_{E}), 5.23 (m, 1H, ═CH₂), 4.96 (d, 1H, CH₂Ph), 4.90-4.76 (m, 4H, CH₂Ph, H1_{D}), 4.57 (d overlapped, 1H, CH₂Ph), 4.55 (d overlapped, 1H, CH₂Ph), 4.50 (d overlapped, 1H, CH₂Ph), 4.46 (d overlapped, 1H, CH₂Ph), 4.40 (d overlapped, 1H, CH₂Ph), 4.16 (m, 1H, OCH₂), 4.00 (pt overlapped, 1H, H3_{E}), 3.99-3.95 (m, 4H, OCH₂, CH₂Ph, H3_{D}), 3.85-3.77 (m, 2H, H5_{D}, H5_{E}), 3.76-3.63 (m, 4H, H4_{D}, H4_{E}, H6a_{D}, H6b_{D}), 3.62-3.56 (m, 2H, H2_{E}, H6a_{E}), 3.47 (dd, 1H, J_{5-6b}=1.8 Hz, J_{6a6b}=10.6 Hz, H6b_{E}), 2.80 (dd, 1H, J₁₋₂=3.5 Hz, J₂₋₃=10.2 Hz, H2_{D}); ¹³C NMR (CDCl₃, 100 MHz) δ (ppm): 138.7, 138.4, 138.3, 137.9, 135.4 (6C, C quat. arom.), 134.0 (CH═), 128.6, 128.5, 128.4, 128.3, 128.1, 127.9, 127.8, 127.7, 127.6, 127.5, 127.4, 127.1 (30C, C arom.), 117.7 (═CH₂), 99.4 (C1_{E}), 96.3 (C1_{D}), 82.1 (C3_{E}), 80.3 (C4_{D}), 79.7 (C2_{E}), 77.7 (C4_{E}), 75.7, 75.0, 73.7, 73.5, 73.2 (5C, OCH₂Ph), 73.2 (C3_{D}), 71.2 (C5_{E}), 69.5 (C5_{D}), 69.1 (C6_{D}), 68.5 (C6_{E}), 68.4 (OCH₂), 61.1 (C2_{D}), 51.9 (NCH₂Ph). HRMS (ESI⁺) for C₅₇H₆₄NO₁₀ ([M+H]⁺, 922.4530) m/z 922.4540.

Allyl 2,3,4,6-tetra-O-benzyl-α-D-glucopyranosyl-(1→4)-6-O-benzyl-2-benzylacetamido-2-deoxy-α-D-glucopyranoside (XX₁₀).

Method a: To a solution of disaccharide XX₉ (424 mg, 0.46 mmol) in anhydrous pyridine (5 mL) was added dropwise anhydride acetic (250 μL, 2.65 mmol). The resulting mixture was stirred at room temperature under argon. After 24 h, TLC showed the complete disappearance of the starting materials. The mixture was concentrated under reduced pressure, and volatiles were eliminated by repeated coevaporation with toluene. The residue was purified by column chromatography (Cyclohexane-EtOAc, 2:1) to give XX₁₀, (391 mg, 88%) as a colourless oil. Compound XX₁₀ had Rf=0.2 (2:1 Cyclohexane-EtOAc). NMR analysis indicated an equilibrium between two rotamers.

Method b: Perbenzylated XX₈ (125 mg, 0.13 mmol) was treated with 1 M aqueous NaOH/1,4-dioxane (1:1, v/v, 15 mL) at 65° C. for 2 days. The mixture was diluted with EtOAc and washed with water. The separated aqueous layer was extracted with EtOAc. The combined organic extracts were washed with water and brine, dried (Na₂SO₄), filtered, and concentrated to give crude XX₉ (109 mg, 0.12 mmol) as an oil.

Anhydride acetic (56 μL, 0.59 mmol) was added to a solution of crude disaccharide XX₉ (109 mg, 0.12 mmol) in anhydrous pyridine (1 mL). The mixture was stirred at room temperature under argon. After 5 h, TLC showed the complete disappearance of the starting materials. Volatiles were eliminated by repeated coevaporation with toluene under reduced pressure. The residue was purified by column chromatography (Cyclohexane-EtOAc, 2:1) to give XX₁₀ (98 mg, 77%) as a colourless oil. ¹H NMR (CDCl₃, 400 MHz) δ (ppm): 7.37-7.15 (m, 30H, H arom.), 5.50-5.40 (m, 1H, CH═), 5.10-4.76 (m, 7H, H1_{D}, H1_{E}, H2_{D}, ═CH₂, OCH₂Ph), 4.75-4.65 (m, 3H, OCH₂Ph, NCH₂Ph), 4.60-4.38 (m, 5H, OCH₂Ph), 4.27-4.12 (m, 0.9H, H3_{D}, OCH₂), 4.04-3.91 (m, 2.1H, H3_{D}, OCH₂), 3.91-3.81 (m, 1H, H5_{E}), 3.80-3.50 (m, 6H, H2_{E}, H4_{D}, H4_{E}, H6a_{D}, H6b_{D}, H6a_{E}), 3.50-3.38 (m, 1H, H6b_{E}), 2.31 (s, 0.9H, COCH₃), 1.99 (s, 2.1H, COCH₃); ¹³C NMR (CDCl₃, 100 MHz) δ (ppm): 173.9, 172.5 (C═O), 139.8, 139.4, 138.5, 138.2, 137.8, 136.9 (C quat. arom.), 133.5 (CH═), 128.8, 128.7, 128.6, 128.4, 128.3, 128.0, 127.9, 127.7, 127.6, 127.5, 127.4, 126.6, 126.4, 125.9 (C arom.), 117.8, 117.3 (═CH₂), 100.8 (C1_{E}), 98.0, 97.7 (C1_{D}), 83.8 (C4_{D}), 82.2 (C3_{E}), 79.4 (C2_{E}), 77.7 (C4_{E}), 75.7, 75.6, 75.0, 74.2, 73.5, 73.3, 73.1 (OCH₂Ph), 71.3 (C5_{E}), 70.0 (C5_{D}), 69.1 (C6_{D}), 69.0 (C3_{D}), 68.9 (OCH₂), 68.3 (C6_{E}), 56.4 (C2_{D}), 48.5 (NCH₂Ph), 22.6 (COCH₃). HRMS (ESI⁺) for C₅₉H₆₆NO_{I} ([M+H]⁺, 964.4636) m/z 964.4813.

Allyl 2-O-benzoyl-4-O-benzyl-3-O-chloroacetyl-α-L-rhamnopyranosyl-(1→3)[2,3,4,6-tetra-O-benzyl-α-D-glucopyranosyl-(1→4)]-6-O-benzyl-2-benzylacetamido-2-deoxy-α-D-glucopyranoside (XX₁₁). TMSOTf (5.5 μL, 0.030 mmol) was added to a solution of known 2-O-benzoyl-4-O-benzyl-3-β-chloroacetyl-α-L-rhamnopyranosyl trichloroacetimidate (ref. 73) (73 mg, 0.126 mmol) and acceptor XX₁₀ (98 mg, 0.102 mmol) in toluene (2.75 mL) containing activated MS4 Å under argon at −10° C. The mixture was stirred at room temperature and, when TLC monitoring indicated complete consumption of the donor, the reaction was quenched by addition of triethylamine. After filtration through a bed of celite and concentration under vacuum, flash chromatography (85:15 Toluene-EtOAc) gave a mixture of two products (Rf=0.4, 4:1 Toluene-EtOAc), among which the target trisaccharide XX₁₁ as indicated by mass spectrometry analysis. Compound XX₁₁ had HRMS (ESI⁺) for C₈₁H₈₆ClNO₁₇Na ([M+Na]⁺, 1402.5482) m/z 1402.5525.

Allyl 2-O-acetyl-3,4-di-O-benzyl-α-L-rhamnopyranosyl-(1→3)-[2,3,4,6-tetra-O-benzyl-α-D-glucopyranosyl-(1→4)]-6-O-benzyl-2-benzylacetamido-2-deoxy-α-D-glucopyranoside (XX₁₂). TMSOTf (4 μL, 0.026 mmol) was added to a solution of known 2-O-acetyl-3,4-di-O-benzyl-α-L-rhamnopyranosyl trichloroacetimidate (ref. 74) (132 mg, 0.249 mmol) and acceptor XX₁₀ (76 mg, 0.079 mmol) in toluene (4 mL) containing activated MS4 Å, stirred under argon at 0° C. Following additional stirring for 3 h at 60° C., TLC monitoring indicated complete consumption of the donor. The reaction was quenched by addition of triethylamine. After filtration through a bed of celite and concentration under vacuum, flash chromatography (9:1 Toluene-EtOAc) gave the expected trisaccharide XX₁₂ (Rf=0.4, 4:1 Toluene-EtOAc). ¹H NMR (CDCl₃, 400 MHz) δ (ppm): 7.28-7.12 (m, 41H, NH, H arom.), 5.61 (d, 1H, J₁₋₂=3.6 Hz, H1_{E}), 5.22 (m, 1H, H2_{D}), 5.14 (pt, 1H, J₁₋₂=J₂₋₃=2.5 Hz, H2_{C}), 5.08-4.97 (m, 1H, CH═), 4.83 (d overlapped, 1H, H1_{C}), 4.83-4.57 (m, 11H, H1_{D}, 2 ═CH₂, 6 OCH₂Ph, 2 NCH₂Ph), 4.50-4.32 (m, 7H, 7 OCH₂Ph), 4.30-4.20 (m, 2H, H3_{D}, OCH₂Ph), 4.09 (dd, 1H, J₃₋₄=7.2 Hz, J₄₋₅=9.0 Hz, H4_{D}), 4.01-3.90 (m, 2H, H5c, OCH₂), 3.85 (dd overlapped, 1H, H3_{C}), 3.83-3.60 (m, 6H, H5_{D}, H3_{E}, H4_{E}, H5_{E}, H6a_{D}, OCH₂), 3.56-3.42 (m, 2H, H6b_{D}, H6a_{E}), 3.40 (dd, 1H, J₂₋₃=9.6 Hz, H2_{E}), 3.33 (pt, 1H, J₃₋₄=J₄₋₅=9.3 Hz, H4_{C}), 3.09 (m, 1H, J₅₋₆=5.7 Hz, J_{6a-6b}=11.8 Hz, H6b_{E}), 2.04, 1.98 (2s, 6H, COCH₃), 1.20 (d, 3H, J₅₋₆=6.1 Hz, H6_{C}); ¹³C NMR (CDCl₃, 100 MHz) δ (ppm): 173.8, 170.4 (C═O), 139.0, 138.9, 138.7, 138.6, 138.4, 138.3 (C quat. arom.), 133.1 (CH═), 128.5, 128.4, 128.3, 128.2, 128.1, 127.9, 127.8, 127.7, 127.6, 127.4, 127.3, 126.6, 125.3 (C arom.), 117.5 (═CH₂), 98.1 (C1_{C}), 97.5 (C1_{D}), 94.2 (C1_{E}), 81.8 (C3_{E}), 80.2 (C2_{E}), 79.8 (C4_{C}), 79.7 (C3_{D}), 77.7 (C4_{E}), 77.6 (C3_{C}), 75.4, 75.0, 74.8, 73.4 (4C, OCH₂Ph), 73.2 (C4_{D}), 73.2, 73.1, 71.6 (3C, OCH₂Ph), 71.3 (C5_{D}*), 70.2 (C2_{C}), 70.1 (C5_{E}*), 69.4 (C6_{D}), 68.8 (OCH₂), 68.7 (C5_{C}), 68.6 (C6_{E}), 54.9 (C2_{D}), 48.3 (NCH₂Ph), 22.7, 21.1 (2C, COCH₃), 18.1 (C6_{C}). HRMS (ESI⁺) for C₈₁H₈₉NO₁₆Na ([M+Na]⁺, 1354.6079) m/z 1354.5994.

### EXAMPLE 5

### Chemo-Enzymatic Synthesis of Potential Donor Building Blocks to Oligosaccharide Fragments of S. flexneri 1b and/or 1a O-Antigens

Allyl α-D-glucopyranosyl-(1→4)-2-deoxy-2-trichloroacetamido-α-D-glucopyranoside (XX₁₃). Methanolic sodium methoxide (25% wt solution in MeOH, 1.72 mL, 6.10 mmol) and trichloracetic anhydride (560 μL, 3.07 mmol) were added to a solution of compound XX₅ (390 mg, 1.02 mmol) in methanol (8 mL) at 0° C. After 1 h the reaction mixture was neutralized by addition of Dowex-H⁺ resin, filtered, and concentrated to dryness to obtain crude XX₁₃. This material was used directly without further purification. Compound XX₁₃ had Rf=0.7 (85:15 CH₃CN—H₂O). HRMS (ESI⁺) of C₁₇H₂₆Cl₃NO₁₁Na ([M+Na]⁺, 548.0469) m/z 548.0524.

Allyl 2,3,4,6-tetra-O-acetyl-α-D-glucopyranosyl-(1→4)-3,6-di-O-acetyl-2-deoxy-2-trichloroacetamido-α-D-glucopyranoside (XX₁₄). Acetic anhydride (5 mL, 52.9 mmol) was added dropwise to a solution of crude XX₁₃ (1.02 mmol) in anhydrous pyridine (5 mL) and the resulting mixture was stirred overnight at room temperature. The reaction was quenched at 0° C. by addition of methanol, and the mixture was evaporated to dryness. The residue was purified by flash-chromatography (3:1 Cyclohexane-Acetone) to yield to XX₁₄ (458 mg, 58% over 2 steps) as an oil. Compound XX₁₄ had Rf=0.5 (3:1 Cyclohexane-Acetone). ¹H NMR (CDCl₃, 400 MHz) δ (ppm): 6.93 (d, 1H, J=9.1 Hz, NH), 5.92 (m, 1H, CH═), 5.51 (d, 1H, J₁₋₂=4.0 Hz, H1_{E}), 5.47-5.27 (m, 4H, ═CH₂, H3_{D}, H3_{E}), 5.09 (pt, 1H, J₄₋₅=10.0 Hz, H4_{E}), 4.95 (d, 1H, J₁₋₂=3.6 Hz, H1_{D}), 4.89 (dd, 1H, J₂₋₃=10.5 Hz, H2_{E}), 4.48 (dd, 1H, J₅₋₆ₐ=1.3 Hz, J_{6a-6b}=12.0 Hz, H6a_{D}), 4.31-4.23 (m, 3H, OCH₂, H6b_{D}, H6a_{E}), 4.14 (ddd overlapped, 1H, J₂₋₃=10.8 Hz, H2_{D}), 4.11-4.05 (m, 4H, OCH₂, H4_{D}, H5_{D}, H6b_{E}), 3.99 (pdt, 1H, J₅₋₆ₐ=3.1 Hz, J_{5-6b}=10.0 Hz, H5_{E}), 2.18, 2.13, 2.06, 2.05, 2.02 (6s, 18H, COCH₃); ¹³C NMR (CDCl₃, 100 MHz) δ (ppm): 171.4, 170.5, 169.9, 169.4 (6C, C═O), 161.9 (C═O), 132.7 (CH═), 119.2 (═CH₂), 95.6 (C1_{E}), 95.2 (C1_{D}), 92.0 (CCl₃), 73.1 (C3_{D}), 72.3 (C4_{D}), 70.1 (C2_{E}), 69.4 (C3_{E}), 69.1 (OCH₂), 68.6 (C5_{E}), 68.3 (C5_{D}), 68.0 (C4_{E}), 62.7 (C6_{D}), 61.4 (C6_{E}), 54.4 (C2_{D}), 21.0, 20.8, 20.7, 20.6, 20.5 (6C, COCH₃). HRMS (ESI⁺) of C₂₉H₃₈Cl₃NO₁₇Na ([M+Na]⁺, 800.1103) m/z 800.1112.

2,3,4,6-tetra-O-acetyl-α-D-glucopyranosyl-(1→4)-3,6-di-O-acetyl-2-deoxy-2-trichloroacetamido-α-D-glucopyranose (XX₁₅). A catalytic amount of 1,5-cyclooctadiene-bis[methyldiphenylphosphine]-iridium hexafluorophosphate (50 mg) was dissolved in dry THF (20 mL). The stirred solution was degassed, placed under hydrogen for 10 minutes until the orange colour turned yellow, degassed and placed under nitrogen. A solution of XX₁₄ (444 mg, 0.57 mmol) in THF/H₂O mixture (5:2, v/v, 7 mL), was then poured into the solution of iridium complex. The reaction mixture was stirred for 3 h and I₂ (290 mg, 1.14 mmol) was added. After stirring for another 3 h, the mixture was diluted with dichloromethane, washed with aqueous NaHSO₃ and water, dried and concentrated. The crude material was used as such. Compound XX₁₅ had Rf=0.2 (3:2 Cyclohexane-EtOAc). ¹H NMR (CDCl₃, 400 MHz) δ (ppm): 7.03 (d, 1H, J=9.2 Hz, NH), 5.53 (d overlapped, 1H, H1_{E}), 5.50 (dd overlapped, 1H, J₂₋₃=10.8 Hz, J₃₋₄=9.0 Hz, H3_{D}), 5.39 (pt, 1H, J₂₋₃=J₃₋₄=10.0 Hz, H3_{E}), 5.31 (sl, 1H, H1_{D}), 5.10 (pt, 1H, J₄₋₅=10.0 Hz, H4_{E}), 4.89 (dd, 1H, J₁₋₂=4.0 Hz, H2_{E}), 4.52 (dd, 1H, J₅₋₆ₐ=3.7 Hz, J_{6a-6b}=13.4 Hz, H6a_{D}), 4.32-4.23 (m, 3H, H5_{D}, H6b_{D}, H6a_{E}), 4.15-3.94 (m, 4H, H2_{D}, H4_{D}, H5_{E}, H6b_{E}), 2.18, 2.13, 2.06, 2.05, 2.02 (6s, 18H, COCH₃). ¹³C NMR (CDCl₃, 100 MHz) δ (ppm): 171.4, 170.6, 170.0, 169.4 (6C, C═O), 162.0 (C═O), 95.5 (C1_{E}), 92.0 (CCl₃), 90.6 (C1_{D}), 72.9 (C3_{D}), 72.3 (C4_{D}), 69.9 (C2_{E}), 69.4 (C3_{E}), 68.5 (C5_{E}), 68.0 (2C, C5_{D}, C4_{E}), 62.8 (C6_{D}), 61.4 (C6_{E}), 54.7 (C2_{D}), 21.0, 20.8, 20.7, 20.6, 20.5 (6C, COCH₃). HRMS (ESI⁺) of C₂₆H₃₄Cl₃NO₁₇Na ([M+Na]⁺, 760.0709) m/z 760.0804.

2,3,4,6-Tetra-O-acetyl-α-D-glucopyranosyl-(1→4)-3,6-di-O-acetyl-2-deoxy-2-trichloroacetamido-α-D-glucopyranosyl trichloroacetimidate (XX₁₆). DBU (26 μL, 0.17 mmol) was added to a stirred solution of crude XX₁₅ (0.57 mmol) and CCl₃CN (70 μL, 0.68 mmol) in dichloroethane (6 mL) at −5° C. After 3 h, the solution was concentrated. Column chromatography (2:1 Cyclohexane-EtOAc) of the residue yielded XX₁₆ (131 mg, 26%) as a syrup. Compound XX₁₆ had Rf=0.4 (3:2 Cyclohexane-EtOAc). ¹H NMR (CDCl₃, 400 MHz) δ (ppm): 6.98 (d, 1H, J=8.7 Hz, NH), 6.46 (d, 1H, J₁₋₂=3.6 Hz, H1_{D}), 5.56 (d, 1H, J₁₋₂=4.1 Hz, H1_{E}), 5.51 (dd overlapped, 1H, H3_{D}), 5.41 (dd overlapped, 1H, H3_{E}), 5.10 (pt, 1H, J₄₋₅=10.0 Hz, H4_{E}), 4.89 (dd, 1H, J₂₋₃=10.4 Hz, H2_{E}), 4.50 (dd, 1H, J₅₋₆ₐ=1.8 Hz, J_{6a-6b}=12.4 Hz, H6a_{D}), 4.38-4.24 (m, 4H, H2_{D}, H5_{D}, H6b_{D}, H6a_{E}), 4.20-3.94 (m, 3H, H4_{D}, H5_{E}, H6b_{E}), 2.18, 2.13, 2.06, 2.10, 2.05, 2.02 (6s, 18H, COCH₃).

Allyl 2,3,4,6-tetra-O-acetyl-α-D-glucopyranosyl-(1→4)-3,6-di-β-acetyl-2-deoxy-2-trichloroacetamido-α-D-glucopyranosyl-(1→2)-3,4-di-O-benzyl-α-L-rhamnopyranoside TMSOTf (8 μL, 0.04 mmol) was added to a solution of the trichloroacetimidate donor XX₁₆ (131 mg, 0.15 mmol) and known allyl 3,4-di-O-benzyl-α-L-rhamnopyranoside (ref. 76) (86 mg, 0.22 mmol) in toluene (4 mL) containing activated MS4 Å under argon. The mixture was stirred at −60° C. for 60 min and, when TLC monitoring indicated complete consumption of the donor, the reaction was quenched by addition of triethylamine. After filtration through a bed of celite and concentration under vacuum, flash chromatography (2:1 Cyclohexane-EtOAc) gave slightly contaminated trisaccharide XX₁₇ (Rf=0.2 in 2:1 Cyclohexane-EtOAc). ¹H NMR (CDCl₃, 400 MHz) δ (ppm): 7.41-7.28 (m, 10H, H arom.), 6.71 (d, 1H, J=8.7 Hz, NH), 5.91 (m, 1H, CH═), 5.45 (d, 1H, J₁₋₂=4.0 Hz, H1_{E}), 5.43-5.37 (m, 2H, H4_{D}, H3_{E}), 5.27 (m, 1H, ═CH₂), 5.19 (m, 1H, ═CH₂), 5.11-5.07 (m, 2H, H3_{D}, H4_{E}), 4.89 (dd, 1H, J₂₋₃=10.1 Hz, H2_{E}), 4.86 (d, 1H, OCH₂Ph), 4.80 (d, 1H, J₁₋₂=1.4 Hz, H1_{A}), 4.78 (d, 1H, OCH₂Ph), 4.62 (d, 1H, OCH₂Ph), 4.59 (d, 1H, OCH₂Ph), 4.56 (d, 1H, J₁₋₂=8.5 Hz, H1_{D}), 4.50 (m, 1H, H6a_{D}), 4.31-4.19 (m, 2H, H6b_{D}, H6a_{E}), 4.17-3.95 (m, 6H, 2 OCH₂, H2_{D}, H5_{D}, H5_{E}, H6b_{E}), 3.93 (dd, 1H, J₂₋₃=5.1 Hz, H2_{A}), 3.89 (dd, 1H, J₃₋₄=9.2 Hz, H3_{A}), 3.76-3.68 (m, 1H, H5_{A}), 3.38 (pt, 1H, J₄₋₅=9.2 Hz, H4_{A}), 2.17, 2.12, 2.10, 2.06, 2.05, 2.04 (6s, 18H, 6 COCH₃), 1.30 (d, 1H, J₅₋₆=5.1 Hz, H6_{A}); ¹³C NMR (CDCl₃, 100 MHz) δ (ppm): 171.4, 170.5, 170.3, 170.3, 169.4, 161.8 (7C, C═O), 138.5, 138.3 (2C, C quat. arom.), 133.8 (CH═), 128.9, 128.5, 128.4, 128.2, 127.7, 127.6 (10C, C arom.), 117.2 (═CH₂), 101.4 (C1_{D}), 98.2 (C1A), 95.5 (C1_{E}), 92.1 (CCl₃), 81.0 (C4_{A}), 80.0 (C3_{A}), 76.8 (C2_{A}), 75.6 (OCH₂Ph), 75.0 (C3_{D}), 73.6 (OCH₂Ph), 73.2 (C4_{D}), 70.0 (C2_{E}), 69.4 (C3_{E}), 68.5 (C5_{E}), 68.0 (3C, C5_{A}, C5_{D}, C4_{E}), 67.8 (OCH₂), 62.5 (C6_{D}), 61.5 (C6_{E}), 55.9 (C2_{D}), 22.5, 21.1, 21.0, 20.8, 20.7 (6C, COCH₃), 17.9 (C6_{A}). HRMS (ESI⁺) for C₄₉H₆₀Cl3NO₂₁Na ([M+Na]⁺, 1126.2621) m/z 1126.2623.

### EXAMPLE 6

### Chemical Synthesis of Potential Acceptor Building Blocks to Oligosaccharide Fragments of S. flexneri 1b and/or 1a O-Antigens

Allyl 2-acetamido-3-O-acetyl-4,6-O-benzylidene-2-deoxy-α-D-glucopyranoside (ref. 69) (XX₁₉). Benzaldehyde dimethylacetal (2.6 mL, 17.55 mmol) and CSA (2.72 g, 11.70 mmol) were added to a solution of compound XX₁ (1.53 g, 5.85 mmol) in acetonitrile (50 mL). After 45 min, the methanol formed during the reaction was removed under reduced pressure and another 2 mL of benzaldehyde dimethylacetal were added to the mixture. After 60 min, the reaction mixture was cooled to 0° C., neutralized by addition of triethylamine and concentrated to dryness to afford crude XX₁₈ (ref. 75) (2.03 g) as a white solid (Rf=0.6, 1:1 Cyclohexane-Acetone). This material was used directly without further purification.

Ac₂O (20 mL, 211.6 mmol) was added dropwise to a solution of crude XX₁₈ (2.03 g) in anhydrous pyridine (50 mL) and the resulting mixture was stirred for one night at room temperature. The reaction was then quenched at 0° C. by addition of methanol, and the mixture was evaporated to dryness. The residue was purified by flash-chromatography (Rf=0.2, 7:3 Cyclohexane-Acetone) to obtain XX₁₉ (2.27 g, 99% over 2 steps) as a white solid. ¹H NMR (CDCl₃, 400 MHz) S (ppm): 7.49-7.45 (m, 2H, H arom.), 7.41-7.34 (m, 3H, H arom.), 5.91 (m, 1H, CH═), 5.85 (d, 1H, J=9.6 Hz, NH), 5.55 (s, 1H, H7), 5.35 (dd overlapped, 1H, H3), 5.32 (m overlapped, 1H, ═CH₂), 5.27 (m, 1H, ═CH₂), 4.90 (d, 1H, J₁₋₂=3.7 Hz, H1), 4.37 (ddd, 1H, J₂₋₃=10.6 Hz, H2), 4.30 (dd, 1H, J₅₋₆ₐ=4.8 Hz, J_{6a-6b}=10.3 Hz, H6a), 4.22 (m, 1H, OCH₂), 4.02 (m, 1H, OCH₂), 3.95 (ddd, 1H, J₄₋₅=9.5 Hz, J_{5-6b}=9.8 Hz, H5), 3.80 (pt, 1H, H6b), 3.74 (pt, 1H, J₃₋₄=9.5 Hz, H4), 2.08, 1.97 (2s, 6H, COCH₃); ¹³C NMR (CDCl₃, 100 MHz) δ (ppm): 171.4, 170.0 (2C, C═O), 137.0, 133.2, 129.1, 128.2, 128.1, 126.2 (7C, CH═, C arom.), 118.3 (═CH₂), 101.6 (C7), 97.2 (C1), 79.1 (C4), 70.3 (C3), 68.9, 68.7 (2C, C6, OCH₂), 63.0 (C5), 52.6 (C2), 23.2, 20.9 (2C, COCH₃). HRMS (ESI⁺) for C₂₀H₂₅NO₇Na ([M+Na]⁺, 414.1529) m/z 414.1525.

Allyl 2-acetamido-3-O-acetyl-6-O-benzyl-2-deoxy-α-D-glucopyranoside (ref. 69) (XX₂₀). Trifluoroacetic acid (1.1 mL, 14.81 mmol) was slowly added to an ice-cold mixture of XX₁₉ (1.30 g, 3.29 mmol) and triethylsilane (2.6 mL, 16.45 mmol) in CH₂Cl₂ (35 mL). After stirring the mixture for 2 h at 0° C., then for 4.5 h at room temperature, the reaction was quenched by addition of Et₃N and concentrated. Purification of the residue by flash column chromatography on silica gel (7:3→3:2, Cyclohexane-Acetone) gave α-glycoside XX₂₀ (0.84 g, 65%) and the de-O-acetylated analogue XX_{20A} (ref. 77) (284 mg, 21%).

Compound XX₂₀ had Rf=0.2 (7:3 Cyclohexane-Acetone): ¹H NMR (CDCl₃, 400 MHz) δ (ppm): 7.38-7.28 (m, 5H, H arom.), 5.89 (m, 1H, CH═), 5.84 (d, 1H, J=9.6 Hz, NH), 5.29 (m, 1H, ═CH₂), 5.22 (m, 1H, ═CH₂), 5.12 (dd, 1H, J₂₋₃=10.8 Hz, J₃₋₄=8.6 Hz, H3), 4.86 (d, 1H, J₁₋₂=3.6 Hz, H1), 4.63 (d, 1H, OCH₂Ph), 4.57 (d, 1H, OCH₂Ph), 4.26 (ddd, 1H, H2), 4.19 (m, 1H, OCH₂), 4.00 (m, 1H, OCH₂), 3.85-3.76 (m, 3H, H4, H5, H6a), 3.73 (dd, 1H, J_{5-6b}=3.9 Hz, J_{6a-6b}=10.2 Hz, H6b), 2.09, 1.95 (2s, 6H, COCH₃); ¹³C NMR (CDCl₃, 100 MHz) δ (ppm): 172.2, 170.1 (2C, C═O), 137.7 (C quat. arom.), 133.4 (CH═), 128.5, 127.8, 127.6 (5C, C arom.), 118.0 (═CH₂), 96.5 (C1), 74.1 (C3), 73.7 (OCH₂Ph), 70.3 (C5*), 70.0 (C4*), 69.9 (C6), 68.4 (OCH₂), 51.8 (C2), 23.2, 21.0 (2C, COCH₃). HRMS (ESI⁺) for C₂₀H₂₇NO₇Na ([M+Na]⁺, 416.1685) m/z 416.1670.

Compound XX_{20A} had Rf=0.2 (1:1 Cyclohexane-Acetone). ¹H NMR (CDCl₃, 400 MHz) δ (ppm): 6.03 (d, 1H, J=9.0 Hz, NH), 5.92 (m, 1H, CH═), 5.30 (m, 1H, ═CH₂), 5.24 (m, 1H, ═CH₂), 4.84 (d, 1H, J₁₋₂=3.8 Hz, H1), 4.60 (d, 1H, OCH₂Ph), 4.57 (d, 1H, OCH₂Ph), 4.20 (m overlapped, 1H, OCH₂), 4.14 (ddd overlapped, 1H, H2), 4.00 (m, 1H, OCH₂), 3.82-3.71 (m, 4H, H3, H5, H6a, H6b), 3.60 (pt, 1H, J₃₋₄=J₄₋₅=8.8 Hz, H4), 1.97 (s, 3H, COCH₃); ¹³C NMR (CDCl₃, 100 MHz) δ (ppm): 172.5 (C═O), 138.1 (C quat. arom.), 133.4 (CH═), 128.4, 127.8, 127.7 (5C, C arom.), 118.1 (═CH₂), 96.5 (C1), 74.0 (C3), 73.6 (OCH₂Ph), 71.7 (C4), 70.6 (C5), 69.6 (C6), 68.3 (OCH₂), 53.3 (C2), 23.3 (COCH₃).

Phenyl 2,3,4,6-tetra-O-acetyl-1-thio(3D-glucopyranoside (ref. 78) (XX₂₁). Thiophenol (7.9 ml, 76.9 mmol) and boron trifluoride etherate (6.3 ml, 51.2 mmol) were successively added at 0° C. to a solution of β-D-glucose pentaacetate (10 g, 25.6 mmol) in CH₂Cl₂ (100 mL). After 4 h stirring at this temperature, the organic solution was washed with saturated aqueous NaHCO₃ and water, then dried (Na₂SO₄) and concentrated to give XX₂₁ as a white foam (10.28 g, 91%). Compound XX₂₁ had Rf=0.5 (4:1 Cyclohexane-EtOAc). ¹H NMR (CDCl₃, 400 MHz) δ (ppm): 7.53-7.51 (m, 2H, H arom.), 7.36-7.32 (m, 3H, H arom.), 5.25 (pt, 1H, J₂₋₃=J₃₋₄=9.4 Hz, H3), 5.06 (pt, 1H, J₄₋₅=9.4 Hz, H4), 5.00 (dd, 1H, J₁₋₂=10.1 Hz, H2), 4.73 (d, 1H, H1), 4.25 (dd, 1H, J₅₋₆ₐ=5.0 Hz, J_{6a-6b}=12.3 Hz, H6a), 4.20 (dd, 1H, J_{5-6b}=2.7 Hz, H6b), 3.75 (ddd, 1H, H5), 2.11, 2.04, 2.01 (4s, 12H, COCH₃); ¹³C NMR (CDCl₃, 100 MHz) δ (ppm): 170.9, 170.5, 169.7, 169.6 (4C, C═O), 133.5, 132.1, 129.3, 128.8 (6C, C arom.), 86.1 (C1), 76.2 (C5), 74.4 (C3), 70.4 (C2), 68.7 (C4), 62.6 (C6), 21.1, 21.0, 20.9 (4C, COCH₃).

Phenyl 2,3,4,6-tetra-O-benzyl-1-thio-β-D-glucopyranoside (ref. 79) (XX₂₃). Methanolic sodium methoxide (0.5 M solution, 20 mL, 10.0 mmol) was added to a solution of peracetylated XX₂₁ (10.27 g, 23.3 mmol) in methanol (50 mL). After 7 h at room temperature, the reaction was quenched with Dowex-H⁺ resin, filtered, and concentrated. The crude tetraol (ref. 80) XX₂₂ was dissolved in N,N-dimethylformamide (100 mL) and a 60% suspension of NaH in oil (5.71 g, 143 mmol), and benzyl bromide (28.3 mL, 238 mmol), were added at 0° C. The mixture was allowed to reach room temperature and was stirred overnight. Excess NaH was neutralized with MeOH. After concentration to dryness, the perbenzylated product XX₂₃ (10.94 g, 72% over 2 steps) was purified by flash chromatography (9:1 Cyclohexane-EtOAc). Compound XX₂₃ had Rf=0.5 (9:1 Cyclohexane-EtOAc). ¹H NMR (CDCl₃, 400 MHz) δ (ppm): 7.64-7.59 (m, 2H, H arom.), 7.43-7.21 (m, 23H, H arom.), 4.94-4.84 (m, 4H, OCH₂Ph), 4.76 (d, 1H, OCH₂Ph), 4.70 (d overlapped, 1H, H1), 4.65-4.56 (m, 3H, OCH₂Ph), 3.82 (dd, 1H, J₅₋₆ₐ=2.0 Hz, J_{6a-6b}=10.9 Hz, H6a), 3.76 (dd overlapped, 1H, H6b), 3.74 (pt overlapped, 1H, H3), 3.68 (pt, 1H, J₃₋₄=J₄₋₅=9.4 Hz, H4), 3.54 (dd, 1H, J₁₋₂=9.8 Hz, J₂₋₃=8.6 Hz, H2), 3.53 (ddd, 1H, H5); ¹³C NMR (CDCl₃, 100 MHz) δ (ppm): 138.8, 138.5, 134.3 (4C, C quat. arom.), 132.3, 129.3, 128.8, 128.7, 128.6, 128.3, 128.2, 128.1, 128.0, 127.9, 127.8 (20C, C arom.), 87.9 (C1), 87.2 (C3), 81.3 (C2), 79.5 (C5), 78.3 (C4), 76.2, 75.8, 75.4, 73.8 (4C, OCH₂Ph), 69.5 (C6). HRMS (ESI⁺) for C₄₀H₄₀O₅SNa ([M+Na]⁺, 655.2494) m/z 655.2475.

Allyl 2,3,4,6-tetra-O-benzyl-α-D-glucopyranosyl-(14)-2-acetamido-3-O-acetyl-6-O-benzyl-2-deoxy-α-D-glucopyranoside (XX₂₄). NIS (353 mg, 1.57 mmol) and TMSOTf (65 μL, 0.36 mmol) were successively added to a solution of thioglycoside donor XX₂₃ (917 mg, 1.45 mmol) and glycosyl acceptor XX₂₀ (475 mg, 1.21 mmol) in 5:2 Et₂O—CH₂Cl₂ (14 mL) containing activated MS4 Å under argon. The mixture was stirred at 0° C. for 60 min. When TLC monitoring indicated reaction completion (Rf=0.3, 1:1 Cyclohexane-EtOAc), triethylamine was added. After filtration through a bed of celite and concentration under vacuum, the residue was purified by flash chromatography (7:3 Cyclohexane-Acetone) to give slightly contaminated XX₂₄ (780 mg). ¹H NMR (CDCl₃, 400 MHz) δ (ppm): 7.38-7.13 (m, 25H, H arom.), 5.93 (m, 1H, CH═), 5.86 (d, 1H, J=9.7 Hz, NH), 5.44 (dd, 1H, J₂₋₃=10.7 Hz, J₃₋₄=9.1 Hz, H3_{D}), 5.32 (m, 1H, ═CH₂), 5.25 (m, 1H, ═CH₂), 5.17 (d, 1H, J₁₋₂=3.3 Hz, H1_{E}), 4.91 (d, 1H, OCH₂Ph), 4.90 (d, 1H, J₁₋₂=4.0 Hz, H1_{D}), 4.83 (d, 1H, OCH₂Ph), 4.80 (d, 1H, OCH₂Ph), 4.67 (s, 2H, OCH₂Ph), 4.62-4.53 (m, 3H, OCH₂Ph), 4.45 (d, 1H, OCH₂Ph), 4.37 (ddd, 1H, H2_{D}), 4.35 (d, 1H, OCH₂Ph), 4.20 (m, 1H, OCH₂), 4.17 (pt, 1H, J₄₋₅=9.4 Hz, H4_{D}), 4.05-3.97 (m, 2H, OCH₂, H6a_{D}), 3.90 (pt, 1H, J₃₋₄=9.3 Hz, H3_{E}), 3.89 (m overlapped, 1H, H5_{D}), 3.89 (ddd, 1H, J₄₋₅=9.9 Hz, H5_{E}), 3.70 (dd, 1H, J_{6a-6b}=10.8 Hz, H6b_{D}), 3.65 (dd, 1H, J₃₋₄=9.2 Hz, J₄₋₅=9.9 Hz, H4_{E}), 3.54 (dd, 1H, J₅₋₆ₐ=3.3 Hz, H6a_{E}), 3.52 (dd, 1H, J₂₋₃=9.7 Hz, H2_{E}), 3.44 (dd, 1H, J_{5-6b}=1.8 Hz, J_{6a-6b}=10.6 Hz, H6b_{E}), 1.99, 1.96 (2s, 6H, 2 COCH₃); ¹³C NMR (CDCl₃, 100 MHz) δ (ppm): 171.7, 169.8 (2C, C═O), 138.4, 138.2, 137.9, 137.5 (5C, C quat. arom.), 133.1 (CH═), 128.2, 128.1, 128.0, 127.9, 127.8, 127.6, 127.5, 127.4, 127.3, 127.2, 127.1, 127.0 (25C, C arom.), 117.6 (═CH₂), 97.0 (C1_{E}, J_{C-H}=170 Hz), 96.1 (C1_{D}), 81.3 (C3_{E}), 79.5 (C2_{E}), 77.2 (C4_{E}), 75.3, 74.6 (2C, OCH₂Ph), 73.1 (C3_{D}), 73.0, 72.9, 72.8 (3C, OCH₂Ph), 72.7 (C4_{D}), 70.8 (C5_{E}), 70.2 (C5_{D}), 68.1 (2C, OCH₂, C6_{D}), 67.9 (C6_{E}), 51.9 (C2_{D}), 22.9, 20.9 (2C, COCH₃).

Allyl 2,3,4,6-tetra-O-benzyl-α-D-glucopyranosyl-(1→4)-2-acetamido-6-O-benzyl-2-deoxy-α-D-glucopyranoside (XX₂₅). Methanolic sodium methoxide (0.5 M solution, 0.4 mL, 0.2 mmol) was added to a stirred solution of XX₂₄ (742 mg, 0.92 mmol) in anhydrous methanol (7.5 mL). The reaction mixture was stirred for 1 day at room temperature by which time all the starting material had been consumed (Rf=0.4, 1:1 Cyclohexane-EtOAc). Excess base was neutralized with Dowex-H⁺ resin. After removal of the resin by filtration, the filtrate was concentrated, and the residue was purified by silica gel chromatography, eluting with 1:1 Cyclohexane-EtOAc to obtain compound XX₂₅ as a colourless oil (555 mg, 69%); ¹H NMR (CDCl₃, 400 MHz) δ (ppm): 7.39-7.18 (m, 25H, H arom.), 5.93 (m, 1H, CH═), 5.87 (d, 1H, J=8.4 Hz, NH), 5.32 (m, 1H, ═CH₂), 5.24 (m, 1H, ═CH₂), 5.03 (d, 1H, J₁₋₂=3.5 Hz, H1_{E}), 5.00 (d, 1H, J₁₋₂=3.7 Hz, H1_{D}), 4.96-4.83 (m, 5H, OCH₂Ph), 4.77 (d, 1H, OCH₂Ph), 4.62-4.43 (m, 4H, OCH₂Ph), 4.25-4.18 (m, 2H, 1H OCH₂, H2_{D}), 4.05-3.99 (m, 2H, 1H OCH₂, H3_{E}), 3.96 (dd, 1H, J₂₋₃=10.7 Hz, J₃₋₄=8.6 Hz, H3_{D}), 3.88 (ddd, 1H, J₄₋₅=10.0 Hz, J₅₋₆ₐ=3.5 Hz, H5_{E}), 3.81-3.60 (m, 6H, H4_{D}, H5_{D}, H6a_{D}, H6b_{D}, H4_{E}, H6a_{E}), 3.59 (dd, 1H, J₂₋₃=10.0 Hz, H2_{E}), 3.50 (dd, 1H, J_{5-6b}=1.7 Hz, J_{6a-6b}=10.6 Hz, H6b_{E}), 2.06 (s, 3H, COCH₃); ¹³C NMR (CDCl₃, 100 MHz) δ (ppm): 170.5 (C═O), 138.6, 138.5, 138.2, 137.9, 137.0 (7C, C quat. arom.), 133.8 (CH═), 128.7, 128.6, 128.5, 128.4, 128.3, 127.9, 127.8 (25C, C arom.), 117.6 (═CH₂), 100.6 (C1_{E}, J_{C-H}=172 Hz), 96.5 (C1_{D}), 82.2 (C3_{E}), 82.0 (C4_{D}), 79.6 (C2_{E}), 77.8 (C4_{E}), 75.7, 75.0, 74.1, 73.5, 73.2 (5C, OCH₂Ph), 72.0 (C3_{D}), 71.5 (C5_{E}), 70.2 (C5_{D}), 69.0 (C6_{D}), 68.5 (C6_{E}), 68.4 (OCH₂), 53.3 (C2_{D}), 23.4 (COCH₃). HRMS (ESI⁺) for C₅₂H₅₉NO₁₁Na ([M+Na]⁺, 896.3986) m/z 896.4020.

### EXAMPLE 7

### Synthesis and Characterization of α-D-glucopyranosyl-(1→4)-N-acetyl-D-glucosamine (P2)

Glucosylation of N-acetyl-D-glucosamine (acceptor) α-D-glucopyranosyl-(1→4)-N-acetyl-D-glucosamine (P2) is the main acceptor reaction product obtained by action of 1G5A amylosucrase (AS) (Recombinant form of Neisseria polysaccliarea amylosucrase EC2.4.1.4 (GH13)) using sucrose as donor and D-GlcpNAc as acceptor in a molar ratio of 1.

### 7.1. Materials

Recombinant enzyme were produced in E. coli as reported elsewhere. Purified AS, conserved at −20° C. or −80° C., served for enzymatic reaction.

N-acetyl-D-glucosamine was purchased from Sigma-Aldrich.

### 7.2. Acceptor Reaction Assay

The glucosylation reaction was performed in the enzyme optimal buffer: in Tris-HCl (50 mM, pH=7.5) for AS assay. The reaction mixture was carried out at 30° C. with sucrose and acceptor in equimolar ratio (146 mM). AS were used at 1 U/mL. Activity one unit is defined as the amount of enzyme that catalyzes the formation of 1 μmol of fructose/min at 30° C., in enzyme buffer and sucrose at a concentration of 146 mM. The reaction was stopped by heating at 95° C. for 5 min. The final mixture was centrifuged at 18 000 g for 10 min and filtered on a 0.22 μm membrane before HPLC analysis.

### 7.3. Glucosyl Acceptor Production

In order to characterize glucosylated products of N-acetyl-D-glucosamine, acceptor reactions were conducted at preparative scale.

P2 from N-acetyl-D-glucosamine glucosylation by purified AS (1 U/mL) was produced in 100 mL mixture reaction (292 mM in sucrose, 730 mM in acceptor).

After a 24 h reaction time at 30° C., the media were centrifugated at 4800 rpm, for 20 min at 4° C. to remove proteins and filtered for a better clarification. The purification of the glucosylated products was performed on a preparative octadecyl reverse-phase chromatography column (C18 column) (Bischoff Chromatography). Ultra pure water was used as eluent at a constant flow rate of 50 mL/min. Glucosyl detection was carried out with a refractometer, and each peak was collected separately, concentrated and reinjected into an analytical HPLC system to check the purity of the compounds.

### 7.4. Analytical Methods

7.4.a. High Performance Liquid Chromatography (HPLC)

HPLC analysis device consisted in a Dionex P 680 series pump, a Shodex RI 101 series refractometer, a Dionex UVD 340 UV/Vis detector and an autosampler HTC PAL. Five columns were employed to separate the acceptor reaction products and to determine the acceptor conversion rate and product yields (i) a Biorad HPLC Carbohydrate Analysis columns: AMINEX HPX-87C at 80° C. (elution with ultra-pure water at 0.6 mL/min) (ii) HPX-87K columns (300×7.8 mm) at 65° C. (elution with ultra-pure water at 0.6 mL/min) (iii) C18 column Bischoff Prontosil Eurobond, 5 μm (elution with ultra pure water at room temperature and 1 mL/min) (iv) C30: Bischoff Prontosil Eurobond, 5 μm, 250×4.0 mm (elution with ultra pure water at room temperature and 1 mL/min) (v) C18RP: Sinergi Fusion RP Phenomenex, 4 μm, 250×4.6 mm (elution with ultra pure water at room temperature and 1 mL/min)

7.4.b. High Resolution Mass Spectrometry (HRMS) and Nuclear Magnetic Resonance (NMR)

Accurate mass determination was carried out using an Autospec mass spectrometer arranged in an EBE geometry (Micromass, Manchester, UK). The instrument was operated at 8 kV accelerating voltage in positive mode. The caesium gun was set to 35 keV energy and 1 μL, of sample was mixed in the tip of the probe with a glycerol or dithiothreitol/dithioerythritol matrix.

NMR analyses: ¹H (400.130 MHz), ¹³C (100.612 MHz), HSQC and HMBC were registered on a Bruker-ARX 400 spectrometer equipped with an ultrashim system. Samples were dissolved in deuterium oxide at c.a. 80 g/L and experiments were performed at 300K.

### EXAMPLE 8

### Engineering Double Mutant Amylosucrases for the Synthesis the α-D-glucopyranosyl-(1→4)—N-acetyl-α-D-glucopyranosaminyl disaccharide

### 1) Materials and Methods

### Plasmids, Bacterial Strains and Chemicals

Plasmid pGST-AS (see Example 1-1) was used for the construction of the AS double-mutant libraries.

Fusion DNA-polymerase was purchased from Finnzymes (Espoo, Finland), and DpnI restriction enzyme from New England Biolabs (Beverly, Mass., USA).

Oligonucleotides were synthesised by Eurogenetec (Liege, Belgium). DNA extraction (QIASpin) and purification (QIAQuick) columns were purchased from Qiagen (Chatsworth, Calif.).

E. coli TOP 10 electrocompetent cells (Invitrogen, Carlsbad, USA) were used as host for the plasmid library transformation and gene expression. DNA sequencing was performed by Cogenics (Meylan, France). All positive clones for D-GlcpNAc glucosylation were sequenced on the mutated region (˜600 bp) using the primer pGEX_int: CCAACGAACACGAATGGGC (SEQ ID NO: 28).

Ampicillin (Amp), lysozyme, and isopropyl-β-D-thiogalactopyranoside (IPTG) were purchased from Euromedex (Souffelweyersheim, France); Bromothymol Blue sodium salt, sucrose and N-acetyl-D-glucosamine (D-GlcpNAc) from Sigma-Aldrich (Saint-Louis, Mo., USA).

Reference disaccharides α-D-Glcp-(1→4)-D-GlcpNAc and α-D-Glcp-(1-6)-D-GlcpNAc were enzymatically synthesized and characterized (see Example 7).

### Construction of Libraries Containing Two Vicinal Mutations (Libraries 1-3)

Site-saturation mutagenesis, focused on the vicinal positions (I228-F229, A289-F290, I330-V331) of AS +1 acceptor subsite was carried out using pGST-AS G537D as vector template. It was checked that this mutation had no impact on the native enzyme catalytic properties

Three partial overlapping primer pairs surrounding double codons were designed. Each of these codons was replaced in the primers with degenerate NNS or NNW sequence, where N=A, C, G or T; S=C or G and W=A or T (Table V below). Such degenerate primers were designed to generate 32 codons encoding the 20 possible amino acids.

TABLE V
Degenerate primers used for the construction of
librairies (I228-F229, A289-F290, I330-V331)

| SEQ ID | Primer | Nucleotide sequence |
|---|---|---|
| NO: | name | 5′ to 3′ |
| 29 | 228-229 for | CTG CGC GAA NNS NNS CCC GAC CAG CAC CCG GGC G |
| 30 | 228-229 rev | CTG GTC GGG WNN WNN TTC GCG CAG GGT GCG GTC G |
| 31 | 289-290 for | GCG GTT NNS NNW ATT TGG AAA CAA ATG GGG ACA AGC TGC G |
| 32 | 289-290 rev | CCA AAT SNN WNN AAC CGC ATC CAT ACG CAG GAT GTC AAC GCC |
| 33 | 330-31 for | TCC GAA GCC NNS NNS CAC CCC GAC CAA GTC GTC C |
| 34 | 330-31 rev | GGG GTG WNN WNN GGC TTC GGA TTT GAA GAA CAC GGC |

PCR amplification was carried out on the whole plasmid with Phusion DNA-polymerase (1 U) for 30 cycles (98° C., 10 s; 75° C., 20 s; 57° C., 15 s; 72° C., 5 min). The DNA was digested with DpnI to eliminate methylated parental template and purified using Qiaquick spin column, following manufacturer's recommendations. E. coli TOP10 was transformed by electroporation with 44 of each plasmid library using standard procedures.

### Construction of Library Containing Two Distant Mutations (Library 4)

pGST-AS G537D was also used as vector template for library (I228-F290) construction. First, for each targeted position, the 2×19 complementary primers previously constructed by site-directed mutagenesis (see Example 1-1) were pooled in equimolar ratio, that thus formed two pairs of degenerate primers, for positions 228 and 290, respectively. Then, it was proceeded two PCR amplification steps using the same PCR method, DNA treatment and cell transformation as described above. The first step consisted in generating individually two mono-mutants libraries by saturation mutagenesis. After plasmid library extraction, a second PCR step was carried out to introduce the second mutation.

### HPLC Screening of Variants Able to Glucosylate D-GlcpNAc

The method for the expression of the mutant libraries is described in Example 1-1.

Acceptor reaction products were analyzed by HPLC analysis using a C18-AQ column (Bischoff C18, 125×4 mm, 3 μm) kept at room temperature and eluted with 0.6 mL/min of ultra-pure water to detect α-D-Glcp-(1→4)-α-D-GlcpNAc formation (analysis time: 7 min). To evaluate the efficiency of the glucosylation reaction, it was carried out complementary HPLC analyses with a Biorad HPX-87K Carbohydrate Analysis column (maintained at 65° C., and eluted at a flow rate of 0.6 mL/min with ultra-pure water). By this way, it was possible to measure sucrose consumption by RI detection and concomitant α-D-Glcp-(1-4)-α-D-GlcpNAc formation by UV220 nm and determine mutant ability to synthesize the desired disaccharide α-D-Glcp-(1→4)-α-D-GlcpNAc (i.e., % Glucosyl units transferred onto acceptor derivatives=[Glucosyl units transferred onto acceptor derivatives]/[Glucosyl units transferred from initial sucrose]×100)

### Production, Purification and Characterization of the Selected Variants: A289P-F290C, A289P-F2901 and A289P-F290L.

The double mutants A289P-F290C, A289P-F290I and A289P-F290L were produced and purified, as previously described in Example 1-1. The protein content was determined by the Nanodrop ND-1000 spectrophotometer. These mutants were tested on sucrose alone or supplemented with α-D-GlcpNAc-OAll acceptor, and compared to wtAS and F290K. Assays were performed at 30° C. in 50 mM Tris-HCl buffer, pH=7.0.

### Standard Activity Determination

One unit of amylosucrase activity corresponds to the amount of enzyme that catalyzes the release of 1 μmol of reducing sugars per minute in the assay conditions. When using sucrose as sole substrate, specific activity was determined using 250 mM sucrose. In the presence of both sucrose and α-D-GlcpNAc-OAll acceptor, specific activity was determined using 250 mM sucrose and 250 mM α-D-GlcpNAc-OAll. The concentration of reducing sugars was determined using the dinitrosalicylic method (ref. 35) and fructose as standard.

Comparison of Products Synthesized by Wild-Type (wtAS) and AS Variants

Reactions were performed in the presence of 250 mM sucrose alone or supplemented with 250 mM α-D-GlcpNAc-OAll acceptor. The purified wtAS or mutated AS were employed at 1 U/mL. The reactions were stopped after 24 h by heating at 95° C. for 5 min. The soluble part of the reaction mixture was submitted to HPAEC (high-performance anion-exchange chromatography with pulsed amperometric detection). To quantify the concentration of monosaccharides and disaccharides, the soluble fraction was diluted in water and separated on a 4*250 mm Dionex Carbo-pack PA100 column. A gradient of sodium acetate (from 6 to 300 mM in 28 min) in 150 mM NaOH was applied at 1 ml/min flow rate. Detection was performed using a Dionex ED40 module with a gold working electrode and an Ag/AgCl pH reference. α-D-GlcpNAc-OAll, and their derivatives, are not oxidable products and thus are not detectable by HPAEC. Therefore, α-D-GlcpNAc-OAll and their glucosylation products, were quantified by HPLC with a Biorad HPX-87K (see above). Concentration of sucrose, glucose, fructose, turanose, trehalulose and maltose was determined by HPAEC.

In parallel, the reaction mixture containing soluble and insoluble malto-oligosaccharides was solubilized in 1 M aq KOH at a final total sugar concentration of 10 g/L and analysed by HPAEC using a Dionex Carbo-Pack PA100 column at 30° C. Mobile phase (150 mM aq NaOH) was set at 1 mL/min flow rate with a sodium acetate gradient (6 to 500 mM over 120 min).

### Determination of Kinetic Parameters Towards Sucrose Donor

Enzyme assays were carried out in a total volume of 2 mL containing pure enzyme (0.073 mg, 0.153 mg and 0.092 mg when using A289P-F290C, A289P-F2901 and A289P-F290L, respectively).

Kinetic studies of AS variants were performed in the presence of sucrose (0-500 mM). At regular time intervals (2-5 min), aliquots (200 μL) were removed, heated (95° C., 2 min) and centrifuged (18000 g, 5 min) All the samples were filtered on a 0.22 μm membrane and analyzed using HPLC material previously described. The initial rate of sucrose consumed, corresponding to the initial rate of fructose released, was expressed in mole of fructose released per minute and per gram of enzyme. The kinetic parameters k_{cat} and Kₘ were calculated using Eadie-Hofstee plot.

### Determination of Kinetic Parameters Towards α-D-GlcpNAc-OAll Acceptor

Enzyme assays were carried out in a total volume of 2 mL containing pure enzyme (0.037 mg, 0.076 mg and 0.046 mg when using A289P-F290C, A289P-F2901 and A289P-F290L, respectively).

Catalytic efficiency (k_{cat}/Kₘ) of AS variants towards α-D-GlcpNAc-OAll acceptor was determined using sucrose (250 mM) and acceptor α-D-GlcpNAc-OAll as variable substrate (0-250 mM) and following the same protocol as described above.

The kcat/Km (α-D-GlcpNAc-OAll) value was calculated from the initial rate of formation of desired disaccharide (α-D-Glcp-(1→4)-α-D-GlcpNAc-OAll), corresponding to the initial rate of α-D-GlcpNAc-OAll consumed. As saturation was not achieved with the mutants, efficiency was calculated by linear regression analysis of the velocity versus substrate concentration plot.

### 2) Results

### Library Construction and Pre-Screening of Sucrose-Utilizing Variants

It was showed in Example 1 that positions 228 and 290 are key positions for altering AS selectivity towards D-GlcpNAc. However, given the spatial vicinity of these two sites with positions 229, 289 and 330, and the influence they might have on each other, it was constructed libraries focused on all 5 positions 228, 289, 290, 330, and 331.

To enlarge the exploration of subsite +1 sequence space, it was generated four libraries of double-mutants on adjacent or distant positions before attempting a full recombination involving all positions. Indeed, such double-mutants are not easily accessible from error-prone PCR because no polymerase is able to generate successive errors on a sequence and also due to the genetic code degenerescence. Libraries 1, 2 and 3 targeting positions 228-229, 289-290, 330-331, respectively, were constructed by PCR using a set of degenerate primers designed to generate the 20 possible amino acids (see FIG. 12). In this way, 400 (20×20) possible double mutations were encoded. To ensure a good representation of all the variants, it was estimated that 3000 recombinant clones had to be screened for each library. The strategy employed for the construction of library 4, corresponding to the combination of positions 228 and 290, was slightly different. PCR was carried out using successively two sets of primers, each of them encoding for the 19 possible amino acids. All possible amino acid changes are considered possible in this library.

The libraries were then screened to determine both their size and the ratio of active clones using the pH indicator screen (see the method described in Example 1), as shown in Table VI below.

TABLE VI
Number of clones and active clones obtained for the four librairies

| | Library 1 | Library 2 | Library 3 | Library 4 |
|---|---|---|---|---|
| | 228-229 | 289-290 | 330-331 | 228-290 |
| Number of clones | ~8000 | ~2000 | ~3200 | ~8000 |
| Number of active | 180 (2.3%) | 576 (29%) | 576 (18%) | 384 (4.8%) |
| clones isolated (%) | | | | |
| Number of active | 96 | 96 | 96 | 96 |
| clones (yellow) | | | | |
| Number of | 84 | 480 | 480 | 288 |
| moderatly active | | | | |
| clones (green) | | | | |

A total of 20 000 recombinant clones were screened. Over 3000 recombinant clones were obtained for each library except for library 2, for which only 2000 clones were generated. Altogether, 1716 clones were found to be able to use sucrose as a glucosyl donor. Depending on the library, the ratio of active clones was comprised between 2 and 29%. Based on the color change of BBT, 384 clones induced a strong pH change whereas 1332 were found to be moderately active.

### Isolation of Improved Mutants for α-D-Glcp-(1→4)-α-D-GlcpNAc Synthesis

All clones producing sucrose-utilizing variants were then picked and cultured in 96-well microplates. Following cell lysis, the acceptor reaction in the presence of D-GlcpNAc was then carried out in microplate format. HLPC analysis time for each clone was reduced to 7 min for detection of α-D-Glcp-(1→4)-α-D-GlcpNAc. When disaccharide formation was detected, complementary analyses were performed to determine sucrose consumption and disaccharide formation with more accuracy in order to calculate the glucosylation yield.

### Improved Mutants for α-D-Glcp-(1→4)-D-GlcpNAc Synthesis

Of the 1716 tested mutants, 30 were able to catalyze α-D-Glcp-(1→4)-D-GlcpNAc synthesis with a glucosylation rate higher than 15% (see Table VII below).

TABLE VII
Improved mutants for α-D-Glcp-(1→4)-α-D-GlcpNAc (ED) synthesis

| | | Single-Mutant | | | |
|---|---|---|---|---|---|
| | | (SM) or | | | Glusosylation |
| | Library | Double-Mutant | Amino acid | Mutation(s) | Yield to |
| Mutants | number | (DM) | Mutation(s) | (codon) | synthesize ED |
| wtAS | | | | | 2% |
| DM2_P1_B8 | 2 | DM | A289P-F290C | CCG, TGT | 100% |
| DM2_P1_B6 | 2 | DM | A289P-F290C | CCG, TGT | 100% |
| DM2_P4_F7 | 2 | DM | A289P-F290I | CCC, ATT | 100% |
| DM2_P4_H5 | 2 | DM | A289P-F290L | CCC, TTA | 100% |
| DM4_P1_H10 | 4 | SM | F290V | GTT | 100% |
| DM4_P1_H11 | 4 | SM | F290R | CGT | 100% |
| DM4_P1_G7 | 4 | DM | I228V-F290V | GTC, GTT | 90% |
| DM4_P1_C7 | 4 | DM | I228V-F290K | GTC, AAA | 89% |
| DM4_P1_C1 | 4 | DM | I228A-F290H | GCC, CAT | 88% |
| DM1_P1_C1 | 1 | SM | F229V | GTG | 88% |
| DM1_P1_C12 | 1 | SM | F229M | ATG | 79% |
| DM1_P1_D11 | 1 | DM | I228C-F229L | TGC, CTC | 78% |
| DM4_P3_B8 | 4 | DM | I228T-F290K | ACC, AAA | 72% |
| DM4_P1_C10 | 4 | DM | I228V-F290H | GTC, CAT | 61% |
| DM4_P1_A6 | 4 | DM | I228T-F290H | ACC, CAT | 56% |
| DM1_P2_F7 | 1 | DM | I228V-F229L | GTG, TTG | 56% |
| DM4_P2_A2 | 4 | SM | F290L | CTT | 55% |
| DM4_P2_H10 | 4 | SM | F290G | GGT | 53% |
| DM4_P2_C2 | 4 | DM | I228K-F290W | AAG, TGG | 50% |
| DM1_P2_C8 | 1 | SM | F229L | CTC | 49% |
| DM4_P2_B10 | 4 | DM | I228V-F290V | GTC, GTT | 47% |
| DM4_P2_G4 | 4 | DM | I228K-F290L | AAG, CTT | 47% |
| DM4_P3_C8 | 4 | SM | F290H | CAT | 46% |
| DM4_P2_H4 | 4 | SM | F290K | AAA | 42% |
| DM4_P2_D12 | 4 | SM | F290K | AAA | 41% |
| DM2_P1_G10 | 2 | DM | A289T-F290H | ACG, CAT | 40% |
| DM2_P3_C3 | 2 | DM | A289H-F290S | CAC, TCT | 40% |
| DM1_P1_H2 | 1 | DM | I228L-F229L | TTG, CTC | 35% |
| DM1_P2_F2 | 1 | DM | I228V-F229M | GTG, ATG | 29% |
| DM1_P2_C3 | 1 | DM | I228M-F229M | ATG, ATG | 28% |
| DM4_P3_G9 | 4 | DM | I228V-F290R | GTC, CGT | 25% |
| DM4_P3_F7 | 4 | DM | I228P-F290C | CCC, TGT | 22% |
| DM4_P2_G9 | 4 | SM | F290L | CTT | 20% |
| DM4_P4_B12 | 4 | SM | F290I | ATT | 16% |

Among these positive mutants, it was distinguished 20 double-mutants, 6 single-mutants previously identified (see Example 1) and 4 new single-mutants (F290R, F229L, F229M and F229V). Out of these 30 mutants, 4 were identified twice (A289P-F290C, I228V-F290V, F290K and F290L). Most active double-mutants (13 out of 20) show a mutation at position 290, a position that was previously identified as critical for D-GlcpNAc recognition. The glucosylation rates determined for F290L and I228V F290V mutants differed among the two identified clones from 20 to 55% and from 47% to 90%, respectively. These variations reflect the poor reproducibility of the glucosylation assay in microtiter plates. Microtiter experiments combined with HPLC analysis are designed for high-throughput screening. Consequently, to evaluate the impact of the mutations on the catalytic efficiency for sucrose and α-D-Glcp-(1→4)-α-D-GlcpNAc-OAll formation, the most improved mutants, particularly the double-mutants A289P-F290C, A289P-F290I and A289P-F290L were further characterized and compared with F290K, which was the more appropriated mono-mutant for α-D-GlcpNAc-OAll glucosylation.

### Characterization of A289P-F290C, A289P-F2901 and A289P-F290L Mutants

Mutants A289P-F290C, A289P-F290I and A289P-F290L were produced and purified to homogeneity to determine their kinetic parameters and their product reaction profile. They were both compared to that obtained with F290K and wtAS.

In the presence of sucrose alone (250 mM), all 3 double-mutants kept the ability to synthesize maltooligosaccharides (up to DP 20), similarly to F290K (see FIG. 13A). In addition, all three double mutants produced an increased amount of sucrose isomers (trehalulose and mostly turanose) compared to wtAS (see FIG. 13B).

When α-D-GlcpNAc-OAll was added, the three double mutants displayed a very high specificity for the acceptor, as observed for F290K. Indeed, maltooligosaccharide production was fully suppressed in favour of α-D-GlcpNAc-OAll glucosylation (see FIG. 14A). The quasi-totality of the sucrose glucosyl residues was transferred onto α-D-GlcpNAc-OAll, converting α-D-GlcpNAc-OAll into α-D-Glcp-(1→4)-α-D-GlcpNAc-OAll (46% to 64%) and a di-glucosylated form (α-D-Glcp-(1→4)-α-D-Glcp-(1→4)-α-D-GlcpNAc-OAll) (13% to 24%) (see FIG. 14B).

To compare the improved catalytic properties of the double-mutants, it was first investigated the kinetic parameters of these mutants towards sucrose donor. Upon varying the sucrose concentration (0-250 mM), all three double-mutants showed standard saturation kinetic behavior (Table VIII).

TABLE VIII
Comparison of the kinetic parameters of wtAS, F290K and improved
double-mutants (A289P-F290C, A289P-F290I and A289P-F290L)
for sucrose donor.

| | | | | V_{250mM}^{b} | behaviour |
|---|---|---|---|---|---|
| | k_{cat} | K_{M} | k_{cat}/K_{M} | (μmol/ | (at high |
| | (s−1) | (mM) | (s−1 mM−1) | min/g) | concentration) |
| wtAS^{a} | 1.3 | 50.2 | 0.0261 | 900 | saturation |
| F290K | n.d. | n.d. | 0.0006 | 110 | linear |
| A289P-F290C | 2.8 | 18.9 | 0.1502 | 2186 | saturation |
| A289P-F290I | 1.1 | 17.2 | 0.0655 | 946 | saturation |
| A289P-F290L | 2.1 | 10.1 | 0.2094 | 1717 | saturation |

n.d.: not determined;
^{a}Data from ref. 83;
^{b}Initial rate of sucrose consumption was determined at a concentration of 250 mM for sucrose.

In contrast, no kinetic saturation was achieved with F290K, which displayed a poor affinity for sucrose. The kcat/Km of the double-mutants was improved 100 to 350-fold relative to that of F290K. In comparison to wtAS, the catalytic efficiency of the 3 screened mutants toward sucrose was increased by 2.5 to 8-fold. For all double-mutants, the Km and the kcat turnover values were improved (Table VIII above).

To investigate the effect of the double mutations on the acceptor specificity of the amylosucrase, kinetic parameters were determined for various acceptor concentrations using a fixed donor sugar concentration (250 mM). Results are shown in Table IX below.

TABLE IX
Comparison of the kinetic parameters of wtAS, F290K and
improved double-mutants (A289P-F290C, A289P-F290I and
A289P-F290L) for α-D-GlcpNAc-OAll acceptor with
sucrose fixed at 250 mM.

| | k_{cat}/K_{M} | V_{250mM}^{a} |
|---|---|---|
| | (s−1 mM−1) | (μmol/min/g) |
| wtAS | 0.002 | 290 |
| F290K^{a} | 0.265 | 6690 |
| A289P-F290C | 0.690 | 50 000 |
| A289P-F290I | 0.700 | 51 400 |
| A289P-F290L | 0.790 | 67 300 |

^{b}Initial rate of acceptor consumption was determined at a concentration of 250 mM for both sucrose and α-D-GlcpNAc-OAll.

The catalytic efficiency of the 3 double-mutants towards α-D-GlcpNAc-OAll was increased by up to a remarkable 395-fold compared to wtAS and 3-fold compared to variant F290K.

### REFERENCES

1. Gabius, H. J. et al., Chembiochem 5, 740-764 (2004).
2. Ruffing, A. & Chen, R. R. Microbial cell factories 5, 25 (2006).
3. Persidis, A. Nature biotechnology 15, 479-480 (1997).
4. Seeberger, P. H. & Werz, D. B. Nature 446, 1046-1051 (2007).
5. Kilcoyne, M. & Joshi, L. Cardiovascular & hematological agents in medicinal chemistry 5, 186-197 (2007).
6. Lee, J. C. et al., Nature protocols 1, 3143-3152 (2006).
7. Plante, O. J. et al., Science (New York, N.Y.) 291, 1523-1527 (2001).
8. Wang, C. C. et al., Nature 446, 896-899 (2007).
9. Joe, M. et al., Journal of the American Chemical Society 129, 9885-9901 (2007).
10. Drouillard, S. et al., Angewandte Chemie (International ed) 45, 1778-1780 (2006).
11. Lindberg, A. A., Vaccine 17 Suppl 2, S28-36 (1999).
12. Ada, G. & Isaacs, Clin Microbiol Infect 9, 79-85 (2003).
13. Pirofski, L. A., Trends in microbiology 9, 445-451 (2001).
14. Nyame, A. K. et al., Archives of biochemistry and biophysics 426, 182-200 (2004).
15. Ragupathi, G., Cancer Immunol Immunother 43, 152-157 (1996).
16. Pozsgay, V., Advances in carbohydrate chemistry and biochemistry 56, 153-199 (2000).
17. Verez-Bencomo, V. et al., Science (New York, N.Y.) 305, 522-525 (2004).
18. Thiem, J., FEMS microbiology reviews 16, 193-211 (1995).
19. Koeller, K. M. & Wong, C. H., Nature 409, 232-240 (2001).
20. Yan, F. et al., Carbohydrate research 328, 3-16 (2000).
21. Hanson, S. et al., Trends Biochem Sci 29, 656-663 (2004).
22. Crout, D. H. & Vic, G., Current opinion in chemical biology 2, 98-111 (1998).
23. Schroven, A. et al., Chemistry (2007).
24. Schmidt, D. et al., J Org Chem 65, 8518-8526 (2000).
25. Yoon, J. H. & Ajisaka, K., Carbohydrate research 292, 153-163 (1996).
26. Hancock, S. M. et al., Current opinion in chemical biology 10, 509-519 (2006).
27. Jank, T. et al., The Journal of biological chemistry 280, 37833-37838 (2005).
28. Zhang, J. H. et al., Proceedings of the National Academy of Sciences of the United States of America 94, 4504-4509 (1997).
29. Parikh, M. R. & Matsumura, I., Journal of molecular biology 352, 621-628 (2005).
30. Osanjo, G. et al., Biochemistry 46, 1022-1033 (2007).
31. Hancock, S. M. et al., Chembiochem 6, 866-875 (2005).
32. Kim, Y. W. et al., The Journal of biological chemistry 279, 42787-42793 (2004).
33. Honda, Y. & Kitaoka, M., The Journal of biological chemistry 281, 1426-1431 (2006).
34. Niggemann, J. et al., Bioorg Med Chem 6, 1605-1612 (1998).
35. Eichler, E. et al., Carbohydrate research 319, 1-16 (1999).
36. Mehta, S. et al., Org Lett 2, 751-753 (2000).
37. Yan, F. et al., J Org Chem 68, 2426-2431 (2003).
38. Yan, F. et al., Org Lett 3, 3265-3268 (2001).
39. Kotloff, K. L. et al., Bulletin of the World Health Organization 77, 651-666 (1999).
40. Levine, M. M. et al., Nature reviews 5, 540-553 (2007).
41. Lindberg, A. A. et al., Reviews of infectious diseases 13 Suppl 4, S279-284 (1991).
42. Jennison, A. V. & Venna, N. K., FEMS microbiology reviews 28, 43-58 (2004).
43. Phalipon, A. et al., J Immunol 176, 1686-1694 (2006).
44. Simmons, D. A., Biochemical Society transactions 18, 1271-1272 (1990).
45. De Montalk, G. P. et al., Journal of bacteriology 181, 375-381 (1999).
46. Albenne, C. et al., The Journal of biological chemistry 279, 726-734 (2004).
47. Yoon, S. H. & Robyt, J. F., Carbohydrate research 337, 2427-2435 (2002).
48. Cote, G. L. et al., Carbohydrate research 340, 257-262 (2005).
49. Richard, G. et al., Carbohydrate research 340, 395-401 (2005).
50. Demuth, K. et al., Carbohydrate research 337, 1811-1820 (2002).
51. Coutinho, P. M. & Henrissat, B., in Recent Advances in Carbohydrate Bioengineering. (eds. H. J. Gilbert, G. Davies, B. Henrissat & B. Svensson) 3-12 (The Royal Society of Chemistry, Cambridge; 1999).
53. Skov, L. K. et al., Acta Crystallogr D Biol Crystallogr 56, 203-205 (2000).
54. Mirza, O. et al., Biochemistry 40, 9032-9039 (2001).
55. Skov, L. K. et al., The Journal of biological chemistry 277, 47741-47747 (2002).
56. Bradford, M. M., Analytical biochemistry 72, 248-254 (1976).
57. Sumner, J. & Howell, S. A., J. Biol. Chem. 108, 51-54 (1935).
58. Clement, M. J. et al., The Journal of biological chemistry 278, 47928-47936 (2003).
59. West, N. P. et al., Science 307, 1313-1317 (2005).
60. Allison, G. E. & Verma, N. K., Trends in microbiology 8, 17-23 (2000).
61. Ovodov, Y. S., Biochemistry 71, 937-954 (2006).
62. Davies, G. & Henrissat, B., Structure 3, 853-9 (1995).
63. Henrissat, B., Biochem J. 280, 309-16 (1991).
64. Henrissat, B. & Bairoch, A., Biochem J 293, 781-8 (1993).
65. Stam, M. R. et al., Protein Eng Des Sel. 19, 555-62 (2006).
66. Lemanski, G. & Ziegler, T., Eur. J. Org. Chem. 2618-2630 (2006).
67. Rye C. S. & Withers S. G., Curr Opin Chem Biol 4, 573-80 (2000).
68. Skov, L. et al., J Biol Chem 276, 25273-8 (2001).
69. Ghosh M. et al., JOC 65, 8387-8390 (2000).
70. Cai, Y. et al., Org. Lett. 7, 4021-4024 (2005).
71. McGeary, R. P. et al., J. Org. Chem. 66, 5102-5105 (2001).
72. Blatter, G. & Beau, J. M., Carbohydr. Res. 260, 189-202 (1994).
73. Poszgay, V., J. Org. Chem. 63, 5983-5999 (1998).
74. Palomino, J. et al., J. Carbohydr. Chem. 15, 137-146 (1996).
75. Feng, F. et al., Org. Biomol. Chem. 2, 1617-1623 (2004).
76. Westerduin, P. et al., Carbohydr. Res. 180, 195-206 (1988).
77. Cai, J. et al., Carbohydr. Res. 300, 109-118 (1997).
78. Cheng, M. S. et al., J. Org. Chem. 68, 3658-3662 (2003).
79. Damager, I. et al., Carbohydr. Res. 320, 19-30 (1999).
80. Blom, P. et al., J. Org. Chem. 70, 10109-10112 (2005).
81. Emond, S. et al., J Biomol Screen. 12, 715-23 (2007).
82. Banks, W. et al. Carbohydr Res, 17, 25-33 (1971).
83. Potocki de Montalk, G. et al., FEBS Lett, 471, 219-23 (2000).

1.-14. (canceled)

15. A method for preparing the building block corresponding to a disaccharide α-D-glucopyranosyl-(1-4)-N-acetyl-α-D-glucopyranosaminyl of formula (Ia): 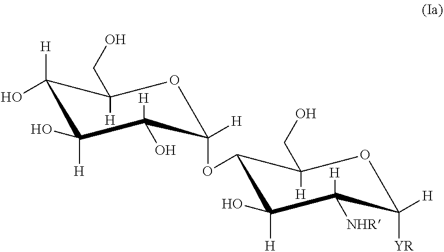
wherein said method comprises the step of reacting:
A) a mutant of a glycoside hydrolase,
wherein said wild type glycoside hydrolase has 450 to 850 amino acids and comprises eleven motifs defined by the following consensus motifs:
(1) the amino acid sequence LGVNYLHLMPL (SEQ ID NO: 1), which is located in the β-strand 2 of said wild type glycoside hydrolase;
(2) the amino acid sequence DGGYAV (SEQ ID NO: 2), which is located in the loop 2 of the (β/α)₈-barrel of said wild type glycoside hydrolase;
(3) the amino acid sequence DFVFNH (SEQ ID NO: 3) which is located in the β-strand 3 of said wild type glycoside hydrolase;
(4) the amino acid sequence LREIFPDTAPGNF (SEQ ID NO: 4), which is located in the domain B of said wild type glycoside hydrolase;
(5) the amino acid sequence FNSYQWDLN (SEQ ID NO: 5), which is located in the C-terminal part of the domain B of said wild type glycoside hydrolase;
(6) the amino acid sequence ILRLDAVAFLWK (SEQ ID NO: 6), which is located in the β-strand 4 of said wild type glycoside hydrolase;
(7) the amino acid sequence EAIV (SEQ ID NO: 7), which is located in the β-strand 5 of said wild type glycoside hydrolase;
(8) the amino acid sequence YVRCHDDI (SEQ ID NO: 8), which is located in the β-strand 7 of said wild type glycoside hydrolase;
(9) the amino acid sequence RISGTLASLAG (SEQ ID NO: 9), which is located in the domain B′ of said wild type glycoside hydrolase;
(10) the amino acid sequence GIPLIYLGDE (SEQ ID NO: 10), which is located in the β-strand 8 of said wild type glycoside hydrolase;
(11) the amino acid sequence RWVHRP (SEQ ID NO: 11), which is located in the loop 8 of the (β/α)₈-barrel,
and the sequence formed by said eleven motifs from said wild type glycoside hydrolase joined end-to-end from motif (1) to motif (11) has at least 65% sequence identity or at least 80% sequence similarity with the amino acid sequence SEQ ID NO: 12;
wherein said mutant has one or two mutation(s) consisting of, when said mutant has only one mutation:
the substitution of the amino acid residue at position 4 in said motif (4) with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V) with the provisio that said wild type glycoside hydrolase does not contain a valine at this position, tryptophan (W) and tyrosine (Y), or
the substitution of the amino acid residue at position 5 in said motif (4) with any amino acid selected from the group consisting of leucine (L), methionine (M) and valine (V), or
the substitution of the amino acid residue at position 8 in said motif (6) with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the amino acid residue at position 9 in said motif (6) with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), or
the substitution of the amino acid residue at position 4 in said motif (7) with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the amino acid residue at position 7 in said motif (8) with any amino acid selected from the group consisting of alanine (A) and valine (V), or
the substitution of the amino acid residue at position 1 in said motif (9) with any amino acid selected from the group consisting of alanine (A), cysteine (C), phenylalanine (F), glycine (G) with the provisio that said wild type glycoside hydrolase does not contain a glycine at this position, lysine (K), asparagine (N), glutamine (Q), serine (S) with the provisio that said wild type glycoside hydrolase does not contain a serine at this position, threonine (T) and tryptophan (W), or
when said mutant has two mutations:
the substitution of the amino acid residue at position 4 in said motif (4) with an alanine (A) and the substitution of the amino acid residue at position 9 in said motif (6) with a histidine (H), or
the substitution of the amino acid residue at position 4 in said motif (4) with a cysteine (C) and the substitution of the amino acid residue at position 5 in said motif (4) with a leucine (L), or
the substitution of the amino acid residue at position 4 in said motif (4) with a lysine (K) and the substitution of the amino acid residue at position 9 in said motif (6) with any amino acid selected from the group consisting of leucine (L) and tryptophan (W), or
the substitution of the amino acid residues at positions 4 and 5 in said motif (4) respectively with a leucine (L), or
the substitution of the amino acid residues at positions 4 and 5 in said motif (4) respectively with a methionine (M), or
the substitution of the amino acid residue at position 4 in said motif (4) with a proline (P) and the substitution of the amino acid residue at position 9 in said motif (6) with a cysteine (C), or
the substitution of the amino acid residue at position 4 in said motif (4) with a threonine (T) and the substitution of the amino acid residue at position 9 in said motif (6) with any amino acid selected from the group consisting of histidine (H) and lysine (K), or
the substitution of the amino acid residue at position 4 in said motif (4) with a valine (V) and the substitution of the amino acid residue at position 5 in said motif (4) with any amino acid selected from the group consisting of leucine (L) and methionine (M), or
the substitution of the amino acid residue at position 4 in said motif (4) with a valine (V) and the substitution of the amino acid residue at position 9 in said motif (6) with any amino acid selected from the group consisting of histidine (H), lysine (K), arginine (R), and valine (V), or
the substitution of the amino acid residue at position 8 in said motif (6) with a histidine (H) and the substitution of the amino acid residue at position 9 in said motif (6) with a serine (S), or
the substitution of the amino acid residue at position 8 in said motif (6) with a proline (P) and the substitution of the amino acid residue at position 9 in said motif (6) with any amino acid selected from the group consisting of cysteine (C), isoleucine (I) and leucine (L), or
the substitution of the amino acid residue at position 8 in said motif (6) with a threonine (T) and the substitution of the amino acid residue at position 9 in said motif (6) with a histidine (H);
B) with the acceptor of formula (IIa): 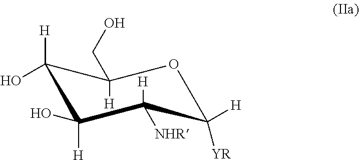
wherein Y is selected from —O— and —S— and R is selected from the group consisting of: C₁-C₆ alkyl, C₁-C₆ alkenyl, aryl, allyl, —CO-alkyl (C₁-C₆), —CO-alkenyl (C₁-C₆), —CO-aryl,
R′ designates a group selected from: acetyl, trichloroacetyl, trifluoroacetyl,
and
with a donor of formula (IIIa): 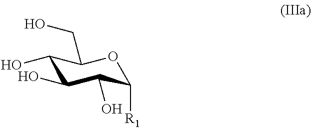
wherein R₁ represents a group selected from:

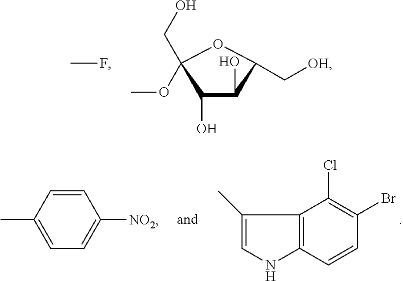

16. A method according to claim 15, wherein said wild type glycoside hydrolase is an amylosucrase (EC 2.4.1.4) or sucrose hydrolase (EC 3.2.1.-).

17. A method according to claim 16, wherein said wild type glycoside hydrolase is an amylosucrase from Neisseria polysaccharea, and is preferably selected from the group consisting of 1G5A (SEQ ID NO: 13), 1ZS2, 1MVY, 1MW0, 1S46, 1JGI, 1MW2, 1MW3, 1MW1 and 1JG9 proteins.

18. A method according to claim 15, for the preparation of the disaccharide α-D-glucopyranosyl-(1→44)-2-N-acetyl-2-deoxy-α-D-glucopyranoside of formula (I): 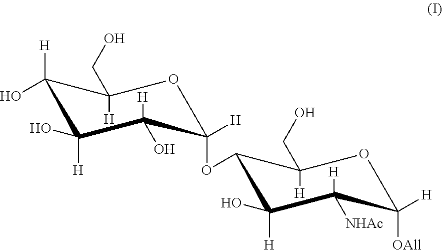
wherein said method comprises the step of reacting a mutant of a glycoside hydrolase as disclosed in claim 15, with the acceptor of formula (II): 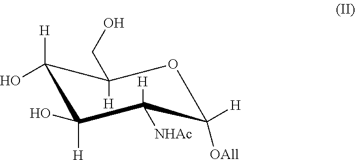
and with a donor sucrose of formula (III):

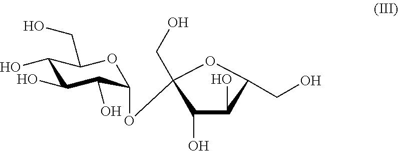

19. A method for the preparation of the building block corresponding to the disaccharide of formula (XX_{3B}) in which R₂ represents a group selected from H, Bn, Ac and AcBn and R₃ represents a group selected from H and Ac. 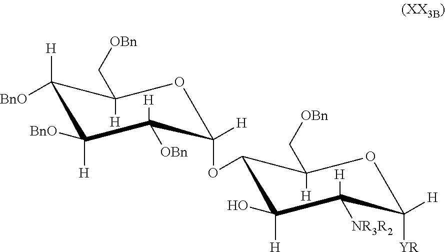
comprising at least one step according to claim 15.

20. A method according to claim 19, for the preparation of the disaccharide of formula (XX_{3A})

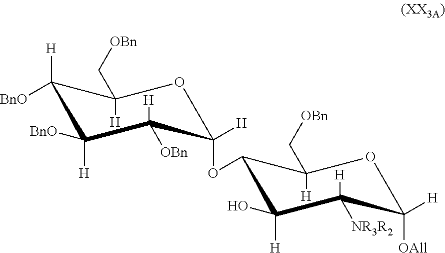

21. A mutant of a wild type glycoside hydrolase, wherein said wild type glycoside hydrolase has 450 to 850 amino acids and comprises eleven motifs defined by the following consensus motifs:
(1) the amino acid sequence LGVNYLHLMPL (SEQ ID NO: 1), which is located in the β-strand 2 of said wild type glycoside hydrolase;
(2) the amino acid sequence DGGYAV (SEQ ID NO: 2), which is located in the loop 2 of the (β/α)₈-barrel of said wild type glycoside hydrolase;
(3) the amino acid sequence DFVFNH (SEQ ID NO: 3) which is located in the β-strand 3 of said wild type glycoside hydrolase;
(4) the amino acid sequence LREIFPDTAPGNF (SEQ ID NO: 4), which is located in the domain B of said wild type glycoside hydrolase;
(5) the amino acid sequence FNSYQWDLN (SEQ ID NO: 5), which is located in the C-terminal part of the domain B of said wild type glycoside hydrolase;
(6) the amino acid sequence ILRLDAVAFLWK (SEQ ID NO: 6), which is located in the β-strand 4 of said wild type glycoside hydrolase;
(7) the amino acid sequence EAIV (SEQ ID NO: 7), which is located in the β-strand 5 of said wild type glycoside hydrolase;
(8) the amino acid sequence YVRCHDDI (SEQ ID NO: 8), which is located in the β-strand 7 of said wild type glycoside hydrolase;
(9) the amino acid sequence RISGTLASLAG (SEQ ID NO: 9), which is located in the domain B′ of said wild type glycoside hydrolase;
(10) the amino acid sequence GIPLIYLGDE (SEQ ID NO: 10), which is located in the β-strand 8 of said wild type glycoside hydrolase;
(11) the amino acid sequence RWVHRP (SEQ ID NO: 11), which is located in the loop 8 of the (β/α)₈-barrel,
and the sequence formed by said eleven motifs from said wild type glycoside hydrolase joined end-to-end from motif (1) to motif (11) has at least 65% sequence identity or at least 80% sequence similarity with the amino acid sequence SEQ ID NO: 12;
wherein said mutant has one or two mutation(s) consisting of, when said mutant has only one mutation:
the substitution of the amino acid residue at position 4 in said motif (4) with any amino acid selected from the group consisting of alanine (A), cysteine (C), glutamic acid (E), glycine (G), histidine (H), leucine (L), methionine (M), asparagine (N), proline (P), glutamine (Q), serine (S), threonine (T), valine (V), tryptophan (W) and tyrosine (Y), or
the substitution of the amino acid residue at position 5 in said motif (4) with any amino acid selected from the group consisting of leucine (L), methionine (M) and valine (V), or
the substitution of the amino acid residue at position 8 in said motif (6) with any amino acid selected from the group consisting of glutamic acid (E), phenylalanine (F), glycine (G), lysine (K), leucine (L), methionine (M), proline (P), glutamine (Q), arginine (R) and valine (V), or
the substitution of the amino acid residue at position 9 in said motif (6) with any amino acid selected from the group consisting of alanine (A), cysteine (C), aspartic acid (D), glutamic acid (E), glycine (G), histidine (H), isoleucine (I), lysine (K), leucine (L), methionine (M), proline (P), glutamine (Q), arginine (R), serine (S), threonine (T), valine (V) and tryptophan (W), or
the substitution of the amino acid residue at position 4 in said motif (7) with any amino acid selected from the group consisting of alanine (A), cysteine (C), aspartic acid (D), glycine (G), histidine (H), isoleucine (I), leucine (L), methionine (M), asparagine (N), serine (S), threonine (T) and tyrosine (Y), or
the substitution of the amino acid residue at position 7 in said motif (8) with a valine (V), or
the substitution of the amino acid residue at position 1 in said motif 9 with any amino acid selected from the group consisting of alanine (A), cysteine (C), phenylalanine (F), glycine (G), lysine (K), asparagine (N), glutamine (Q), serine (S), threonine (T) and tryptophan (W), or
when said mutant has two mutations:
the substitution of the amino acid residue at position 4 in said motif (4) with an alanine (A) and the substitution of the amino acid residue at position 9 in said motif (6) with a histidine (H), or
the substitution of the amino acid residue at position 4 in said motif (4) with a cysteine (C) and the substitution of the amino acid residue at position 5 in said motif (4) with a leucine (L), or
the substitution of the amino acid residue at position 4 in said motif (4) with a lysine (K) and the substitution of the amino acid residue at position 9 in said motif (6) with any amino acid selected from the group consisting of leucine (L) and tryptophan (W), or
the substitution of the amino acid residues at positions 4 and 5 in said motif (4) respectively with a leucine (L), or
the substitution of the amino acid residues at positions 4 and 5 in said motif (4) respectively with a methionine (M), or
the substitution of the amino acid residue at position 4 in said motif (4) with a proline (P) and the substitution of the amino acid residue at position 9 in said motif (6) with a cysteine (C), or
the substitution of the amino acid residue at position 4 in said motif (4) with a threonine (T) and the substitution of the amino acid residue at position 9 in said motif (6) with any amino acid selected from the group consisting of histidine (H) and lysine (K), or
the substitution of the amino acid residue at position 4 in said motif (4) with a valine (V) and the substitution of the amino acid residue at position 5 in said motif (4) with any amino acid selected from the group consisting of leucine (L) and methionine (M), or
the substitution of the amino acid residue at position 4 in said motif (4) with a valine (V) and the substitution of the amino acid residue at position 9 in said motif (6) with any amino acid selected from the group consisting of histidine (H), lysine (K), arginine (R), and valine (V), or
the substitution of the amino acid residue at position 8 in said motif (6) with a histidine (H) and the substitution of the amino acid residue at position 9 in said motif (6) with a serine (S), or
the substitution of the amino acid residue at position 8 in said motif (6) with a proline (P) and the substitution of the amino acid residue at position 9 in said motif (6) with any amino acid selected from the group consisting of cysteine (C), isoleucine (I) and leucine (L), or
the substitution of the amino acid residue at position 8 in said motif (6) with a threonine (T) and the substitution of the amino acid residue at position 9 in said motif (6) with a histidine (H).

22. A mutant according to claim 21, wherein the amino acid residue at position 9 in said motif (6) is substituted with any amino acid selected from the group consisting of cysteine (C), aspartic acid (D), isoleucine (I), lysine (K) and glutamine (Q), and more preferably with any amino acid selected from the group consisting of aspartic acid (D) and lysine (K).

23. A mutant according to claim 21, wherein said wild type glycoside hydrolase is an amylosucrase (EC 2.4.1.4) or sucrose hydrolase (EC 3.2.1.-).

24. A mutant according to claim 23, wherein said wild type glycoside hydrolase is an amylosucrase from Neisseria polysaccharea, and is preferably selected from the group consisting of 1G5A, 1ZS2, 1MVY, 1MW0, 1S46, 1JGI, 1MW2, 1MW3, 1MW1 and 1JG9 proteins.

25. A polynucleotide encoding a mutant of a glycoside hydrolase of claim 21.

26. A recombinant vector comprising a polynucleotide of claim 25.

27. A method for determining whether a wild type protein is a wild type glycoside hydrolase, said method comprising the steps of:
a) determining the amino acid sequence of said protein,
b) identifying in the amino acid sequence of said protein, preferably from the N- to C-terminus, eleven motifs defined by the following consensus motifs:
(SEQ ID NO: 1)

| | |
|---|---|
| (1) | the amino acid sequence LGVNYLHLMPL; |

(SEQ ID NO: 2)

| | |
|---|---|
| (2) | the amino acid sequence DGGYAV; |

(SEQ ID NO: 3)

| | |
|---|---|
| (3) | the amino acid sequence DFVFNH; |

(SEQ ID NO: 4)

| | |
|---|---|
| (4) | the amino acid sequence LREIFPDTAPGNF; |

(SEQ ID NO: 5)

| | |
|---|---|
| (5) | the amino acid sequence FNSYQWDLN; |

(SEQ ID NO: 6)

| | |
|---|---|
| (6) | the amino acid sequence ILRLDAVAFLWK; |

(SEQ ID NO: 7)

| | |
|---|---|
| (7) | the amino acid sequence EAIV; |

(SEQ ID NO: 8)

| | |
|---|---|
| (8) | the amino acid sequence YVRCHDDI; |

(SEQ ID NO: 9)

| | |
|---|---|
| (9) | the amino acid sequence RISGTLASLAG |

(SEQ ID NO: 10)

| | |
|---|---|
| (10) | the amino acid sequence GIPLIYLGDE; |

(SEQ ID NO: 11)

| | |
|---|---|
| (11) | the amino acid sequence RWVHRP; |

determining the sequence identity percent or sequence similarity percent between the sequence formed by said eleven motifs joined end-to-end from motif (1) to motif (11) with the amino acid sequence SEQ ID NO: 12, and if the sequence identity percent is at least 65%, or if the sequence similarity percent is at least 80%, then the wild type protein is a wild type glycoside hydrolase.

28. A molecule selected from the group consisting of:
Allyl α-D-glucopyranosyl-(1→4)-α-D-glucopyranosyl-(1→4)-2-acetamido-2-deoxy-α-D-glucopyranoside (XX₂),
Allyl α-D-glucopyranosyl-(1→4)-2-acetamido-2-deoxy-α-D-glucopyranoside (XX₃),
Allyl 2,3,4,6-tetra-O-acetyl-α-D-glucopyranosyl-(1→4)-2-acetamido-3,6-di-O-acetyl-2-deoxy-α-D-glucopyranoside (XX₄),
Allyl α-D-glucopyranosyl-(1→4)-2-amino-2-deoxy-α-D-glucopyranoside (XX₅),
Allyl α-D-glucopyranosyl-(1→4)-2-amino-2-N,3-O-carbonyl-2-deoxy-α-D-glucopyranoside (XX₆),
Allyl 2,4,6-tri-O-benzyl-α-D-glucopyranosyl-(1→4)-6-O-benzyl-2-benzylamino-2-N,3-O-carbonyl-2-deoxy-α-D-glucopyranoside (XX₇),
Allyl 2,3,4,6-tetra-O-benzyl-α-D-glucopyranosyl-(1-*4)-6-O-benzyl-2-benzylamino-2-N,3-O-carbonyl-2-deoxy-α-D-glucopyranoside (XX₈),
Allyl 2,3,4,6-tetra-O-benzyl-α-D-glucopyranosyl-(1→4)-6-O-benzyl-2-amino-2-N,3-O-carbonyl-2-deoxy-α-D-glucopyranoside (XX_{8A}),
Allyl 2,3,4,6-tetra-O-benzyl-α-D-glucopyranosyl-(1→4)-6-O-benzyl-2-benzylamino-2-deoxy-α-D-glucopyranoside (XX₉),
Allyl 2,3,4,6-tetra-O-benzyl-α-D-glucopyranosyl-(1→4)-6-O-benzyl-2-benzylacetamido-2-deoxy-α-D-glucopyranoside (XX₁₀),
Allyl 2-O-benzoyl-4-O-benzyl-3-O-chloroacetyl-α-L-rhamnopyranosyl-(1→3)-[2,3,4,6-tetra-O-benzyl-α-D-glucopyranosyl-(1→4)]-6-O-benzyl-2-benzylacetamido-2-deoxy-α-D-glucopyranoside (XX₁₁),
Allyl 2-O-acetyl-3,4-di-O-benzyl-α-L-rhamnopyranosyl-(1→3)[2,3,4,6-tetra-O-benzyl-α-D-glucopyranosyl-(1→4)]-6-O-benzyl-2-benzylacetamido-2-deoxy-α-D-glucopyranoside (XX₁₂),
Allyl α-D-glucopyranosyl-(1→4)-2-deoxy-2-trichloroacetamido-α-D-glucopyranoside (XX₁₃),
Allyl 2,3,4,6-tetra-O-acetyl-α-D-glucopyranosyl-(1→4)-3,6-di-O-acetyl-2-deoxy-2-trichloroacetamido-α-D-glucopyranoside (XX₁₄),
2,3,4,6-tetra-O-acetyl-α-D-glucopyranosyl-(1→4)-3,6-di-O-acetyl-2-deoxy-2-trichloroacetamido-α-D-glucopyranose (XX₁₅),
2,3,4,6-Tetra-O-acetyl-α-D-glucopyranosyl-(1→4)-3,6-di-O-acetyl-2-deoxy-2-trichloroacetamido-α-D-glucopyranosyl trichloroacetimidate (XX₁₆),
Allyl 2,3,4,6-tetra-O-acetyl-α-D-glucopyranosyl-(1→4)-3,6-di-O-acetyl-2-deoxy-2-trichloroacetamido-α-D-glucopyranosyl-(1→2)-3,4-di-O-benzyl-α-L-rhamnopyranoside (XX₁₇),
Allyl 2,3,4,6-tetra-O-benzyl-α-D-glucopyranosyl-(1→4)-2-acetamido-3-O-acetyl-6-O-benzyl-2-deoxy-α-D-glucopyranoside (XX₂₄), and
Allyl 2,3,4,6-tetra-O-benzyl-α-D-glucopyranosyl-(1→4)-2-acetamido-6-O-benzyl-2-deoxy-α-D-glucopyranoside (XX₂₅).
